(19) United States
(12) Patent Application Publication
(10) Pub. No.: US 2022/0401390 A1
(43) Pub. Date: Dec. 22, 2022

(54) TREATMENTS OF DIABETIC MACULAR EDEMA AND IMPAIRED VISUAL ACUITY

(71) Applicant: KALVISTA PHARMACEUTICALS LIMITED, Salisbury (GB)

(72) Inventors: Edward Paul FEENER, North Reading, MA (US); Michael David SMITH, Salt Lake City, UT (US); Christopher Martyn YEA, Salisbury (GB)

(21) Appl. No.: 17/783,703

(22) PCT Filed: Dec. 9, 2020
(86) PCT No.: PCT/GB2020/053153

§ 371 Date: Jun. 9, 2022

Related U.S. Application Data
(60) Provisional application No. 62/945,560, filed on Dec. 9, 2019.

(30) Foreign Application Priority Data
Dec. 20, 2019 (GB) 1918994.3

(51) Int. Cl. A61K 31/166; A61P 3/08; A61K 9/00

(52) U.S. Cl. CPC A61K 31/166; A61P 3/08; A61K 9/0019

(57) ABSTRACT

The present invention relates to treatments of diabetic macular edema (DME) and impaired visual acuity, comprising intravitreally administering the compound of formula (A) (or a pharmaceutically acceptable salt and/or solvate thereof): Formula (A).

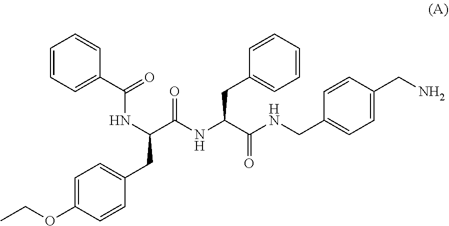 [SMILES: CCOc1ccc(C[C@@H](NC(=O)c2ccccc2)C(=O)N[C@@H](Cc2ccccc2)C(=O)NCc2ccc(CN)cc2)cc1]

The present invention relates to treatments of diabetic macular edema (DME) and impaired visual acuity.

### BACKGROUND OF THE INVENTION

Visual acuity refers, in its broadest sense, to clarity of vision. Visual acuity is dependent on optical and neural factors, i.e. the sharpness of the retinal focus within the eye, the health and functioning of the retina, and the sensitivity of the interpretative faculty of the brain. Numerous medical conditions cause impaired visual acuity. Examples of these conditions include diabetic macular edema (DME), diabetic retinopathy, retinal vascular permeability associated with diabetic retinopathy, retinal vascular occlusion, diabetes, macular degeneration and neuropathy.

Diabetic macular edema (DME) is a common complication of diabetes mellitus. It leads to vision loss, if untreated, and becomes increasingly prevalent with progressing diabetes. In 2015, over 30 million Americans were estimated to be affected by diabetes and almost 10 million by diabetic retinopathy; 1.5 million were estimated to have vision-threatening diabetic retinopathy and 908,000 of these would have DME (Lee, Wong et al. 2015). DME is the leading cause of moderate vision loss among working age adults in most developed countries (Diabetes, Complications Trial/Epidemiology of Diabetes et al. 2009).

The clinical signs of diabetic retinopathy begin with retinal hemorrhages and micro-aneurysms, usually associated with areas of retinal pericyte loss and loss of the endothelial cell barrier function. The resulting leakage can lead to macular edema, consisting of an accumulation of fluid and lipoproteins in the retina. Visual acuity declines dramatically when the central macula is affected.

The eyes of patients with diabetic macular edema are associated with highly elevated levels of plasma kallikrein, as well as VEGF, however the roles plasma kallikrein and VEGF are well understood to be independent of one another (Kita et al., Diabetes 2015).

The plasma kallikrein-kinin system is a system of blood proteins that plays a role in inflammation, blood pressure control, coagulation and pain. The plasma kallikrein-kinin system is abnormally abundant in patients with advanced diabetic macular edema. It has recently been published that plasma kallikrein contributes to retinal vascular dysfunctions in diabetic rats (A. Clermont et al. “Plasma kallikrein mediates retinal vascular dysfunction and induces retinal thickening in diabetic rats” Diabetes, 2011, 60, p 1590-98). Furthermore, administration of the plasma kallikrein inhibitor ASP-440 ameliorated both retinal vascular permeability and retinal blood flow abnormalities in diabetic rats. Therefore, a plasma kallikrein inhibitor should have utility as a treatment to reduce retinal vascular permeability associated with diabetic retinopathy and diabetic macular edema. Other complications of diabetes such as cerebral haemorrhage, nephropathy, cardiomyopathy and neuropathy, all of which have associations with plasma kallikrein may also be considered as targets for a plasma kallikrein inhibitor.

Synthetic and small molecule plasma kallikrein inhibitors have been described previously, for example by Garrett et al. (“Peptide aldehyde . . . ” J. Peptide Res. 52, p 62-71 (1998)), T. Griesbacher et al. (“Involvement of tissue kallikrein but not plasma kallikrein in the development of symptoms mediated by endogenous kinins in acute pancreatitis in rats” British Journal of Pharmacology 137, p 692-700 (2002)), Evans (“Selective dipeptide inhibitors of kallikrein” WO03/076458), Szelke et al. (“Kininogenase inhibitors” WO92/04371), D. M. Evans et al. (Immunolpharmacology, 32, p 115-116 (1996)), Szelke et al. (“Kininogen inhibitors” WO95/07921), Antonsson et al. (“New peptides derivatives” WO94/29335), J. Corte et al. (“Six membered heterocycles useful as serine protease inhibitors” WO2005/123680), J. Stürzbecher et al. (Brazilian J. Med. Biol. Res 27, p 1929-34 (1994)), Kettner et al. (U.S. Pat. No. 5,187,157), N. Teno et al. (Chem. Pharm. Bull. 41, p 1079-1090 (1993)), W. B. Young et al. (“Small molecule inhibitors of plasma kallikrein” Bioorg. Med. Chem. Letts. 16, p 2034-2036 (2006)), Okada et al. (“Development of potent and selective plasmin and plasma kallikrein inhibitors and studies on the structure-activity relationship” Chem. Pharm. Bull. 48, p 1964-72 (2000)), Steinmetzer et al. (“Trypsin-like serine protease inhibitors and their preparation and use” WO08/049595), Zhang et al. (“Discovery of highly potent small molecule kallikrein inhibitors” Medicinal Chemistry 2, p 545-553 (2006)), Sinha et al. (“Inhibitors of plasma kallikrein” WO08/016883), Shigenaga et al. (“Plasma Kallikrein Inhibitors” WO2011/118672), and Kolte et al. (“Biochemical characterization of a novel high-affinity and specific kallikrein inhibitor”, British Journal of Pharmacology (2011), 162(7), 1639-1649). Also, Steinmetzer et al. (“Serine protease inhibitors” WO2012/004678) describes cyclized peptide analogs which are inhibitors of human plasmin and plasma kallikrein.

To date, only two selective plasma kallikrein inhibitors have been approved for medical use: Ecallantide and Lanadelumab. Ecallantide is formulated as a solution for injection. It is a large protein plasma kallikrein inhibitor that presents a risk of anaphylactic reactions. Lanadelumab is a human monoclonal antibody, used in the prevention of angioedema in patients with hereditary angioedema, and is also formulated as a solution for injection. Neither Ecallantide nor Lanadelumab have been investigated or approved for the treatment of diabetic macular edema. Other plasma kallikrein inhibitors known in the art are generally small molecules, some of which include highly polar and ionisable functional groups, such as guanidines or amidines. Recently, plasma kallikrein inhibitors that do not feature guanidine or amidine functionalities have been reported. For example Brandl et al. (“N-((6-amino-pyridin-3-yl)methyl)-heteroaryl-carboxamides as inhibitors of plasma kallikrein” WO2012/017020), Evans et al. (“Benzylamine derivatives as inhibitors of plasma kallikrein” WO2013/005045), Allan et al. (“Benzylamine derivatives” WO2014/108679), and Davie et al. (“Heterocyclic derivates” WO2014/188211).

Intravitreal injection of plasma kallikrein inhibitors is known (for example, see Evans et al. WO2013/005045) and allows the plasma kallikrein inhibitor to be delivered directly to the ocular tissues. However, small molecules dosed as solutions and administered by intravitreal injection are typically cleared from the vitreous within hours (for example, see “Review: Practical Issues in Intravitreal Drug Delivery”, Journal of Ocular Pharmacology and Therapeutics, Volume 17, Number 4, 2001, p 393-401, David Maurice and “Prediction of Vitreal Half-Life Based on Drug Physiochemical Properties: Quantitative Structure-Pharmacokinetic Relationships (QSPKR)”, Pharmaceutical Research, Volume 26, Number 5, 2009, p 1236-1260, Chandrasekar Durairaj et al.).

Intravitreal injection is an invasive procedure, and therefore reduced clearance and an extended duration of action are desirable to increase the period required between injections. Cook et al. (“Pharmaceutical compositions” WO2014/108685) discloses compositions containing suspended plasma kallikrein inhibitors with relatively long dissolution times, thus providing a relatively long period of action. However, a problem with pharmaceutical compositions containing suspended actives is that additional manufacturing steps are required, such as reducing the particle size of the active ingredient and controlling the particle size distribution of the active ingredient. There is also a risk of non-homogeneity of the suspension in the formulation.

Plasma kallikrein inhibitors with a longer duration of action are disclosed in WO2019/030540. These inhibitors do not have the disadvantages associated with a suspension of active ingredient. The pharmaceutical compositions comprising the disclosed plasma kallikrein inhibitors are suitable for injection into the eye, and have a long duration of action in the ocular tissues, particularly the retina.

Therapies for DME directed against the vascular endothelial growth factor (anti-VEGF therapies) have made a significant difference in the treatment of DME (Campochiaro, Aiello et al. 2016). The treatments currently in use, e.g. aflibercept (Eylea®), bevacizumab, ranibizumab and pegaptanib, were shown in clinical trials to be more effective than laser therapy after one year. However, a significant proportion (up to 50%) of patients with DME does not achieve vision gain under anti-VEGF therapy (Nguyen, Brown et al. 2012).

A treatment for DME can be measured for its efficacy by its impact on visual acuity. Visual acuity is the symptom of the disease observed by the patients, thus any change in the patient's visual acuity, or slowing of the progression of the deterioration of said visual acuity is an improvement in the treatment of the disease.

Therefore, there remains a need for alternative treatments for impaired visual acuity. There also remains a need for alternative treatments of diabetic macular edema. There also remains a need for treatments of diabetic macular edema and visual acuity that slow the progression of the condition, or prevent deterioration of the patient's condition.

### SUMMARY OF THE INVENTION

It is an object of the present invention to provide a treatment of impaired visual acuity that slows the progression of the condition or prevents deterioration of the patient's condition.

It is a further object of the present invention to provide a treatment of diabetic macular edema that slows the progression of the condition or prevents deterioration of the patient's condition.

Surprisingly, it has been found that intravitreal administration of a pharmaceutical composition which is a solution, preferably an aqueous solution, which comprises the compound of Formula A (or a pharmaceutically acceptable salt and/or solvate thereof) is effective and well tolerated in patients with DME or impaired visual acuity. In this regard, the present invention provides an alternative option where a DME patient or a patient with impaired visual acuity is no longer clinically recommended to use a previous therapy, and the patient is not receiving the previous therapy, in particular an anti-VEGF therapy. For instance, the previous treatment with a different therapy, in particular anti-VEGF therapy, may no longer be clinically recommended because the previous therapy was not tolerated for whatever reason (e.g. because adverse effects have been experienced). Alternatively, or additionally, the previous treatment with a different therapy, in particular an anti-VEGF therapy, may no longer be clinically recommended because the previous treatment did not result in at least a slowing of the progression of the DME or impaired visual acuity. Surprisingly, this treatment has been found to be particularly effective where the patients are in the early stages of DME or impaired visual acuity. It has also been found that by administering an appropriate dose, it may be possible to reduce the frequency of intravitreal injections of the pharmaceutical composition of Formula A (or a pharmaceutically acceptable salt and/or solvate thereof) described herein required in order to prevent progression of the disease. Intravitreal injections are uncomfortable for the patient, and require administration by a medical professional (i.e. cannot be self-administered), therefore a reduction in the frequency of these intravitreal injections is advantageous.

### DESCRIPTION OF THE INVENTION

The invention is defined by the appended claims.

In a first aspect, the present invention relates to a method for treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

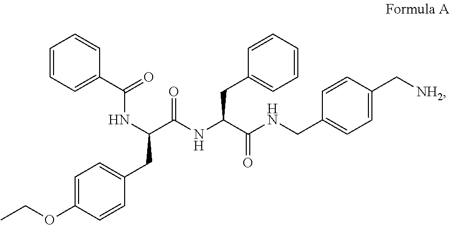 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.

The present invention also relates to a compound of Formula A (or a pharmaceutically acceptable salt and/or solvate thereof) for use in treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

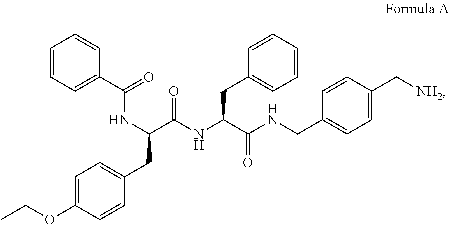 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.

The present invention also relates to a use of the compound of Formula A (or a pharmaceutically acceptable salt and/or solvate thereof) in the manufacture of a medicament for the treatment of diabetic macular edema (DME), comprising: intravitreally administering a pharmaceutical composition, wherein the pharmaceutical composition is a solution comprising the compound of Formula A (or a pharmaceutically acceptable salt and/or solvate thereof), to a patient in need thereof,

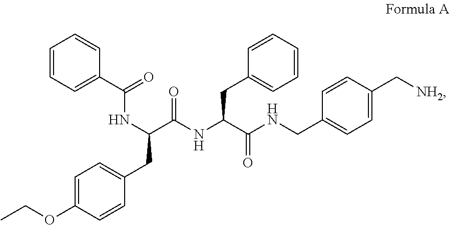 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.

The pharmaceutical composition is preferably an aqueous solution comprising the compound of Formula A (or a pharmaceutically acceptable salt and/or solvate thereof).

The intravitreal administration preferably comprises intravitreal injection. The intravitreal administration is preferably into at least one of the patient's eyes. The intravitreal administration is may also be into both of the patient's eyes.

The formulation of the pharmaceutical composition is as defined below. Preferably, the pharmaceutical composition is an aqueous solution containing the compound of formula A (or a pharmaceutically acceptable salt and/or solvate thereof), histidine and trehalose dihydrate.

The pH of the composition is preferably from about 2 to about 10, more preferably from about 5 to about 7.5, even more preferably from about 5.3 to about 6, yet more preferably from about 5.4 to about 5.8 and most preferably about 5.5.

The concentration of the compound of formula A (or a pharmaceutically acceptable salt and/or solvate thereof) when administered is based on the concentration of the free base of the compound of formula A in solution.

The concentration of the compound of formula A (or a pharmaceutically acceptable salt and/or solvate thereof) when administered may be between about 10 μg/mL and about 300 μg/mL. Preferably, the concentration of the compound of formula A (or a pharmaceutically acceptable salt and/or solvate thereof) when administered may be between about 10 μg/mL and about 200 μg/mL. More preferably, the concentration of the compound of formula A (or a pharmaceutically acceptable salt and/or solvate thereof) when administered may be between about 30 μg/mL and about 100 μg/mL. Yet more preferably, the concentration of the compound of formula A (or a pharmaceutically acceptable salt and/or solvate thereof) when administered may be between about 60 μg/mL and about 100 μg/mL. The concentration of the compound of formula A (or a pharmaceutically acceptable salt and/or solvate thereof) when administered may be about 30 μg/mL. The concentration of the compound of formula A (or a pharmaceutically acceptable salt and/or solvate thereof) when administered may be about 60 μg/m L. The concentration of the compound of formula A (or a pharmaceutically acceptable salt and/or solvate thereof) when administered may be about 100 μg/mL.

Up to about 100 μL of the solution is administered per intravitreal administration. Preferably between about 10 μL to about 100 μL of the solution is administered per intravitreal administration. More preferably between about 25 μL to about 100 μL of the solution is administered per intravitreal administration. More preferably about 50 μL to about 100 μL of the solution is administered per intravitreal administration.

Preferably between about 50 μL to about 60 μL of the solution is administered per intravitreal administration. Preferably between about 60 μL to about 70 μL of the solution is administered per intravitreal administration. Preferably between about 70 μL to about 80 μL of the solution is administered per intravitreal administration. Preferably between about 80 μL to about 90 μL of the solution is administered per intravitreal administration. Preferably between about 90 μL to about 100 μL of the solution is administered per intravitreal administration. Preferably, about 50 μL of the solution is administered per intravitreal administration. Preferably, about 60 μL of the solution is administered per intravitreal administration. Preferably, about 70 μL of the solution is administered per intravitreal administration. Preferably, about 80 μL of the solution is administered per intravitreal administration. Preferably, about 90 μL of the solution is administered per intravitreal administration. Preferably, about 100 μL of the solution is administered per intravitreal administration.

The patient may be in the early stage of DME. The early stage of DME may be defined by the patient having a baseline visual acuity score (BCVA), prior to administration of the compound of Formula A, of between 56 and 73 letters in at least one eye, measured using the standard Early Treatment Diabetic Retinopathy Study (ETDRS) chart.

The treatment can comprise administering the compound of Formula A (or a pharmaceutically acceptable salt and/or solvate thereof) in combination with an anti-VEGF treatment. For example, the anti-VEGF treatment received in combination can be selected from aflibercept (Eylea®), bevacizumab, ranibizumab, and pegaptanib. The anti-VEGF treatment received in combination can be administered in the same pharmaceutical composition as the compound of Formula A (or a pharmaceutically acceptable salt and/or solvate thereof). Alternatively, the anti-VEGF treatment received in combination can be administered in a different pharmaceutical composition to the compound of Formula A (or a pharmaceutically acceptable salt and/or solvate thereof). The different pharmaceutical compositions can be administered separately, sequentially or simultaneously.

Preferably, the treatment with the pharmaceutical composition which is a solution comprising the compound of Formula A (or a pharmaceutically acceptable salt and/or solvate thereof) is a monotherapy for DME.

Preferably, the patient does not receive anti-VEGF treatment concurrent to administration of the compound of Formula A (or a pharmaceutically acceptable salt and/or solvate thereof).

Preferably, the previous anti-VEGF treatment was for treating impaired visual acuity or DME. For example, the anti-VEGF treatment can be aflibercept (Eylea®), bevacizumab, ranibizumab, and pegaptanib.

Preferably, the previous anti-VEGF treatment commenced no more than 36 months before the treatment with the compound of Formula A (or a pharmaceutically acceptable salt and/or solvate thereof). Preferably, the patient received anti-VEGF treatment no less than 8 weeks before commencing treatment with the compound of formula A (or a pharmaceutically acceptable salt and/or solvate thereof).

The treatment with the pharmaceutical composition which is a solution comprising the compound of Formula A (or a pharmaceutically acceptable salt and/or solvate thereof) can be administered over any time period, and can be administered indefinitely or for life. Preferably, the treatment is administered over a time period of at least about 12 weeks.

The treatment with the pharmaceutical composition which is a solution comprising the compound of Formula A (or a pharmaceutically acceptable salt and/or solvate thereof) can be administered at a first dosing frequency over a first time period, followed by a second dosing frequency over a second time period, wherein the second dosing frequency is lower than the first dosing frequency. The first time period is preferably greater than about 8 weeks, and more preferably greater than about 12 weeks. The first dosing frequency can be between about once every three weeks and about once every five weeks. The second time period can be greater than about 8 weeks, greater than about 12 weeks, greater than about 16 weeks, between about 8 weeks and about 16 weeks, between about 8 weeks and about 12 weeks or about 12 weeks. The second dosing frequency is preferably lower than about once every six weeks.

Alternatively, the treatment with the pharmaceutical composition which is a solution comprising the compound of Formula A (or a pharmaceutically acceptable salt and/or solvate thereof) can be administered at a regular frequency of between about once every 4 weeks and about once every 12 weeks. Preferably, the treatment is administered about once every 4 weeks.

Preferably, treatment with the pharmaceutical composition which is a solution comprising the compound of Formula A (or a pharmaceutically acceptable salt and/or solvate thereof) slows the progression of DME.

In a second aspect, the present invention relates to a method for treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

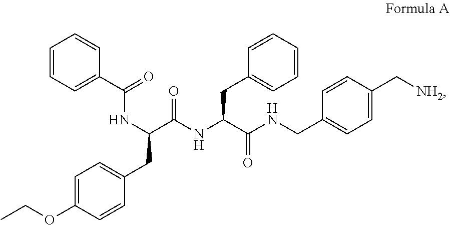 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.

The present invention also relates to a compound of Formula A (or a pharmaceutically acceptable salt and/or solvate thereof) for use in treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

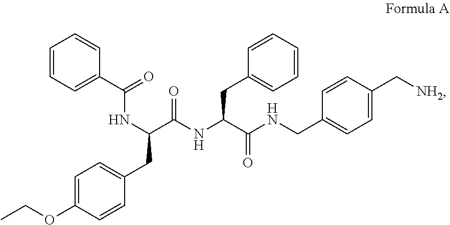 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.

The present invention also relates to a use of the compound of Formula A (or a pharmaceutically acceptable salt and/or solvate thereof) in the manufacture of a medicament for the treatment of impaired visual acuity, comprising: intravitreally administering a pharmaceutical composition, wherein the pharmaceutical composition is a solution comprising the compound of Formula A (or a pharmaceutically acceptable salt and/or solvate thereof), to a patient in need thereof,

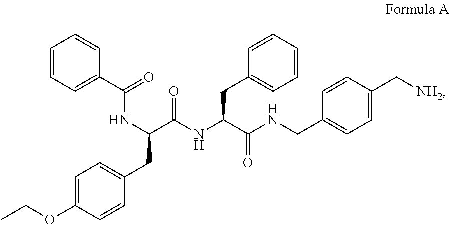 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.

The pharmaceutical composition is preferably an aqueous solution comprising the compound of Formula A (or a pharmaceutically acceptable salt and/or solvate thereof).

The intravitreal administration preferably comprises intravitreal injection. The intravitreal administration is preferably into at least one of the patient's eyes. The intravitreal administration is may also be into both of the patient's eyes.

The formulation of the pharmaceutical composition is as defined below. Preferably, the pharmaceutical composition is an aqueous solution containing the compound of formula A (or a pharmaceutically acceptable salt and/or solvate thereof), histidine and trehalose dihydrate.

The pH of the composition is preferably from about 2 to about 10, more preferably from about 5 to about 7.5, even more preferably from about 5.3 to about 6, yet more preferably from about 5.4 to about 5.8 and most preferably about 5.5.

The concentration of the compound of formula A (or a pharmaceutically acceptable salt and/or solvate thereof) when administered is based on the concentration of the free base of the compound of formula A in solution.

The concentration of the compound of formula A (or a pharmaceutically acceptable salt and/or solvate thereof) when administered may be between about 10 μg/mL and about 300 μg/mL. Preferably, the concentration of the compound of formula A (or a pharmaceutically acceptable salt and/or solvate thereof) when administered may be between about 10 μg/mL and about 200 μg/mL. More preferably, the concentration of the compound of formula A (or a pharmaceutically acceptable salt and/or solvate thereof) when administered may be between about 30 μg/mL and about 100 μg/mL. Yet more preferably, the concentration of the compound of formula A (or a pharmaceutically acceptable salt and/or solvate thereof) when administered may be between about 60 μg/mL and about 100 μg/mL. The concentration of the compound of formula A (or a pharmaceutically acceptable salt and/or solvate thereof) when administered may be about 30 μg/mL. The concentration of the compound of formula A (or a pharmaceutically acceptable salt and/or solvate thereof) when administered may be about 60 μg/m L. The concentration of the compound of formula A (or a pharmaceutically acceptable salt and/or solvate thereof) when administered may be about 100 μg/mL.

Up to about 100 μL of the solution is administered per intravitreal administration. Preferably between about 10 μL to about 100 μL of the solution is administered per intravitreal administration. More preferably between about 25 μL to about 100 μL of the solution is administered per intravitreal administration. More preferably about 50 μL to about 100 μL of the solution is administered per intravitreal administration.

Preferably between about 50 μL to about 60 μL of the solution is administered per intravitreal administration. Preferably between about 60 μL to about 70 μL of the solution is administered per intravitreal administration. Preferably between about 70 μL to about 80 μL of the solution is administered per intravitreal administration. Preferably between about 80 μL to about 90 μL of the solution is administered per intravitreal administration. Preferably between about 90 μL to about 100 μL of the solution is administered per intravitreal administration. Preferably, about 50 μL of the solution is administered per intravitreal administration. Preferably, about 60 μL of the solution is administered per intravitreal administration. Preferably, about 70 μL of the solution is administered per intravitreal administration. Preferably, about 80 μL of the solution is administered per intravitreal administration. Preferably, about 90 μL of the solution is administered per intravitreal administration. Preferably, about 100 μL of the solution is administered per intravitreal administration

The patient may be in the early stage of impaired visual acuity. The early stage of impaired visual acuity may be defined by the patient having a baseline visual acuity score (BCVA), prior to administration of the compound of Formula A, of between 56 and 73 letters in at least one eye, measured using the standard Early Treatment Diabetic Retinopathy Study (ETDRS) chart.

The treatment can comprise administering the compound of Formula A (or a pharmaceutically acceptable salt and/or solvate thereof) in combination with an anti-VEGF treatment. For example, the anti-VEGF treatment received in combination can be selected from aflibercept (Eylea®), bevacizumab, ranibizumab, and pegaptanib. The anti-VEGF treatment received in combination can be administered in the same pharmaceutical composition as the compound of Formula A (or a pharmaceutically acceptable salt and/or solvate thereof). Alternatively, the anti-VEGF treatment received in combination can be administered in a different pharmaceutical composition to the compound of Formula A (or a pharmaceutically acceptable salt and/or solvate thereof). The different pharmaceutical compositions can be administered separately, sequentially or simultaneously.

Preferably, the treatment with the pharmaceutical composition which is a solution comprising the compound of Formula A (or a pharmaceutically acceptable salt and/or solvate thereof) is a monotherapy for impaired visual acuity.

Preferably, the patient does not receive anti-VEGF treatment concurrent to administration of the compound of Formula A (or a pharmaceutically acceptable salt and/or solvate thereof).

Preferably, the previous anti-VEGF treatment was for treating impaired visual acuity or DME. For example, the anti-VEGF treatment can be aflibercept (Eylea®), bevacizumab, ranibizumab, and pegaptanib.

Preferably, the previous anti-VEGF treatment commenced no more than 36 months before the treatment with the compound of Formula A (or a pharmaceutically acceptable salt and/or solvate thereof). Preferably, the patient received anti-VEGF treatment no less than 8 weeks before commencing treatment with the compound of formula A (or a pharmaceutically acceptable salt and/or solvate thereof).

The treatment with the pharmaceutical composition which is a solution comprising the compound of Formula A (or a pharmaceutically acceptable salt and/or solvate thereof) can be administered over any time period, and can be administered indefinitely or for life. Preferably, the treatment is administered over a time period of at least about 12 weeks.

The treatment with the pharmaceutical composition which is a solution comprising the compound of Formula A (or a pharmaceutically acceptable salt and/or solvate thereof) can be administered at a first dosing frequency over a first time period, followed by a second dosing frequency over a second time period, wherein the second dosing frequency is lower than the first dosing frequency. The first time period is preferably greater than about 8 weeks, and more preferably greater than about 12 weeks. The first dosing frequency can be between about once every three weeks and about once every five weeks. The second time period can be greater than about 8 weeks, greater than about 12 weeks, greater than about 16 weeks, between about 8 weeks and about 16 weeks, between about 8 weeks and about 12 weeks or about 12 weeks. The second dosing frequency is preferably lower than about once every six weeks.

Alternatively, the treatment with the pharmaceutical composition which is a solution comprising the compound of Formula A (or a pharmaceutically acceptable salt and/or solvate thereof) can be administered at a regular frequency of between about once every 4 weeks and about once every 12 weeks. Preferably, the treatment is administered about once every 4 weeks.

Preferably, treatment with the pharmaceutical composition which is a solution comprising the compound of Formula A (or a pharmaceutically acceptable salt and/or solvate thereof) slows the progression of impaired visual acuity.

In a third aspect, the present invention relates to a method for treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

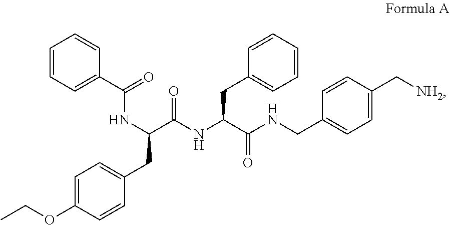 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of DME.

The present invention also relates to a compound of Formula A (or a pharmaceutically acceptable salt and/or solvate thereof) for use in treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

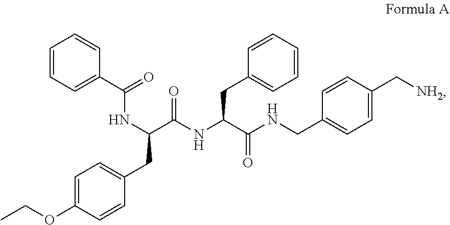 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of DME.

The present invention also relates to a use of the compound of Formula A (or a pharmaceutically acceptable salt and/or solvate thereof) in the manufacture of a medicament for the treatment of diabetic macular edema (DME), comprising: intravitreally administering a pharmaceutical composition, wherein the pharmaceutical composition is a solution comprising the compound of Formula A (or a pharmaceutically acceptable salt and/or solvate thereof), to a patient in need thereof,

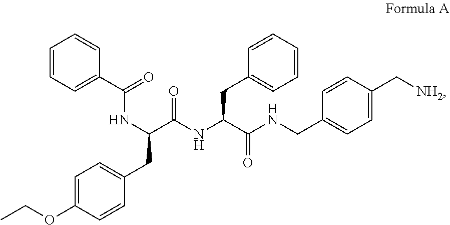 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of DME.

The pharmaceutical composition is preferably an aqueous solution comprising the compound of Formula A (or a pharmaceutically acceptable salt and/or solvate thereof).

The intravitreal administration preferably comprises intravitreal injection. The intravitreal administration is preferably into at least one of the patient's eyes. The intravitreal administration may also be into both of the patient's eyes.

The formulation of the pharmaceutical composition is as defined below. Preferably, the pharmaceutical composition is an aqueous solution containing the compound of formula A (or a pharmaceutically acceptable salt and/or solvate thereof), histidine and trehalose dihydrate.

The pH of the composition is preferably from about 2 to about 10, more preferably from about 5 to about 7.5, even more preferably from about 5.3 to about 6, yet more preferably from about 5.4 to about 5.8 and most preferably about 5.5.

The concentration of the compound of formula A (or a pharmaceutically acceptable salt and/or solvate thereof) when administered is based on the concentration of the free base of the compound of formula A in solution.

The concentration of the compound of formula A (or a pharmaceutically acceptable salt and/or solvate thereof) when administered may be between about 10 μg/mL and about 300 μg/mL. Preferably, the concentration of the compound of formula A (or a pharmaceutically acceptable salt and/or solvate thereof) when administered may be between about 10 μg/mL and about 200 μg/mL. More preferably, the concentration of the compound of formula A (or a pharmaceutically acceptable salt and/or solvate thereof) when administered may be between about 30 μg/mL and about 100 μg/mL. Yet more preferably, the concentration of the compound of formula A (or a pharmaceutically acceptable salt and/or solvate thereof) when administered may be between about 60 μg/mL and about 100 μg/mL. The concentration of the compound of formula A (or a pharmaceutically acceptable salt and/or solvate thereof) when administered may be about 30 μg/m L. The concentration of the compound of formula A (or a pharmaceutically acceptable salt and/or solvate thereof) when administered may be about 60 μg/m L. The concentration of the compound of formula A (or a pharmaceutically acceptable salt and/or solvate thereof) when administered may be about 100 μg/mL.

Up to about 100 μL of the solution is administered per intravitreal administration. Preferably between about 10 μL to about 100 μL of the solution is administered per intravitreal administration. More preferably between about 25 μL to about 100 μL of the solution is administered per intravitreal administration. More preferably about 50 μL to about 100 μL of the solution is administered per intravitreal administration.

Preferably between about 50 μL to about 60 μL of the solution is administered per intravitreal administration. Preferably between about 60 μL to about 70 μL of the solution is administered per intravitreal administration. Preferably between about 70 μL to about 80 μL of the solution is administered per intravitreal administration. Preferably between about 80 μL to about 90 μL of the solution is administered per intravitreal administration. Preferably between about 90 μL to about 100 μL of the solution is administered per intravitreal administration. Preferably, about 50 μL of the solution is administered per intravitreal administration. Preferably, about 60 μL of the solution is administered per intravitreal administration. Preferably, about 70 μL of the solution is administered per intravitreal administration. Preferably, about 80 μL of the solution is administered per intravitreal administration. Preferably, about 90 μL of the solution is administered per intravitreal administration. Preferably, about 100 μL of the solution is administered per intravitreal administration.

The patient is in the early stages of DME. The early stage of DME may be defined by the patient having a baseline visual acuity score (BCVA), prior to administration of the compound of Formula A, of between 56 and 73 letters in at least one eye, measured using the standard Early Treatment Diabetic Retinopathy Study (ETDRS) chart.

The treatment can comprise administering the compound of Formula A (or a pharmaceutically acceptable salt and/or solvate thereof) in combination with an anti-VEGF treatment. For example, the anti-VEGF treatment received in combination can be selected from aflibercept (Eylea®), bevacizumab, ranibizumab, and pegaptanib. The anti-VEGF treatment received in combination can be administered in the same pharmaceutical composition as the compound of Formula A (or a pharmaceutically acceptable salt and/or solvate thereof). Alternatively, the anti-VEGF treatment received in combination can be administered in a different pharmaceutical composition to the compound of Formula A (or a pharmaceutically acceptable salt and/or solvate thereof). The different pharmaceutical compositions can be administered separately, sequentially or simultaneously.

Preferably, the treatment with the pharmaceutical composition which is a solution comprising the compound of Formula A (or a pharmaceutically acceptable salt and/or solvate thereof) is a monotherapy for DME.

Preferably, the patient does not receive anti-VEGF treatment concurrent to administration of the compound of Formula A (or a pharmaceutically acceptable salt and/or solvate thereof).

The patient may previously have had anti-VEGF treatment.

Preferably, the previous anti-VEGF treatment was for treating impaired visual acuity or DME. For example, the anti-VEGF treatment can be aflibercept (Eylea®), bevacizumab, ranibizumab, and pegaptanib.

Preferably, the previous anti-VEGF treatment commenced no more than 36 months before the treatment with the compound of Formula A (or a pharmaceutically acceptable salt and/or solvate thereof). Preferably, the patient received anti-VEGF treatment no less than 8 weeks before commencing treatment with the compound of formula A (or a pharmaceutically acceptable salt and/or solvate thereof).

The treatment with the pharmaceutical composition which is a solution comprising the compound of Formula A (or a pharmaceutically acceptable salt and/or solvate thereof) can be administered over any time period, and can be administered indefinitely or for life. Preferably, the treatment is administered over a time period of at least about 12 weeks.

The treatment with the pharmaceutical composition which is a solution comprising the compound of Formula A (or a pharmaceutically acceptable salt and/or solvate thereof) can be administered at a first dosing frequency over a first time period, followed by a second dosing frequency over a second time period, wherein the second dosing frequency is lower than the first dosing frequency. The first time period is preferably greater than about 8 weeks, and more preferably greater than about 12 weeks. The first dosing frequency can be between about once every three weeks and about once every five weeks. The second time period can be greater than about 8 weeks, greater than about 12 weeks, greater than about 16 weeks, between about 8 weeks and about 16 weeks, between about 8 weeks and about 12 weeks or about 12 weeks. The second dosing frequency is preferably lower than about once every six weeks.

Alternatively, the treatment with the pharmaceutical composition which is a solution comprising the compound of Formula A (or a pharmaceutically acceptable salt and/or solvate thereof) can be administered at a regular frequency of between about once every 4 weeks and about once every 12 weeks. Preferably, the treatment is administered about once every 4 weeks.

Preferably, treatment with the pharmaceutical composition which is a solution comprising the compound of Formula A (or a pharmaceutically acceptable salt and/or solvate thereof) slows the progression of DME.

In a fourth aspect, the present invention relates to a method for treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

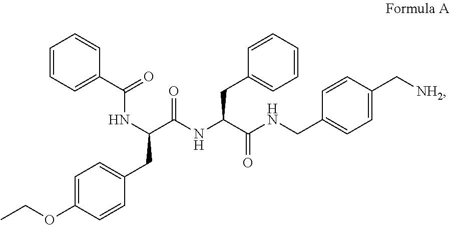 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of impaired visual acuity.

The present invention also relates to a compound of Formula A (or a pharmaceutically acceptable salt and/or solvate thereof) for use in treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

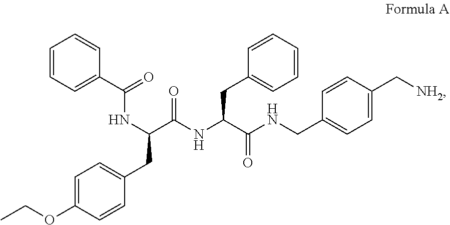 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of impaired visual acuity.

The present invention also relates to a use of the compound of Formula A (or a pharmaceutically acceptable salt and/or solvate thereof) in the manufacture of a medicament for the treatment of impaired visual acuity, comprising: intravitreally administering a pharmaceutical composition, wherein the pharmaceutical composition is a solution comprising the compound of Formula A (or a pharmaceutically acceptable salt and/or solvate thereof), to a patient in need thereof,

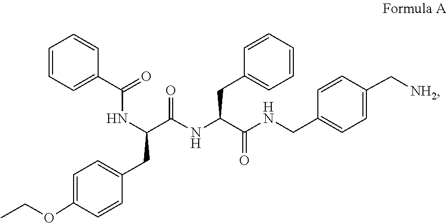 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of impaired visual acuity.

The pharmaceutical composition is preferably an aqueous solution comprising the compound of Formula A (or a pharmaceutically acceptable salt and/or solvate thereof).

The intravitreal administration preferably comprises intravitreal injection. The intravitreal administration is preferably into at least one of the patient's eyes. The intravitreal administration is may also be into both of the patient's eyes.

The formulation of the pharmaceutical composition is as defined below. Preferably, the pharmaceutical composition is an aqueous solution containing the compound of formula A (or a pharmaceutically acceptable salt and/or solvate thereof), histidine and trehalose dihydrate.

The pH of the composition is preferably from about 2 to about 10, more preferably from about 5 to about 7.5, even more preferably from about 5.3 to about 6, yet more preferably from about 5.4 to about 5.8 and most preferably about 5.5.

The concentration of the compound of formula A (or a pharmaceutically acceptable salt and/or solvate thereof) when administered is based on the concentration of the free base of the compound of formula A in solution.

The concentration of the compound of formula A (or a pharmaceutically acceptable salt and/or solvate thereof) when administered may be between about 10 μg/mL and about 300 μg/mL. Preferably, the concentration of the compound of formula A (or a pharmaceutically acceptable salt and/or solvate thereof) when administered may be between about 10 μg/mL and about 200 μg/mL. More preferably, the concentration of the compound of formula A (or a pharmaceutically acceptable salt and/or solvate thereof) when administered may be between about 30 μg/mL and about 100 μg/mL. Yet more preferably, the concentration of the compound of formula A (or a pharmaceutically acceptable salt and/or solvate thereof) when administered may be between about 60 μg/mL and about 100 μg/mL. The concentration of the compound of formula A (or a pharmaceutically acceptable salt and/or solvate thereof) when administered may be about 30 μg/mL. The concentration of the compound of formula A (or a pharmaceutically acceptable salt and/or solvate thereof) when administered may be about 60 μg/mL. The concentration of the compound of formula A (or a pharmaceutically acceptable salt and/or solvate thereof) when administered may be about 100 μg/mL.

Up to about 100 μL of the solution is administered per intravitreal administration. Preferably between about 10 μL to about 100 μL of the solution is administered per intravitreal administration. More preferably between about 25 μL to about 100 μL of the solution is administered per intravitreal administration. More preferably about 50 μL to about 100 μL of the solution is administered per intravitreal administration.

Preferably between about 50 μL to about 60 μL of the solution is administered per intravitreal administration. Preferably between about 60 μL to about 70 μL of the solution is administered per intravitreal administration. Preferably between about 70 μL to about 80 μL of the solution is administered per intravitreal administration. Preferably between about 80 μL to about 90 μL of the solution is administered per intravitreal administration. Preferably between about 90 μL to about 100 μL of the solution is administered per intravitreal administration. Preferably, about 50 μL of the solution is administered per intravitreal administration. Preferably, about 60 μL of the solution is administered per intravitreal administration. Preferably, about 70 μL of the solution is administered per intravitreal administration. Preferably, about 80 μL of the solution is administered per intravitreal administration. Preferably, about 90 μL of the solution is administered per intravitreal administration. Preferably, about 100 μL of the solution is administered per intravitreal administration.

The patient is in the early stages of impaired visual acuity. The early stage of impaired visual acuity may be defined by the patient having a baseline visual acuity score (BCVA), prior to administration of the compound of Formula A, of between 56 and 73 letters in at least one eye, measured using the standard Early Treatment Diabetic Retinopathy Study (ETDRS) chart.

The treatment can comprise administering the compound of Formula A (or a pharmaceutically acceptable salt and/or solvate thereof) in combination with an anti-VEGF treatment. For example, the anti-VEGF treatment received in combination can be aflibercept (Eylea®), bevacizumab, ranibizumab, and pegaptanib. The anti-VEGF treatment received in combination can be selected from administered in the same pharmaceutical composition as the compound of Formula A (or a pharmaceutically acceptable salt and/or solvate thereof). Alternatively, the anti-VEGF treatment received in combination can be administered in a different pharmaceutical composition to the compound of Formula A (or a pharmaceutically acceptable salt and/or solvate thereof). The different pharmaceutical compositions can be administered separately, sequentially or simultaneously.

Preferably, the treatment with the pharmaceutical composition which is a solution comprising the compound of Formula A (or a pharmaceutically acceptable salt and/or solvate thereof) is a monotherapy for impaired visual acuity.

Preferably, the patient does not receive anti-VEGF treatment concurrent to administration of the compound of Formula A (or a pharmaceutically acceptable salt and/or solvate thereof).

The patient may have had previous anti-VEGF treatment.

Preferably, the previous anti-VEGF treatment was for treating impaired visual acuity or DME. For example, the anti-VEGF treatment can be aflibercept (Eylea®), bevacizumab, ranibizumab, and pegaptanib.

Preferably, the previous anti-VEGF treatment commenced no more than 36 months before the treatment with the compound of Formula A (or a pharmaceutically acceptable salt and/or solvate thereof). Preferably, the patient received anti-VEGF treatment no less than 8 weeks before commencing treatment with the compound of formula A (or a pharmaceutically acceptable salt and/or solvate thereof).

The treatment with the pharmaceutical composition which is a solution comprising the compound of Formula A (or a pharmaceutically acceptable salt and/or solvate thereof) can be administered over any time period, and can be administered indefinitely or for life. Preferably, the treatment is administered over a time period of at least about 12 weeks.

The treatment with the pharmaceutical composition which is a solution comprising the compound of Formula A (or a pharmaceutically acceptable salt and/or solvate thereof) can be administered at a first dosing frequency over a first time period, followed by a second dosing frequency over a second time period, wherein the second dosing frequency is lower than the first dosing frequency. The first time period is preferably greater than about 8 weeks, and more preferably greater than about 12 weeks. The first dosing frequency can be between about once every three weeks and about once every five weeks. The second time period can be greater than about 8 weeks, greater than about 12 weeks, greater than about 16 weeks, between about 8 weeks and about 16 weeks, between about 8 weeks and about 12 weeks or about 12 weeks. The second dosing frequency is preferably lower than about once every six weeks.

Alternatively, the treatment with the pharmaceutical composition which is a solution comprising the compound of Formula A (or a pharmaceutically acceptable salt and/or solvate thereof) can be administered at a regular frequency of between about once every 4 weeks and about once every 12 weeks. Preferably, the treatment is administered about once every 4 weeks.

Preferably, treatment with the pharmaceutical composition which is a solution comprising the compound of Formula A (or a pharmaceutically acceptable salt and/or solvate thereof) slows the progression of impaired visual acuity.

In a fifth aspect, the present invention relates to a method for treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

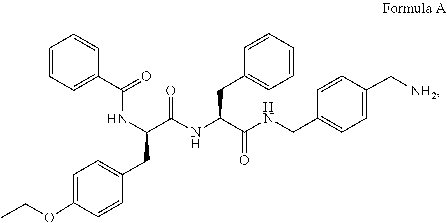 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

The present invention also relates to a compound of Formula A (or a pharmaceutically acceptable salt and/or solvate thereof) for use in treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

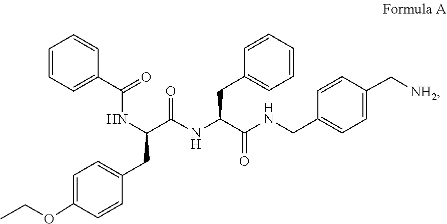 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

The present invention also relates to a use of the compound of Formula A (or a pharmaceutically acceptable salt and/or solvate thereof) in the manufacture of a medicament for the treatment of diabetic macular edema (DME), comprising: intravitreally administering a pharmaceutical composition, wherein the pharmaceutical composition is a solution comprising the compound of Formula A (or a pharmaceutically acceptable salt and/or solvate thereof), to a patient in need thereof,

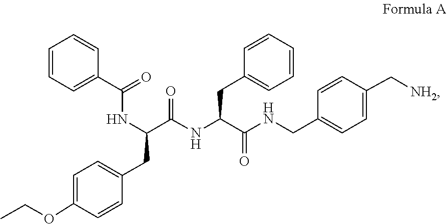 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

Preferably, the concentration of the compound of formula A (or a pharmaceutically acceptable salt and/or solvate thereof) when administered in the first time period is between about 60 μg/mL and about 300 μg/mL based on the concentration of the free base of the compound of formula A in solution. Preferably, the concentration of the compound of formula A (or a pharmaceutically acceptable salt and/or solvate thereof) when administered in the first time period is between about 60 μg/mL and about 200 μg/mL based on the concentration of the free base of the compound of formula A in solution. Preferably, the concentration of the compound of formula A (or a pharmaceutically acceptable salt and/or solvate thereof) when administered in the first time period is between about 60 μg/mL and about 100 μg/mL based on the concentration of the free base of the compound of formula A in solution. In particular, the concentration of the compound of formula A (or a pharmaceutically acceptable salt and/or solvate thereof) when administered in the first time period may be about 60 μg/mL. In particular, the concentration of the compound of formula A (or a pharmaceutically acceptable salt and/or solvate thereof) when administered in the first time period may be about 100 μg/mL.

The concentration of the compound of formula A (or a pharmaceutically acceptable salt and/or solvate thereof) when administered in the second time period may be between about 10 μg/mL and about 300 μg/mL. Preferably, the concentration of the compound of formula A (or a pharmaceutically acceptable salt and/or solvate thereof) when administered in the second time period may be between about 10 μg/mL and about 200 μg/mL. More preferably, the concentration of the compound of formula A (or a pharmaceutically acceptable salt and/or solvate thereof) when administered in the second time period may be between about 30 μg/mL and about 100 μg/mL. Yet more preferably, the concentration of the compound of formula A (or a pharmaceutically acceptable salt and/or solvate thereof) when administered may be between about 60 μg/mL and about 100 μg/mL. The concentration of the compound of formula A (or a pharmaceutically acceptable salt and/or solvate thereof) when administered in the second time period may be about 30 μg/mL. The concentration of the compound of formula A (or a pharmaceutically acceptable salt and/or solvate thereof) when administered in the second time period may be about 60 μg/mL. The concentration of the compound of formula A (or a pharmaceutically acceptable salt and/or solvate thereof) when administered in the second time period may be about 100 μg/mL.

The first time period can be greater than about 24 weeks. Preferably, the first time period is greater than about 20 weeks. Preferably, the first time period is greater than about 16 weeks. Preferably, the first time period is greater than about 12 weeks. Most preferably, the first time period is greater than about 8 weeks.

The first time period can be between about 8 weeks and about 20 weeks. The first time period can be between about 8 weeks and about 16 weeks. The first time period can be between about 10 weeks and about 14 weeks. The first time period can be between about 10 weeks and about 12 weeks. The first time period can be about 12 weeks.

Preferably, the second time period can be greater than about 8 weeks, greater than about 12 weeks, greater than about 16 weeks, between about 8 weeks and about 16 weeks, between about 8 weeks and about 12 weeks or about 12 weeks.

Preferably, the first dosing frequency is between about once every two weeks, and about once every 6 weeks. More preferably, the first dosing frequency is between about once every three weeks and about once every five weeks. Most preferably, the first dosing frequency is about once every 4 weeks.

The second dosing frequency is lower than the first dosing frequency. For example, the second dosing frequency may be about once every six weeks, about once every eight weeks, about once every ten weeks or about once every twelve weeks. Preferably, the second dosing frequency is lower than about once every six weeks. More preferably, the second dosing frequency is lower than about once every eight weeks. Most preferably, the second dosing frequency is lower than about once every 12 weeks.

The pharmaceutical composition is preferably an aqueous solution comprising the compound of Formula A (or a pharmaceutically acceptable salt and/or solvate thereof).

The intravitreal administration preferably comprises intravitreal injection. The intravitreal administration is preferably into at least one of the patient's eyes. The intravitreal administration is may also be into both of the patient's eyes.

The formulation of the pharmaceutical composition is as defined below. Preferably, the pharmaceutical composition is an aqueous solution containing the compound of formula A (or a pharmaceutically acceptable salt and/or solvate thereof), histidine and trehalose dihydrate.

The pH of the composition is preferably from about 2 to about 10, more preferably from about 5 to about 7.5, even more preferably from about 5.3 to about 6, yet more preferably from about 5.4 to about 5.8 and most preferably about 5.5.

The concentration of the compound of formula A (or a pharmaceutically acceptable salt and/or solvate thereof) when administered is based on the concentration of the free base of the compound of formula A in solution.

Up to about 100 μL of the solution is administered per intravitreal administration. Preferably between about 10 μL to about 100 μL of the solution is administered per intravitreal administration. More preferably between about 25 μL to about 100 μL of the solution is administered per intravitreal administration. More preferably about 50 μL to about 100 μL of the solution is administered per intravitreal administration.

Preferably between about 50 μL to about 60 μL of the solution is administered per intravitreal administration. Preferably between about 60 μL to about 70 μL of the solution is administered per intravitreal administration. Preferably between about 70 μL to about 80 μL of the solution is administered per intravitreal administration. Preferably between about 80 μL to about 90 μL of the solution is administered per intravitreal administration. Preferably between about 90 μL to about 100 μL of the solution is administered per intravitreal administration. Preferably, about 50 μL of the solution is administered per intravitreal administration. Preferably, about 60 μL of the solution is administered per intravitreal administration. Preferably, about 70 μL of the solution is administered per intravitreal administration. Preferably, about 80 μL of the solution is administered per intravitreal administration. Preferably, about 90 μL of the solution is administered per intravitreal administration. Preferably, about 100 μL of the solution is administered per intravitreal administration.

The patient may be in the early stage of DME. The early stage of DME may be defined by the patient having a baseline visual acuity score (BCVA), prior to administration of the compound of Formula A, of between 56 and 73 letters in at least one eye, measured using the standard Early Treatment Diabetic Retinopathy Study (ETDRS) chart.

The treatment can comprise administering the compound of Formula A (or a pharmaceutically acceptable salt and/or solvate thereof) in combination with an anti-VEGF treatment. For example, the anti-VEGF treatment received in combination can be selected from aflibercept (Eylea®), bevacizumab, ranibizumab, and pegaptanib. The anti-VEGF treatment received in combination can be administered in the same pharmaceutical composition as the compound of Formula A (or a pharmaceutically acceptable salt and/or solvate thereof). Alternatively, the anti-VEGF treatment received in combination can be administered in a different pharmaceutical composition to the compound of Formula A (or a pharmaceutically acceptable salt and/or solvate thereof). The different pharmaceutical compositions can be administered separately, sequentially or simultaneously.

Preferably, the treatment with the pharmaceutical composition which is a solution comprising the compound of Formula A (or a pharmaceutically acceptable salt and/or solvate thereof) is a monotherapy for DME.

Preferably, the patient does not receive anti-VEGF treatment concurrent to administration of the compound of Formula A (or a pharmaceutically acceptable salt and/or solvate thereof).

The patient may have had previous anti-VEGF treatment.

Preferably, the previous anti-VEGF treatment was for treating impaired visual acuity or DME. For example, the anti-VEGF treatment can be aflibercept (Eylea®), bevacizumab, ranibizumab, and pegaptanib.

Preferably, the previous anti-VEGF treatment commenced no more than 36 months before the treatment with the compound of Formula A (or a pharmaceutically acceptable salt and/or solvate thereof). Preferably, the patient received anti-VEGF treatment no less than 8 weeks before commencing treatment with the compound of formula A (or a pharmaceutically acceptable salt and/or solvate thereof).

Preferably, treatment with the pharmaceutical composition which is a solution comprising the compound of Formula A (or a pharmaceutically acceptable salt and/or solvate thereof) slows the progression of DME.

In a sixth aspect, the present invention relates to a method for treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

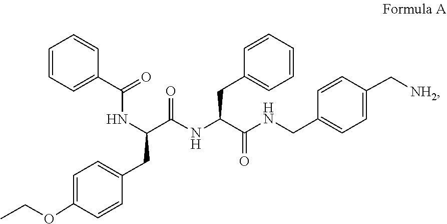 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

The present invention also relates to a compound of Formula A (or a pharmaceutically acceptable salt and/or solvate thereof) for use in treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

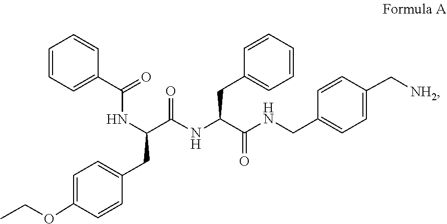 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

The present invention also relates to a use of the compound of Formula A (or a pharmaceutically acceptable salt and/or solvate thereof) in the manufacture of a medicament for the treatment of impaired visual acuity, comprising: intravitreally administering a pharmaceutical composition, wherein the pharmaceutical composition is a solution comprising the compound of Formula A (or a pharmaceutically acceptable salt and/or solvate thereof), to a patient in need thereof,

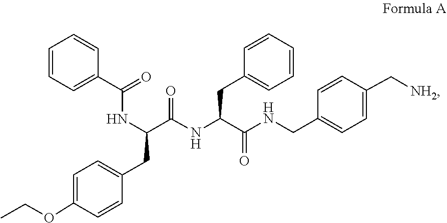 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

Preferably, the concentration of the compound of formula A (or a pharmaceutically acceptable salt and/or solvate thereof) when administered in the first time period is between about 60 μg/mL and about 300 μg/mL based on the concentration of the free base of the compound of formula A in solution. Preferably, the concentration of the compound of formula A (or a pharmaceutically acceptable salt and/or solvate thereof) when administered in the first time period is between about 60 μg/mL and about 200 μg/mL based on the concentration of the free base of the compound of formula A in solution. Preferably, the concentration of the compound of formula A (or a pharmaceutically acceptable salt and/or solvate thereof) when administered in the first time period is between about 60 μg/mL and about 100 μg/mL based on the concentration of the free base of the compound of formula A in solution. In particular, the concentration of the compound of formula A (or a pharmaceutically acceptable salt and/or solvate thereof) when administered in the first time period may be about 60 μg/mL. In particular, the concentration of the compound of formula A (or a pharmaceutically acceptable salt and/or solvate thereof) when administered in the first time period may be about 100 μg/mL.

The concentration of the compound of formula A (or a pharmaceutically acceptable salt and/or solvate thereof) when administered in the second time period may be between about 10 μg/mL and about 300 μg/mL. Preferably, the concentration of the compound of formula A (or a pharmaceutically acceptable salt and/or solvate thereof) when administered in the second time period may be between about 10 μg/mL and about 200 μg/mL. More preferably, the concentration of the compound of formula A (or a pharmaceutically acceptable salt and/or solvate thereof) when administered in the second time period may be between about 30 μg/mL and about 100 μg/mL. Yet more preferably, the concentration of the compound of formula A (or a pharmaceutically acceptable salt and/or solvate thereof) when administered may be between about 60 μg/mL and about 100 μg/mL. The concentration of the compound of formula A (or a pharmaceutically acceptable salt and/or solvate thereof) when administered in the second time period may be about 30 μg/mL. The concentration of the compound of formula A (or a pharmaceutically acceptable salt and/or solvate thereof) when administered in the second time period may be about 60 μg/mL. The concentration of the compound of formula A (or a pharmaceutically acceptable salt and/or solvate thereof) when administered in the second time period may be about 100 μg/mL.

The first time period can be greater than about 24 weeks. Preferably, the first time period is greater than about 20 weeks. Preferably, the first time period is greater than about 16 weeks. Preferably, the first time period is greater than about 12 weeks. Most preferably, the first time period is greater than about 8 weeks.

The first time period can be between about 8 weeks and about 20 weeks. The first time period can be between about 8 weeks and about 16 weeks. The first time period can be between about 10 weeks and about 14 weeks. The first time period can be between about 10 weeks and about 12 weeks. The first time period can be about 12 weeks.

Preferably, the second time period is greater than about 8 weeks, greater than about 12 weeks, greater than about 16 weeks, between about 8 weeks and about 16 weeks, between about 8 weeks and about 12 weeks or about 12 weeks.

Preferably, the first dosing frequency is between about once every two weeks, and about once every 6 weeks. More preferably, the first dosing frequency is between about once every three weeks and about once every five weeks. Most preferably, the first dosing frequency is about once every 4 weeks.

The second dosing frequency is lower than the first dosing frequency. For example, the second dosing frequency may be about once every six weeks, about once every eight weeks, about once every ten weeks or about once every twelve weeks. Preferably, the second dosing frequency is lower than about once every six weeks. More preferably, the second dosing frequency is lower than about once every eight weeks. Most preferably, the second dosing frequency is lower than about once every 12 weeks.

The pharmaceutical composition is preferably an aqueous solution comprising the compound of Formula A (or a pharmaceutically acceptable salt and/or solvate thereof).

The intravitreal administration preferably comprises intravitreal injection. The intravitreal administration is preferably into at least one of the patient's eyes. The intravitreal administration is may also be into both of the patient's eyes.

The formulation of the pharmaceutical composition is as defined below. Preferably, the pharmaceutical composition is an aqueous solution containing the compound of formula A (or a pharmaceutically acceptable salt and/or solvate thereof), histidine and trehalose dihydrate.

The pH of the composition is preferably from about 2 to about 10, more preferably from about 5 to about 7.5, even more preferably from about 5.3 to about 6, yet more preferably from about 5.4 to about 5.8 and most preferably about 5.5.

The concentration of the compound of formula A (or a pharmaceutically acceptable salt and/or solvate thereof) when administered is based on the concentration of the free base of the compound of formula A in solution.

Up to about 100 μL of the solution is administered per intravitreal administration. Preferably between about 10 μL to about 100 μL of the solution is administered per intravitreal administration. More preferably between about 25 μL to about 100 μL of the solution is administered per intravitreal administration. More preferably about 50 μL to about 100 μL of the solution is administered per intravitreal administration.

Preferably between about 50 μL to about 60 μL of the solution is administered per intravitreal administration. Preferably between about 60 μL to about 70 μL of the solution is administered per intravitreal administration. Preferably between about 70 μL to about 80 μL of the solution is administered per intravitreal administration. Preferably between about 80 μL to about 90 μL of the solution is administered per intravitreal administration. Preferably between about 90 μL to about 100 μL of the solution is administered per intravitreal administration. Preferably, about 50 μL of the solution is administered per intravitreal administration. Preferably, about 60 μL of the solution is administered per intravitreal administration. Preferably, about 70 μL of the solution is administered per intravitreal administration. Preferably, about 80 μL of the solution is administered per intravitreal administration. Preferably, about 90 μL of the solution is administered per intravitreal administration. Preferably, about 100 μL of the solution is administered per intravitreal administration.

The patient may be in the early stage of impaired visual acuity. The early stage of impaired visual acuity may be defined by the patient having a baseline visual acuity score (BCVA), prior to administration of the compound of Formula A, of between 56 and 73 letters in at least one eye, measured using the standard Early Treatment Diabetic Retinopathy Study (ETDRS) chart.

The treatment can comprise administering the compound of Formula A (or a pharmaceutically acceptable salt and/or solvate thereof) in combination with an anti-VEGF treatment. For example, the anti-VEGF treatment received in combination can be selected from aflibercept (Eylea®), bevacizumab, ranibizumab, and pegaptanib. The anti-VEGF treatment received in combination can be administered in the same pharmaceutical composition as the compound of Formula A (or a pharmaceutically acceptable salt and/or solvate thereof). Alternatively, the anti-VEGF treatment received in combination can be administered in a different pharmaceutical composition to the compound of Formula A (or a pharmaceutically acceptable salt and/or solvate thereof). The different pharmaceutical compositions can be administered separately, sequentially or simultaneously.

Preferably, the treatment with the pharmaceutical composition which is a solution comprising the compound of Formula A (or a pharmaceutically acceptable salt and/or solvate thereof) is a monotherapy for impaired visual acuity.

Preferably, the patient does not receive anti-VEGF treatment concurrent to administration of the compound of Formula A (or a pharmaceutically acceptable salt and/or solvate thereof).

The patient may have had previous anti-VEGF treatment.

Preferably, the previous anti-VEGF treatment was for treating impaired visual acuity or DME. For example, the anti-VEGF treatment can be aflibercept (Eylea®), bevacizumab, ranibizumab, and pegaptanib.

Preferably, the previous anti-VEGF treatment commenced no more than 36 months before the treatment with the compound of Formula A (or a pharmaceutically acceptable salt and/or solvate thereof). Preferably, the patient received anti-VEGF treatment no less than 8 weeks before commencing treatment with the compound of formula A (or a pharmaceutically acceptable salt and/or solvate thereof).

Preferably, treatment with the pharmaceutical composition which is a solution comprising the compound of Formula A (or a pharmaceutically acceptable salt and/or solvate thereof) slows the progression of impaired visual acuity.

### Treatment of Impaired Visual Acuity

The uses and methods of the invention are useful for the treatment of impaired visual acuity. In particular, the uses and methods of the invention are useful for slowing the progression of impaired visual acuity. Impaired visual acuity encompasses any medical condition whose symptoms involve a decrease in visual acuity. For example, said impaired visual acuity may be measured by Best Corrected Visual Acuity (BCVA) with the Early Treatment of Diabetic Retinopathy Study. Examples of conditions with symptoms of impaired visual acuity include diabetic macular edema, diabetic retinopathy, retinal vascular permeability associated with diabetic retinopathy, retinal vascular occlusion, diabetes, macular degeneration and neuropathy.

The uses and methods of the invention are useful as a safe and tolerated treatment for impaired visual acuity.

The uses and methods of the invention are useful for the treatment of impaired visual acuity in patients that have previously had anti-VEGF treatment.

The uses and methods of the invention are useful for the treatment of impaired visual acuity in patients that have previously had anti-VEGF treatment for impaired visual acuity or DME.

The uses and methods of the invention are useful for the treatment of impaired visual acuity in patients that are in the early stages of impaired visual acuity. The early stages of impaired visual acuity may be characterised by having a baseline visual acuity BCVA value of between 56 and 73.

The uses and methods of the invention are useful for the treatment of impaired visual acuity where the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

In this regard, the uses and methods of the invention provide an alternative treatment for patients suffering from impaired visual acuity, in particular where patients have been previously treated with a different therapy (e.g. anti-VEGF) and that different therapy is no longer clinically recommended, and the patient is not receiving the different therapy concurrent to the treatment of the uses and methods of the invention. For instance, the previous treatment with a different therapy (e.g. anti-VEGF) may no longer be clinically recommended because the previous treatment was not tolerated for whatever reason (e.g. because adverse effects have been experienced). Alternatively, or additionally, the previous treatment with a different therapy (e.g. anti-VEGF) may no longer be clinically recommended because the previous treatment did not result in at least a slowing of the progression of the impaired visual acuity.

Particularly, the uses and methods of the invention provide an alternative treatment for patients suffering from impaired visual acuity, in particular where patients have been previously treated with an anti-VEGF therapy and the anti-VEGF therapy is no longer clinically recommended, and the patient is not receiving the different therapy concurrent to the treatment of the uses and methods of the invention. For instance, the previous anti-VEGF therapy may no longer be clinically recommended because the previous treatment was not tolerated for whatever reason (e.g. because adverse effects have been experienced). Alternatively, or additionally, the previous anti-VEGF therapy may no longer be clinically recommended because the previous treatment did not result in at least a slowing of the progression of the impaired visual acuity. Preferably, the previous anti-VEGF therapy was for treating impaired visual acuity and DME.

### Treatment of Diabetic Macular Edema (DME)

The uses and methods of the invention are useful for the treatment of diabetic macular edema. In particular, the uses and methods of the invention are useful for slowing the progression of DME. In some embodiments, the uses and methods are useful for the treatment of microvascular complications of a disease state.

The uses and methods of the invention are useful as a safe and tolerated treatment for DME.

The uses and methods of the invention are useful for the treatment of diabetic macular edema in patients that have previously had anti-VEGF treatment.

The uses and methods of the invention are useful for the treatment of DME in patients that have previously had anti-VEGF treatment for impaired visual acuity or DME.

The uses and methods of the invention are useful for the treatment of diabetic macular edema in patients that are in the early stages of DME. The early stages of DME may be characterised by having a baseline visual acuity BCVA value of between 56 and 73.

The uses and methods of the invention are useful for the treatment of diabetic macular edema where the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

In this regard, the uses and methods of the invention provide an alternative treatment for patients suffering from DME, in particular where patients have been previously treated with a different therapy (e.g. anti-VEGF) and that different therapy is no longer clinically recommended, and the patient is not receiving the different therapy concurrent to the treatment of the uses and methods of the invention. For instance, the previous treatment with a different therapy (e.g. anti-VEGF) may no longer be clinically recommended because the previous treatment was not tolerated for whatever reason (e.g. because adverse effects have been experienced). Alternatively, or additionally, the previous treatment with a different therapy (e.g. anti-VEGF) may no longer be clinically recommended because the previous treatment did not result in at least a slowing of the progression of the DME.

Particularly, the uses and methods of the invention provide an alternative treatment for patients suffering from DME, in particular where patients have been previously treated with an anti-VEGF therapy and the anti-VEGF therapy is no longer clinically recommended, and the patient is not receiving the different therapy concurrent to the treatment of the uses and methods of the invention. For instance, the previous anti-VEGF therapy may no longer be clinically recommended because the previous treatment was not tolerated for whatever reason (e.g. because adverse effects have been experienced). Alternatively, or additionally, the previous anti-VEGF therapy may no longer be clinically recommended because the previous treatment did not result in at least a slowing of the progression of the DME. Preferably, the previous anti-VEGF therapy was for treating impaired visual acuity and DME.

### Administration

The uses and methods of the invention involve intravitreal administration. Accordingly, the compound of Formula A is administered into the eye. The compound of Formula A may be administered into one eye, at least one eye, or into both eyes.

Suitable devices for parenteral administration include needle (including microneedle) injectors, needle-free injectors and infusion techniques.

The uses and methods may involve administering the compound of Formula A in the form of sterile aqueous solutions. The preparation of parenteral formulations under sterile conditions, for example, by lyophilisation and reconstitution, may readily be accomplished using standard pharmaceutical techniques well known to those skilled in the art. For example, a suitable method for sterilising the compositions of the present invention may be terminal sterilisation, or sterile filtration followed by aseptic fill-finish. The terminal sterilisation method, sterile filtration and aseptic processing are described in US Pharmacopeia USP<1211> Sterilization and Sterility Assurance of Compendial Articles and terminal sterilisation is further described in US Pharmacopeia USP<1222> Terminally Sterilized Pharmaceutical Products-Parametric Release. (See United States Pharmacopeia (USP) 37, NF 32).

The compositions may be administered to the patient under the supervision of an attending physician.

In any of the treatments of the invention described herein, the patient is preferably a human. DME can affect patients of all ages. Accordingly, the human patient can be a child (ages 0 to 18 years) or an adult (18 years old or older).

### Active Ingredient

The active ingredient is a plasma kallikrein inhibitor, which is the compound of Formula A:

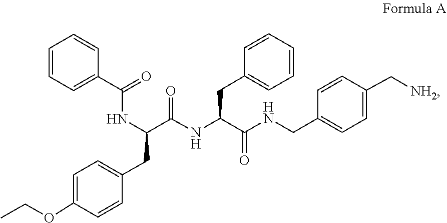 [SMILES: CCOc1ccc(C[C@@H](NC(=O)c2ccccc2)C(=O)N[C@@H](Cc2ccccc2)C(=O)NCc2ccc(CN)cc2)cc1]

The compound of Formula A is N—[(R)-1-[(S)-1-(4-Aminomethyl-benzylcarbamoyl)-2-phenyl-ethylcarbamoyl]-2-(4-ethoxy-phenyl)-ethyl]-benzamide.

The compound of Formula A may also be referred to as N—((R)-1-(((S)-1-((4-(aminomethyl)benzyl)amino)-1-oxo-3-phenylpropan-2-yl)amino)-3-(4-ethoxyphenyl)-1-oxopropan-2-yl)benzamide.

### Solid Forms

The invention is to be understood not to be limited by the identity of the solid compound of Formula A used to prepare the formulation for administration. Moreover, the formulation of the compound of Formula A involves formulation as a solution, and as such the identity of the solid form used to prepare said solution has no bearing on the invention; the free base of the compound of Formula A is the active ingredient. The hydrochloride salt solid form is used in the preparative methods in the examples of the present invention. However, it is within the scope of the present invention to use any solid form, including any solid form of any salt, solvate, or hydrate to prepare the formulation of the compound of Formula A. It is also within the scope of the present invention to use any solid form of any salt and/or solvate (i.e. salt, solvate or solvate of a salt) to prepare the formulation of the compound of Formula A.

### Uses and Methods

The uses and methods of the invention involve intravitreal administration of the compound of Formula A. Preferably, the uses and methods of the invention involve intravitreal administration of a pharmaceutical composition comprising the compound of Formula A. More preferably, the pharmaceutical composition comprising the compound of Formula A is an aqueous solution comprising the compound of Formula A.

As used herein, any reference to the compound of Formula A in its administration, i.e. in a formulation, composition, or pharmaceutical composition, refers to the administration of the compound of Formula A for use in the uses and the methods of the invention as outlined above.

The uses and methods preferably involve administration of a pharmaceutical composition, in particular aqueous solutions. Preferably, the pharmaceutical composition meets the requirements of USP <788> (Particulate matter in injections) for a small-volume injection with a container volume of 2 mL when measured using the microscopic particle count test. The acceptance limits provided in USP <788> for a small-volume injection using the microscopic particle count test are that the number of particles present (actual or calculated) in each discrete unit tested or in each pooled sample tested does not exceed 3000 per container equal to or greater than 10 μm, and does not exceed 300 per container equal to or greater than 25 μm.

More preferably, the pharmaceutical composition meets the requirements of USP <788> (Particulate matter in injections) for a large-volume injection when measured using the microscopic particle count test. The acceptance limits provided in USP <788> for a large-volume injection using the microscopic particle count test are that the number of particles present (actual or calculated) in each discrete unit tested or in each pooled sample tested does not exceed 12 per mL equal to or greater than 10 μm, and does not exceed 2 per mL equal to or greater than 25 μm.

More preferably, the pharmaceutical composition meets the requirements of USP <789> (Particulate matter in ophthalmic solutions) when measured using the microscopic particle count test. The acceptance limits provided in USP <789> using the microscopic particle count test are that the average number of particles present in the units tested does not exceed 50 per mL equal to or greater than 10 μm, and does not exceed 5 per mL equal to or greater than 25 μm, and does not exceed 2 per mL equal to or greater than 50 μm.

The references to USP <788> and USP <789> herein refer to USP <788> and USP <789> in United States Pharmacopeia (USP) 37, NF 32.

The compositions can be aqueous. However, the compositions can be pre-formulated as a sterile, non-aqueous solution or in a dried form which can be subsequently reconstituted with a suitable aqueous vehicle (e.g. sterile, pyrogen-free water). The composition may be provided as a bulk solution which is further diluted, for example with sterile, pyrogen-free water, prior to use.

The compositions may be hypotonic, isotonic or hypertonic. The compositions typically have an osmolality of from about 250 to about 350 mOsmol/kg. For example, the compositions may have an osmolality of 250, 260, 270, 280, 290, 300, 310, 320, 330, 340 or 350 mOsmol/kg.

The compositions will typically be at a pH of from about 2 to about 10, e.g. pH 2, 3, 4, 5, 6, 7, 8, 9 or 10. The pH of the composition is preferably from about 2 to about 10, more preferably from about 5 to about 7.5, even more preferably from about 5.3 to about 6, yet more preferably from about 5.4 to about 5.8 and most preferably about 5.5.

Typically, the active ingredient, i.e. the compound of Formula A, is present in the composition at a concentration of from about 10 μg/mL to about 300 μg/mL, or from about 10 μg/mL to about 250 μg/mL, or from about 10 μg/mL to about 200 μg/mL, or from about 20 μg/mL to about 200 μg/mL, or from about 20 μg/mL to about 160 μg/mL, or from about 20 μg/mL to about 120 μg/mL, or from about 20 μg/mL to about 100 μg/mL. In a preferred embodiment the active ingredient, i.e. the compound of formula A, is present in the composition at a concentration of from about 30 μg/mL to about 100 μg/mL. Also, preferably the active ingredient, i.e. the compound of Formula A, is present in the composition of from about 30 μg/mL to about 60 μg/mL. More preferably, the active ingredient, i.e. the compound of Formula A, is present in the composition of from about 60 μg/mL to about 100 μg/mL Typically, the active ingredient, i.e. the compound of Formula A, is present in the composition at a concentration of about 30 μg/mL, about 60 μg/mL, about 100 μg/mL, about 120 μg/mL, or about 200 μg/mL, or about 250 μg/mL, or about 300 μg/mL. The concentrations specified refer to the concentration of the free base of the compound of Formula A in the composition. The free base of the compound of Formula A has the structure depicted in Formula A.

The concentration of the compound of Formula A is the concentration as administered. For example, this is the concentration of the compound of Formula A at the point at which it is administered to a patient. In particular, it is the concentration at the point that it is intravitreally injected. The formulation of the compound of Formula A is generally a pharmaceutical composition. The pharmaceutical composition is a solution, which may be preferably an aqueous solution.

The compound of Formula A used in the invention may be isolated in the form of its pharmaceutically acceptable salts, such as those described herein. The pharmaceutically acceptable salt is typically a hydrochloride salt.

### Excipients

The compound of Formula A may be intravitreally administered, i.e. injection into the eye. The compound of Formula A may be provided with one or more pharmaceutically acceptable excipients. The term ‘excipient’ is used herein to describe any ingredient other than the active ingredient which may impart either a functional (e.g. injectability, stability enhancing, drug release rate controlling) and/or a non-functional (e.g. processing aid or diluent) characteristic to the formulations. The choice of excipient will to a large extent depend on factors such as the particular mode of administration, the effect of the excipient on solubility and stability, and the nature of the dosage form.

The compound of Formula A may be provided with at least one buffer. The use of a buffer can minimize fluctuations in pH, which may improve stability and/or improve the tolerability of the composition in a subject upon administration. Suitable buffers that can be used in the compositions of the invention include histidine, acetate, citrate, cacodylate, bis-tris, maleate, piperazine, MES (2-(N-morpholino)ethanesulfonic acid), tartrate, lactate; succinate; sulfate; phosphate; alanine; imidazole; arginine and asparagine. Typically, the buffer is selected from histidine, maleate and citrate. Preferably, the buffer is histidine. The pH of the buffer will typically be between about 2 and about 10, e.g. about pH 2, 3, 4, 5, 6, 7, 8, 9 or 10. The pH of the buffer is preferably from about 2 to about 10, more preferably from about 5 to about 7.5, even more preferably from about 5.3 to about 6, yet more preferably from about 5.4 to about 5.8 and most preferably about 5.5.

The pH of the buffer may be adjusted by the addition of an acid or a base. For example, the pH of the buffer may be adjusted with hydrochloric acid. The buffers referred to are also intended to include salts of the buffer. For example, histidine buffer includes histidine hydrochloride buffer.

The compound of Formula A may be administered with a buffer in an amount from about 0.0001% to about 1%, or from about 0.001% to about 0.32%, optionally from about 0.01% to about 0.16%. The compound of Formula A may be administered with a buffer in an amount from about 0.01% to about 0.08% by weight of the composition. Typically, the compound of Formula A may be administered with a buffer in an amount of about 0.01%, 0.02%, 0.03% or 0.04% by weight of the composition.

The compound of Formula A may be administered with at least one non-ionic tonicity agent. The use of a non-ionic tonicity agent can aid control of the osmolality of the composition. The non-ionic tonicity agent is typically a carbohydrate and is preferably a sugar. The non-ionic tonicity agent may be selected from the group comprising glycerine; sugars, e.g. glucose, mannitol, sorbitol, trehalose, dextrose, lactose, maltose, fructose, sucrose, and inositol; hydroxyethyl starch, e.g. hetastarch and pentastarch. The non-ionic tonicity agent is typically trehalose. Preferably, the non-ionic tonicity agent is trehalose.

The compound of Formula A may be administered histidine as the buffer and trehalose as the non-ionic tonicity agent.

The compound of Formula A may be administered as a hypotonic, isotonic or hypertonic formulation. It may be desirable that a formulation for intravitreal injection is isotonic to the vitreous, i.e. has the same effective osmolality as the vitreous, so as not to disrupt the fluid balance of the vitreous and surrounding tissues.

The compound of Formula A may be administered with a non-ionic tonicity agent in an amount from about 0.1% to about 30% by weight of the composition, e.g. about 0.1%, 0.2%, 0.3%, 0.4%, 0.5%, 0.6%, 0.7%, 0.8%, 0.9%, 1%, 2.5%, 5%, 10%, 15%, 20%, 25% or 30% by weight of the composition. The compound of Formula A may be administered with a non-ionic tonicity agent in an amount from about 1% to about 20%, or from about 5% to about 15%, or from about 7% to about 12% by weight of the composition, or from about 8% to about 10% by weight of the composition. Typically, the compound of Formula A may be administered with a non-ionic tonicity agent in an amount of about 8%, 9% or 10% by weight of the composition.

The compound of Formula A may be administered in a formulation with an osmolality of from about 250 to about 350 mOsmol/kg. For example, the formulations may have an osmolality of 250, 260, 270, 280, 290, 300, 310, 320, 330, 340 or 350 mOsmol/kg. The skilled person will understand that the amount of non-ionic tonicity agent used may vary depending on the particular choice of agent and on the other components in the composition.

The compound of Formula A may be administered with a non-ionic surfactant, such as carboxylic esters, polyethylene glycol esters, glycol esters of fatty acids, ethoxylated aliphatic alcohols, polyoxyethelene surfactants, sorbitol esters, ethoxylated derivatives of sorbitol esters, glycol esters of fatty acids, and poloxamers. Polyoxyethelene surfactants include polyoxyethylenesorbitan fatty acid esters, which are also referred to as polysorbates, e.g. polysorbate 80 (polyoxyethylene sorbitan monooleate, Tween® 80), polysorbate 40 (polyoxyethylene sorbitan monopalmitate, Tween® 40) and polysorbate 20 (polyoxyethylene sorbitan monolaurate, Tween® 20). Preferably, the non-ionic surfactant is a polyoxyethylenesorbitan fatty acid ester. More preferably, the non-ionic surfactant is polysorbate 20.

Alternatively, the compound of Formula A may be administered in a formulation that is free, or substantially free, of non-ionic surfactants, such as carboxylic esters, polyethylene glycol esters, glycol esters of fatty acids, ethoxylated aliphatic alcohols, polyoxyethelene surfactants, sorbitol esters, ethoxylated derivatives of sorbitol esters, glycol esters of fatty acids, and poloxamers. Polyoxyethelene surfactants include polyoxyethylenesorbitan fatty acid esters, which are also referred to as polysorbates, e.g. polysorbate 80 (polyoxyethylene sorbitan monooleate, Tween® 80), polysorbate 40 (polyoxyethylene sorbitan monopalmitate, Tween® 40) and polysorbate 20 (polyoxyethylene sorbitan monolaurate, Tween® 20). The compositions of the invention are preferably free of polysorbate, e.g. polysorbate 20.

The compound of Formula A may be administered with histidine as the buffer and trehalose as the non-ionic tonicity agent and may optionally be free, or substantially free, of polysorbate, e.g. polysorbate 20.

The compound of Formula A may be administered with an antioxidant, such as acetone, sodium bisulfite, butylated hydroxy anisole, butylated hydroxy toluene, cysteine, cysteinate HCl, dithionite sodium, gentisic acid, gentisic acid ethanolamine, glutamate monosodium, formaldehyde sulfoxylate sodium, metabisulfite potassium, metabisulfite sodium, monothioglycerol, propyl gallate, sulfite sodium, thioglycolate sodium or ascorbic acid. Alternatively, in particular for intraocular use of the composition, packaging may be configured in a manner that controls the potential for oxidation of the composition, including for example purging with an inert gas during manufacture.

The compound of Formula A may be formulated in any method suitable for intravitreal administration, for example in the formulations described above. These formulations may be prepared by standard procedures that would be well known and understood by the person skilled in the art.

### Preparations

The compound of Formula A may be formulated by a method involving the steps of:
a) preparing a solution of at least one non-ionic tonicity agent and at least one buffer in water;
b) dissolving a compound of Formula A, or a pharmaceutically acceptable salt thereof, in the solution prepared in step (a);
   wherein the at least one non-ionic tonicity agent, the at least one buffer, and the compound of formula A are as defined herein.

Preferably, the water used in step (a) is sterile water for injection.

The method may further comprise the step of:
(c) adding an aqueous solution of at least one non-ionic tonicity agent and at least one buffer to the solution prepared in step (b); and/or
(d) sterilising the solution.

Preferably, the sterilisation in step (d) is performed by sterile filtration.

A further method for preparing the formulations suitable for use in the invention, comprises adding water to a non-aqueous formulation comprising at least one non-ionic tonicity agent, at least one buffer and an active ingredient, wherein said active ingredient is a compound of Formula A or a pharmaceutically acceptable salt thereof, and wherein the at least one non-ionic tonicity agent, the at least one buffer, and the compound of Formula A are as defined herein.

Any solid form of the compound of Formula A may be used in preparing the formulation. The formulation may be provided as a solution formulation.

It would be readily understood that the invention is not limited to the use of specific solid forms. Any other solid form could also be used to prepare solution formulations of the compound of Formula A.

### Definitions

The term “aqueous” means that the composition includes water as a solvent. Typically, the content of water in the composition is greater than or equal to about 35% by weight, preferably more than about 50% by weight of the composition, e.g. more than about 60%, 65%, 70%, 75%, 80%, 85%, 90%, 95%, 97%, 98% or 99% by weight of the composition.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical quantities x (excluding measurements of time) is optional and means, for example, x±10%.

The term “about” in relation to measurements of weeks is optional and means, for example “4 weeks ±1 week”. More specifically, the term “about” in relation to a measurements of weeks is optional and means, for example “4 weeks ±3 days”.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

Where compounds used in the compositions of the invention exist in one or more geometrical, optical, enantiomeric, diastereomeric and tautomeric forms, including but not limited to cis- and trans-forms, E- and Z-forms, R-, S- and meso-forms, keto-, and enol-forms, then, unless otherwise stated, a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

A reference to a particular compound also includes all isotopic variants, including deuterated variants.

In the context of the present invention, references herein to “treatment” include references to curative, palliative, prophylaxis, a prevention of worsening of the condition/indication/disease, a protective treatment, a slowing of the progression of the condition/indication/disease, or a slowing of the onset of the condition/indication/disease. The noun “treatment” may be used interchangeably with the verb “to treat”, with the same meaning.

“anti-VEGF treatment” and “anti-VEGF therapy” may be used interchangeably throughout. Anti-VEGF treatment comprises any treatment that comprises administration of an anti-vascular endothelial growth factor. Examples of such anti-VEGF therapies include the use of aflibercept (Eylea®), bevacizumab, ranibizumab and pegaptanib. Anti-VEGF treatment, as used herein refers to anti-VEGF therapy for use in the treatment of any condition. Particularly, anti-VEGF therapy refers to anti-VEGF therapy for use in treatment of any condition by intravitreal injection. Preferably, anti-VEGF therapy refers to anti-VEGF therapy for DME, or impaired visual acuity.

“AE” as used herein refers to adverse events, and has the usual clinical meaning that would be readily understood by the person skilled in the art.

The term “Diabetic macular edema” or “DME” would be readily understood by the skilled person, and includes all types of DME. DME may be used interchangeably with the term center-involving DME (ciDME). “Edema” may also be referred to as “Oedema”, and both terms may be used interchangeably throughout.

The term “impaired visual acuity” encompasses any medical condition whose symptoms involve a decrease in visual acuity. For example, said impaired visual acuity may be measured by Best Corrected Visual Acuity (BCVA) with the Early Treatment of Diabetic Retinopathy Study. Examples of conditions with symptoms of impaired visual acuity include diabetic macular edema, diabetic retinopathy, retinal vascular permeability associated with diabetic retinopathy, retinal vascular occlusion, diabetes, macular degeneration and neuropathy.

As used herein, a number of characteristic indicators may be used to assess the symptoms of DME or impaired visual acuity. For example, visual acuity, which may assessed as Best Corrected Visual Acuity (BCVA), measured by the standard Early Treatment Diabetic Retinopathy Study (ETDRS) chart, values can indicate DME or impaired visual acuity. Presence of DME or impaired visual acuity is measured in patients at baseline. For example, a BCVA score of ≥19 and ≤73 letters in the eye which is implicated may be symptomatic of DME or impaired visual acuity.

Alternatively, a BCVA score 19 and 55 letters may be symptomatic of DME or impaired visual acuity.

The “early stages of DME” may be defined by patients who, at baseline, have a BCVA score of ≥56 and ≤73. This may also be referred to interchangeably as “early onset of DME”. The “early stages of impaired visual acuity” may be defined by patients who, at baseline, have a BCVA score of ≥56 and ≤73. This may also be referred to interchangeably as “early onset of impaired visual acuity”.

The “compound of Formula A” as used herein is to be understood to refer to the “compound of Formula A (or a pharmaceutically acceptable salt and/or solvate thereof)”.

The term “baseline”, in reference to any measurement of a value, refers to the measurement of that value before any treatment has commenced.

“μg” refers to the measurement of micrograms, and may be used interchangeably with “ug”.

The term “dosing frequency” refers to the number of doses given in a unit period of time. Therefore, a reduced or lower dosing frequency refers to any of:
fewer doses in the same given time period;
the same number of doses in a longer time period;
fewer doses in a longer time period.

The term “dose” may be used interchangeably with any reference to “an intravitreal administration”, which can, for example, refer to an intravitreal injection.

As used herein, visual acuity scores are measured as a Best Corrected Visual Acuity (BCVA) using the standard Early Treatment Diabetic Retinopathy Study (ETDRS) chart. The procedure for measuring BCVA using the ETDRS chart is outlined in Ophthalmology 1991; 98:741-756, with reference to Ferris F L III et al “New visual acuity charts for clinical research” Am J Ophthalmol 1982; 94:91-6.

### Exemplary Numbered Embodiments

1. A method for treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

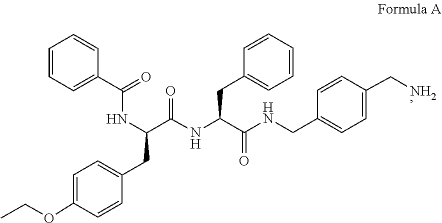 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.
2. The compound of Formula A (or a pharmaceutically acceptable salt and/or solvate thereof) for use in treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

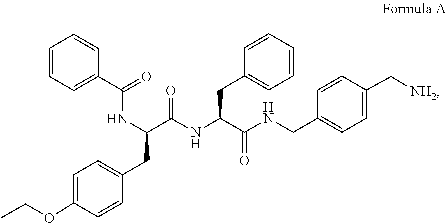 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.
3. The use of the compound of Formula A (or a pharmaceutically acceptable salt and/or solvate thereof) in the manufacture of a medicament for the treatment of diabetic macular edema (DME), comprising: intravitreally administering a pharmaceutical composition, wherein the pharmaceutical composition is a solution comprising the compound of Formula A (or a pharmaceutically acceptable salt and/or solvate thereof), to a patient in need thereof,

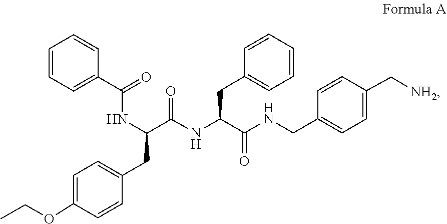 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.
4. A method for treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

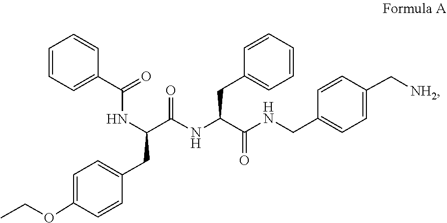 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.
5. The compound of Formula A (or a pharmaceutically acceptable salt and/or solvate thereof) for use in treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

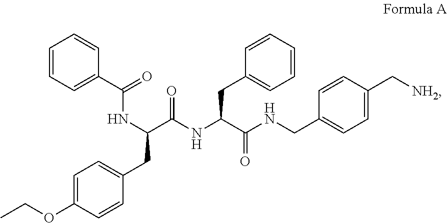 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.
6. The use of the compound of Formula A (or a pharmaceutically acceptable salt and/or solvate thereof) in the manufacture of a medicament for the treatment of impaired visual acuity, comprising: intravitreally administering a pharmaceutical composition, wherein the pharmaceutical composition is a solution comprising the compound of Formula A (or a pharmaceutically acceptable salt and/or solvate thereof), to a patient in need thereof,

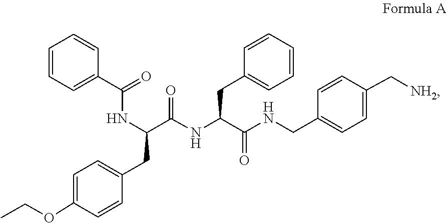 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient has previously had anti-VEGF (vascular endothelial growth factor) treatment.
7. The method according to any one of embodiments 1 or 4, the compound of Formula A (or a pharmaceutically acceptable salt and/or solvate thereof) for use according to any one of embodiments 2 or 5, or the use of the compound of Formula A (or a pharmaceutically acceptable salt and/or solvate thereof) according to any one of embodiments 3 or 6,
   wherein the pharmaceutical composition is an aqueous solution comprising the compound of Formula A (or a pharmaceutically acceptable salt and/or solvate thereof).
8. The method according to any one of embodiments 1, 4 or 7, the compound of Formula A (or a pharmaceutically acceptable salt and/or solvate thereof) for use according to any one of embodiments 2, 5 or 7, or the use of the compound of Formula A (or a pharmaceutically acceptable salt and/or solvate thereof) according to any one of embodiments 3, 6 or 7,
   wherein intravitreal administration comprises intravitreal injection.
9. The method according to any one of embodiments 1, 4 or 7-8, the compound of Formula A (or a pharmaceutically acceptable salt and/or solvate thereof) for use according to any one of embodiments 2, 5 or 7-8, or the use of the compound of Formula A (or a pharmaceutically acceptable salt and/or solvate thereof) according to any one of embodiments 3, 6 or 7-8,
   wherein the pharmaceutical composition is intravitreally administered into at least one of the patient's eye.
10. The method according to any one of embodiments 1, 4 or 7-9, the compound of Formula A (or a pharmaceutically acceptable salt and/or solvate thereof) for use according to any one of embodiments 2, 5 or 7-9, or the use of the compound of Formula A (or a pharmaceutically acceptable salt and/or solvate thereof) according to any one of embodiments 3, 6 or 7-9,
   wherein the pharmaceutical composition is intravitreally administered into both of the patient's eyes.
11. The method according to any one of embodiments 1, 4 or 7-10, the compound of Formula A (or a pharmaceutically acceptable salt and/or solvate thereof) for use according to any one of embodiments 2, 5 or 7-10, or the use of the compound of Formula A (or a pharmaceutically acceptable salt and/or solvate thereof) according to any one of embodiments 3, 6 or 7-10,
   wherein the solution further comprises at least one non-ionic tonicity agent.
12. The method according to embodiment 11, the compound of Formula A (or a pharmaceutically acceptable salt and/or solvate thereof) for use according to embodiment 11, or the use of the compound of Formula A (or a pharmaceutically acceptable salt and/or solvate thereof) according to embodiment 11,
   wherein the at least one non-ionic tonicity agent is trehalose.
13. The method according to embodiment 12, the compound of Formula A (or a pharmaceutically acceptable salt and/or solvate thereof) for use according to embodiment 12, or the use of the compound of Formula A (or a pharmaceutically acceptable salt and/or solvate thereof) according to embodiment 12,
   wherein the trehalose is provided as trehalose dihydrate.
14. The method according to any one of embodiments 1, 4 or 7-13, the compound of Formula A (or a pharmaceutically acceptable salt and/or solvate thereof) for use according to any one of embodiments 2, 5 or 7-13, or the use of the compound of Formula A (or a pharmaceutically acceptable salt and/or solvate thereof) according to any one of embodiments 3, 6 or 7-13,
   wherein the solution further comprises histidine.
15. The method according to any one of embodiments 1, 4 or 7-14, the compound of Formula A (or a pharmaceutically acceptable salt and/or solvate thereof) for use according to any one of embodiments 2, 5 or 7-14, or the use of the compound of Formula A (or a pharmaceutically acceptable salt and/or solvate thereof) according to any one of embodiments 3, 6 or 7-14,
   wherein the pharmaceutical composition comprises an aqueous solution of the compound of formula A (or a pharmaceutically acceptable salt and/or solvate thereof), histidine and trehalose dihydrate.
16. The method according to any one of embodiments 1, 4 or 7-15, the compound of Formula A (or a pharmaceutically acceptable salt and/or solvate thereof) for use according to any one of embodiments 2, 5 or 7-15, or the use of the compound of Formula A (or a pharmaceutically acceptable salt and/or solvate thereof) according to any one of embodiments 3, 6 or 7-15,
   wherein the pharmaceutical composition has a pH from about 2 to about 10, preferably from about 5 to about 7.5, preferably from about 5.3 to about 6, and preferably from about 5.4 to about 5.8.
17. The method according to any one of embodiments 1, 4 or 7-16, the compound of Formula A (or a pharmaceutically acceptable salt and/or solvate thereof) for use according to any one of embodiments 2, 5 or 7-16, or the use of the compound of Formula A (or a pharmaceutically acceptable salt and/or solvate thereof) according to any one of embodiments 3, 6 or 7-16,
   wherein the pharmaceutical composition has a pH of about 5.5.
18. The method according to any one of embodiments 1, 4 or 7-17, the compound of Formula A (or a pharmaceutically acceptable salt and/or solvate thereof) for use according to any one of embodiments 2, 5 or 7-17, or the use of the compound of Formula A (or a pharmaceutically acceptable salt and/or solvate thereof) according to any one of embodiments 3, 6 or 7-17,
   wherein the concentration of the compound of Formula A (or a pharmaceutically acceptable salt and/or solvate thereof) when administered is between about 10 μg/mL and about 300 μg/mL based on the concentration of the free base of the compound of Formula A in solution.
19. The method according to any one of embodiments 1, 4 or 7-18, the compound of Formula A (or a pharmaceutically acceptable salt and/or solvate thereof) for use according to any one of embodiments 2, 5 or 7-18, or the use of the compound of Formula A (or a pharmaceutically acceptable salt and/or solvate thereof) according to any one of embodiments 3, 6 or 7-18,
   wherein the concentration of the compound of Formula A (or a pharmaceutically acceptable salt and/or solvate thereof) when administered is between about 10 μg/mL and about 250 μg/mL based on the concentration of the free base of the compound of Formula A in solution.
20. The method according to any one of embodiments 1, 4 or 7-19, the compound of Formula A (or a pharmaceutically acceptable salt and/or solvate thereof) for use according to any one of embodiments 2, 5 or 7-19, or the use of the compound of Formula A (or a pharmaceutically acceptable salt and/or solvate thereof) according to any one of embodiments 3, 6 or 7-19,
   wherein the concentration of the compound of Formula A (or a pharmaceutically acceptable salt and/or solvate thereof) when administered is between about 10 μg/mL and about 200 μg/mL based on the concentration of the free base of the compound of Formula A in solution.
21. The method according to any one of embodiments 1, 4 or 7-20, the compound of Formula A (or a pharmaceutically acceptable salt and/or solvate thereof) for use according to any one of embodiments 2, 5 or 7-20, or the use of the compound of Formula A (or a pharmaceutically acceptable salt and/or solvate thereof) according to any one of embodiments 3, 6 or 7-20,
   wherein the concentration of the compound of Formula A (or a pharmaceutically acceptable salt and/or solvate thereof) when administered is between about 20 μg/mL and about 200 μg/mL based on the concentration of the free base of the compound of Formula A in solution.
22. The method according to any one of embodiments 1, 4 or 7-21, the compound of Formula A (or a pharmaceutically acceptable salt and/or solvate thereof) for use according to any one of embodiments 2, 5 or 7-21, or the use of the compound of Formula A (or a pharmaceutically acceptable salt and/or solvate thereof) according to any one of embodiments 3, 6 or 7-21,
   wherein the concentration of the compound of Formula A (or a pharmaceutically acceptable salt and/or solvate thereof) when administered is between about 20 μg/mL and about 160 μg/mL based on the concentration of the free base of the compound of Formula A in solution.
23. The method according to any one of embodiments 1, 4 or 7-22, the compound of Formula A (or a pharmaceutically acceptable salt and/or solvate thereof) for use according to any one of embodiments 2, 5 or 7-22, or the use of the compound of Formula A (or a pharmaceutically acceptable salt and/or solvate thereof) according to any one of embodiments 3, 6 or 7-22,
   wherein the concentration of the compound of Formula A (or a pharmaceutically acceptable salt and/or solvate thereof) when administered is between about 20 μg/mL and about 120 μg/mL based on the concentration of the free base of the compound of Formula A in solution.
24. The method according to any one of embodiments 1, 4 or 7-23, the compound of Formula A (or a pharmaceutically acceptable salt and/or solvate thereof) for use according to any one of embodiments 2, 5 or 7-23, or the use of the compound of Formula A (or a pharmaceutically acceptable salt and/or solvate thereof) according to any one of embodiments 3, 6 or 7-23,
   wherein the concentration of the compound of Formula A (or a pharmaceutically acceptable salt and/or solvate thereof) when administered is between about 20 μg/mL and about 100 μg/mL based on the concentration of the free base of the compound of Formula A in solution.
25. The method according to any one of embodiments 1, 4 or 7-24, the compound of Formula A (or a pharmaceutically acceptable salt and/or solvate thereof) for use according to any one of embodiments 2, 5 or 7-24, or the use of the compound of Formula A (or a pharmaceutically acceptable salt and/or solvate thereof) according to any one of embodiments 3, 6 or 7-24,
   wherein the concentration of the compound of Formula A (or a pharmaceutically acceptable salt and/or solvate thereof) when administered is between about 30 μg/mL and about 100 μg/mL based on the concentration of the free base of the compound of Formula A in solution.
26. The method according to any one of embodiments 1, 4 or 7-25, the compound of Formula A (or a pharmaceutically acceptable salt and/or solvate thereof) for use according to any one of embodiments 2, 5 or 7-25, or the use of the compound of Formula A (or a pharmaceutically acceptable salt and/or solvate thereof) according to any one of embodiments 3, 6 or 7-25,
   wherein the concentration of the compound of Formula A (or a pharmaceutically acceptable salt and/or solvate thereof) when administered is between about 60 μg/mL and about 100 μg/mL based on the concentration of the free base of the compound of Formula A in solution.
27. The method according to any one of embodiments 1, 4 or 7-25, the compound of Formula A (or a pharmaceutically acceptable salt and/or solvate thereof) for use according to any one of embodiments 2, 5 or 7-25, or the use of the compound of Formula A (or a pharmaceutically acceptable salt and/or solvate thereof) according to any one of embodiments 3, 6 or 7-25,
   wherein the concentration of the compound of Formula A (or a pharmaceutically acceptable salt and/or solvate thereof) when administered is about 30 μg/mL based on the concentration of the free base of the compound of Formula A in solution.
28. The method according to any one of embodiments 1, 4 or 7-26, the compound of Formula A (or a pharmaceutically acceptable salt and/or solvate thereof) for use according to any one of embodiments 2, 5 or 7-26, or the use of the compound of Formula A (or a pharmaceutically acceptable salt and/or solvate thereof) according to any one of embodiments 3, 6 or 7-26,
   wherein the concentration of the compound of Formula A (or a pharmaceutically acceptable salt and/or solvate thereof) when administered is about 60 μg/mL based on the concentration of the free base of the compound of Formula A in solution.
29. The method according to any one of embodiments 1, 4 or 7-26, the compound of Formula A (or a pharmaceutically acceptable salt and/or solvate thereof) for use according to any one of embodiments 2, 5 or 7-26, or the use of the compound of Formula A (or a pharmaceutically acceptable salt and/or solvate thereof) according to any one of embodiments 3, 6 or 7-26,
   wherein the concentration of the compound of Formula A (or a pharmaceutically acceptable salt and/or solvate thereof) when administered is about 100 μg/mL based on the concentration of the free base of the compound of Formula A in solution.
30. The method according to any one of embodiments 1, 4 or 7-23, the compound of Formula A (or a pharmaceutically acceptable salt and/or solvate thereof) for use according to any one of embodiments 2, 5 or 7-23, or the use of the compound of Formula A (or a pharmaceutically acceptable salt and/or solvate thereof) according to any one of embodiments 3, 6 or 7-23,
   wherein the concentration of the compound of Formula A (or a pharmaceutically acceptable salt and/or solvate thereof) when administered is about 120 μg/mL based on the concentration of the free base of the compound of Formula A in solution.
31. The method according to any one of embodiments 1, 4 or 7-21, the compound of Formula A (or a pharmaceutically acceptable salt and/or solvate thereof) for use according to any one of embodiments 2, 5 or 7-21, or the use of the compound of Formula A (or a pharmaceutically acceptable salt and/or solvate thereof) according to any one of embodiments 3, 6 or 7-21,
   wherein the concentration of the compound of Formula A (or a pharmaceutically acceptable salt and/or solvate thereof) when administered is about 200 μg/mL based on the concentration of the free base of the compound of Formula A in solution.
32. The method according to any one of embodiments 1, 4 or 7-31, the compound of Formula A (or a pharmaceutically acceptable salt and/or solvate thereof) for use according to any one of embodiments 2, 5 or 7-31, or the use of the compound of Formula A (or a pharmaceutically acceptable salt and/or solvate thereof) according to any one of embodiments 3, 6 or 7-31,
   wherein about 10 μL to about 100 μL of the solution is administered per intravitreal administration.
33. The method according to any one of embodiments 1, 4 or 7-32, the compound of Formula A (or a pharmaceutically acceptable salt and/or solvate thereof) for use according to any one of embodiments 2, 5 or 7-32, or the use of the compound of Formula A (or a pharmaceutically acceptable salt and/or solvate thereof) according to any one of embodiments 3, 6 or 7-32,
   wherein about 25 μL to about 100 μL of the solution is administered per intravitreal administration.
34. The method according to any one of embodiments 1, 4 or 7-33, the compound of Formula A (or a pharmaceutically acceptable salt and/or solvate thereof) for use according to any one of embodiments 2, 5 or 7-33, or the use of the compound of Formula A (or a pharmaceutically acceptable salt and/or solvate thereof) according to any one of embodiments 3, 6 or 7-33,
   wherein about 50 μL to about 100 μL of the solution is administered per intravitreal administration.
35. The method according to any one of embodiments 1, 4 or 7-34, the compound of Formula A (or a pharmaceutically acceptable salt and/or solvate thereof) for use according to any one of embodiments 2, 5 or 7-34, or the use of the compound of Formula A (or a pharmaceutically acceptable salt and/or solvate thereof) according to any one of embodiments 3, 6 or 7-34,
   wherein about 50 μL to about 60 μL of the solution is administered per intravitreal administration.
36. The method according to any one of embodiments 1, 4 or 7-34, the compound of Formula A (or a pharmaceutically acceptable salt and/or solvate thereof) for use according to any one of embodiments 2, 5 or 7-34, or the use of the compound of Formula A (or a pharmaceutically acceptable salt and/or solvate thereof) according to any one of embodiments 3, 6 or 7-34,
   wherein about 60 μL to about 70 μL of the solution is administered per intravitreal administration.
37. The method according to any one of embodiments 1, 4 or 7-34, the compound of Formula A (or a pharmaceutically acceptable salt and/or solvate thereof) for use according to any one of embodiments 2, 5 or 7-34, or the use of the compound of Formula A (or a pharmaceutically acceptable salt and/or solvate thereof) according to any one of embodiments 3, 6 or 7-34,
   wherein about 70 μL to about 80 μL of the solution is administered per intravitreal administration.
38. The method according to any one of embodiments 1, 4 or 7-34, the compound of Formula A (or a pharmaceutically acceptable salt and/or solvate thereof) for use according to any one of embodiments 2, 5 or 7-34, or the use of the compound of Formula A (or a pharmaceutically acceptable salt and/or solvate thereof) according to any one of embodiments 3, 6 or 7-34,
   wherein about 80 μL to about 90 μL of the solution is administered per intravitreal administration.
39. The method according to any one of embodiments 1, 4 or 7-34, the compound of Formula A (or a pharmaceutically acceptable salt and/or solvate thereof) for use according to any one of embodiments 2, 5 or 7-34, or the use of the compound of Formula A (or a pharmaceutically acceptable salt and/or solvate thereof) according to any one of embodiments 3, 6 or 7-34,
   wherein about 90 μL to about 100 μL of the solution is administered per intravitreal administration.
40. The method according to any one of embodiments 1, 4, or 7-35, the compound of Formula A (or a pharmaceutically acceptable salt and/or solvate thereof) for use according to any one of embodiments 2, 5 or 7-35, or the use of the compound of Formula A (or a pharmaceutically acceptable salt and/or solvate thereof) according to any one of embodiments 3, 6 or 7-35,
   wherein about 50 μL of the solution is administered per intravitreal administration.
41. The method according to any one of embodiments 1, 4, or 7-36, the compound of Formula A (or a pharmaceutically acceptable salt and/or solvate thereof) for use according to any one of embodiments 2, 5 or 7-36, or the use of the compound of Formula A (or a pharmaceutically acceptable salt and/or solvate thereof) according to any one of embodiments 3, 6 or 7-36,
   wherein about 60 μL of the solution is administered per intravitreal administration.
42. The method according to any one of embodiments 1, 4, 7-34 or 36-37, the compound of Formula A (or a pharmaceutically acceptable salt and/or solvate thereof) for use according to any one of embodiments 2, 5, 7-34 or 36-37, or the use of the compound of Formula A (or a pharmaceutically acceptable salt and/or solvate thereof) according to any one of embodiments 3, 6, 7-34 or 36-37,
   wherein about 70 μL of the solution is administered per intravitreal administration.
43. The method according to any one of embodiments 1, 4, 7-34 or 37-38, the compound of Formula A (or a pharmaceutically acceptable salt and/or solvate thereof) for use according to any one of embodiments 2, 5, 7-34 or 37-38, or the use of the compound of Formula A (or a pharmaceutically acceptable salt and/or solvate thereof) according to any one of embodiments 3, 6, 7-34 or 37-38,
   wherein about 80 μL of the solution is administered per intravitreal administration.
44. The method according to any one of embodiments 1, 4, 7-34 or 38-39, the compound of Formula A (or a pharmaceutically acceptable salt and/or solvate thereof) for use according to any one of embodiments 2, 5, 7-34 or 38-39, or the use of the compound of Formula A (or a pharmaceutically acceptable salt and/or solvate thereof) according to any one of embodiments 3, 6, 7-34 or 38-39,
   wherein about 90 μL of the solution is administered per intravitreal administration.
45. The method according to any one of embodiments 1, 4, 7-34 or 39-40, the compound of Formula A (or a pharmaceutically acceptable salt and/or solvate thereof) for use according to any one of embodiments 2, 5, 7-34 or 39-40, or the use of the compound of Formula A (or a pharmaceutically acceptable salt and/or solvate thereof) according to any one of embodiments 3, 6, 7-34 or 39-40,
   wherein about 100 μL of the solution is administered per intravitreal administration.
46. The method according to any one of embodiments 1, 4 or 7-45, the compound of Formula A (or a pharmaceutically acceptable salt and/or solvate thereof) for use according to any one of embodiments 2, 5 or 7-45, or the use of the compound of Formula A (or a pharmaceutically acceptable salt and/or solvate thereof) according to any one of embodiments 3, 6 or 7-45,
   wherein prior to administration of the compound of Formula A, the patient has a baseline visual acuity score (BCVA) of between 19 and 73 letters in at least one eye, measured using the standard Early Treatment Diabetic Retinopathy Study (ETDRS) chart.
47. The method according to any one of embodiments 1, 4 or 7-46, the compound of Formula A (or a pharmaceutically acceptable salt and/or solvate thereof) for use according to any one of embodiments 2, 5 or 7-46, or the use of the compound of Formula A (or a pharmaceutically acceptable salt and/or solvate thereof) according to any one of embodiments 3, 6 or 7-46,
   wherein the patient is in the early stages of DME or impaired visual acuity.
48. The method according to embodiment 47, the compound of Formula A (or a pharmaceutically acceptable salt and/or solvate thereof) for use according to embodiment 47, or the use of the compound of Formula A (or a pharmaceutically acceptable salt and/or solvate thereof) according to embodiment 47,
   wherein a patient in the early stages of DME or impaired visual acuity is defined by having a baseline visual acuity score (BCVA), prior to administration of the compound of Formula A, of between 56 and 73 letters in at least one eye, measured using the standard Early Treatment Diabetic Retinopathy Study (ETDRS) chart.
49. The method according to any one of embodiments 1, 4 or 7-48, the compound of Formula A (or a pharmaceutically acceptable salt and/or solvate thereof) for use according to any one of embodiments 2, 5 or 7-48, or the use of the compound of Formula A (or a pharmaceutically acceptable salt and/or solvate thereof) according to any one of embodiments 3, 6 or 7-48,
   wherein the treatment is a monotherapy for DME or impaired visual acuity.
50. The method according to any one of embodiments 1, 4 or 7-49, the compound of Formula A (or a pharmaceutically acceptable salt and/or solvate thereof) for use according to any one of embodiments 2, 5 or 7-49, or the use of the compound of Formula A (or a pharmaceutically acceptable salt and/or solvate thereof) according to any one of embodiments 3, 6 or 7-49, wherein the anti-VEGF treatment is selected from aflibercept (Eylea®), bevacizumab, ranibizumab, and pegaptanib.
51. The method according to any one of embodiments 1, 4 or 7-50, the compound of Formula A (or a pharmaceutically acceptable salt and/or solvate thereof) for use according to any one of embodiments 2, 5 or 7-50, or the use of the compound of Formula A (or a pharmaceutically acceptable salt and/or solvate thereof) according to any one of embodiments 3, 6 or 7-50,
   wherein the anti-VEGF is aflibercept (Eylea®).
52. The method according to any one of embodiments 1, 4 or 7-50, the compound of Formula A (or a pharmaceutically acceptable salt and/or solvate thereof) for use according to any one of embodiments 2, 5 or 7-50, or the use of the compound of Formula A (or a pharmaceutically acceptable salt and/or solvate thereof) according to any one of embodiments 3, 6 or 7-50,
   wherein the patient received anti-VEGF treatment for no more than 36 months before commencing treatment with the compound of Formula A (or a pharmaceutically acceptable salt and/or solvate thereof).
53. The method according to any one of embodiments 1, 4 or 7-52, the compound of Formula A (or a pharmaceutically acceptable salt and/or solvate thereof) for use according to any one of embodiments 2, 5 or 7-52, or the use of the compound of Formula A (or a pharmaceutically acceptable salt and/or solvate thereof) according to any one of embodiments 3, 6 or 7-52,
   wherein the patient received anti-VEGF treatment no less than 8 weeks before commencing treatment with the compound of formula A (or a pharmaceutically acceptable salt and/or solvate thereof).
54. The method according to any one of embodiments 1, 4 or 7-53, the compound of Formula A (or a pharmaceutically acceptable salt and/or solvate thereof) for use according to any one of embodiments 2, 5 or 7-53, or the use of the compound of Formula A (or a pharmaceutically acceptable salt and/or solvate thereof) according to any one of embodiments 3, 6 or 7-53,
   wherein the patient does not receive anti-VEGF treatment concurrent to administration of the compound of Formula A.
55. The method according to any one of embodiments 1, 4 or 7-54, the compound of Formula A (or a pharmaceutically acceptable salt and/or solvate thereof) for use according to any one of embodiments 2, 5 or 7-54, or the use of the compound of Formula A (or a pharmaceutically acceptable salt and/or solvate thereof) according to any one of embodiments 3, 6 or 7-54,
   wherein the treatment is administered over a time period of at least about 12 weeks.
56. The method according to any one of embodiments 1, 4 or 7-55, the compound of Formula A (or a pharmaceutically acceptable salt and/or solvate thereof) for use according to any one of embodiments 2, 5 or 7-55, or the use of the compound of Formula A (or a pharmaceutically acceptable salt and/or solvate thereof) according to any one of embodiments 3, 6 or 7-55,
   wherein the treatment is administered at a first dosing frequency over a first time period, followed by a second dosing frequency over a second time period, wherein the second dosing frequency is lower than the first dosing frequency.
57. The method according to embodiment 56, the compound of Formula A (or a pharmaceutically acceptable salt and/or solvate thereof) for use according to embodiment 56, or the use of the compound of Formula A (or a pharmaceutically acceptable salt and/or solvate thereof) according to embodiment 56,
   wherein the first time period is greater than about 8 weeks.
58. The method according to any one of embodiments 56-57, the compound of Formula A (or a pharmaceutically acceptable salt and/or solvate thereof) for use according to any one of embodiments 56-57, or the use of the compound of Formula A (or a pharmaceutically acceptable salt and/or solvate thereof) according to any one of embodiments 56-57,
   wherein the first time period is greater than about 12 weeks.
59. The method according to any one of embodiments 56-57, the compound of Formula A (or a pharmaceutically acceptable salt and/or solvate thereof) for use according to any one of embodiments 56-57, or the use of the compound of Formula A (or a pharmaceutically acceptable salt and/or solvate thereof) according to any one of embodiments 56-57,
   wherein the first time period is between about 10 weeks and about 12 weeks.
60. The method according to any one of embodiments 56-59, the compound of Formula A (or a pharmaceutically acceptable salt and/or solvate thereof) for use according to any one of embodiments 56-59, or the use of the compound of Formula A (or a pharmaceutically acceptable salt and/or solvate thereof) according to any one of embodiments 56-59,
   wherein the first time period is about 12 weeks.
61. The method according to any one of embodiments 56-60, the compound of Formula A (or a pharmaceutically acceptable salt and/or solvate thereof) for use according to any one of embodiments 56-60, or the use of the compound of Formula A (or a pharmaceutically acceptable salt and/or solvate thereof) according to any one of embodiments 56-60,
   wherein the first dosing frequency is between about once every three weeks and about once every five weeks.
62. The method according to embodiment 61, the compound of Formula A (or a pharmaceutically acceptable salt and/or solvate thereof) for use according to embodiment 61, or the use of the compound of Formula A (or a pharmaceutically acceptable salt and/or solvate thereof) according embodiment 61,
   wherein the first dosing frequency is about once every four weeks.
63. The method according to any one of embodiments 56-62, the compound of Formula A (or a pharmaceutically acceptable salt and/or solvate thereof) for use according to any one of embodiments 56-62, or the use of the compound of Formula A (or a pharmaceutically acceptable salt and/or solvate thereof) according to any one of embodiments 56-62,
   wherein the second time period is greater than about 8 weeks.
64. The method according to any one of embodiments 56-63, the compound of Formula A (or a pharmaceutically acceptable salt and/or solvate thereof) for use according to any one of embodiments 56-63, or the use of the compound of Formula A (or a pharmaceutically acceptable salt and/or solvate thereof) according to any one of embodiments 56-63,
   wherein the second time period is greater than about 12 weeks.
65. The method according to any one of embodiments 56-64, the compound of Formula A (or a pharmaceutically acceptable salt and/or solvate thereof) for use according to any one of embodiments 56-64, or the use of the compound of Formula A (or a pharmaceutically acceptable salt and/or solvate thereof) according to any one of embodiments 56-64,
   wherein the second time period is greater than about 16 weeks.
66. The method according to any one of embodiments 56-63, the compound of Formula A (or a pharmaceutically acceptable salt and/or solvate thereof) for use according to any one of embodiments 56-63, or the use of the compound of Formula A (or a pharmaceutically acceptable salt and/or solvate thereof) according to any one of embodiments 56-63,
   wherein the second time period is between about 8 weeks and about 12 weeks.
67. The method according to any one of embodiments 56-63, the compound of Formula A (or a pharmaceutically acceptable salt and/or solvate thereof) for use according to any one of embodiments 56-63, or the use of the compound of Formula A (or a pharmaceutically acceptable salt and/or solvate thereof) according to any one of embodiments 56-63,
   wherein the second time period is about 12 weeks.
68. The method according to any one of embodiments 56-67, the compound of Formula A (or a pharmaceutically acceptable salt and/or solvate thereof) for use according to any one of embodiments 56-67, or the use of the compound of Formula A (or a pharmaceutically acceptable salt and/or solvate thereof) according to any one of embodiments 56-67,
   wherein the second dosing frequency is lower than about once every six weeks.
69. The method according to any one of embodiments 56-68, the compound of Formula A (or a pharmaceutically acceptable salt and/or solvate thereof) for use according to any one of embodiments 56-68, or the use of the compound of Formula A (or a pharmaceutically acceptable salt and/or solvate thereof) according to any one of embodiments 56-68,
   wherein the second dosing frequency is lower than about once every eight weeks.
70. The method according to any one of embodiments 56-69, the compound of Formula A (or a pharmaceutically acceptable salt and/or solvate thereof) for use according to any one of embodiments 56-69, or the use of the compound of Formula A (or a pharmaceutically acceptable salt and/or solvate thereof) according to any one of embodiments 56-69,
   wherein the second dosing frequency is lower than about once every twelve weeks.
71. The method according to any one of embodiments 1, 4 or 7-55, the compound of Formula A (or a pharmaceutically acceptable salt and/or solvate thereof) for use according to any one of embodiments 2, 5 or 7-55, or the use of the compound of Formula A (or a pharmaceutically acceptable salt and/or solvate thereof) according to any one of embodiments 3, 6 or 7-55,
   wherein the treatment is administered between about once every 4 weeks and once every 12 weeks.
72. The method according to any one of embodiments 1, 4, 7-55, or 71, the compound of Formula A (or a pharmaceutically acceptable salt and/or solvate thereof) for use according to any one of embodiments 2, 5, 7-55, or 71, or the use of the compound of Formula A (or a pharmaceutically acceptable salt and/or solvate thereof) according to any one of embodiments 3, 6, 7-55, or 71,
   wherein the treatment is administered about once every 4 weeks.
73. The method according to any one of embodiments 1, 4, 7-55, or 71, the compound of Formula A (or a pharmaceutically acceptable salt and/or solvate thereof) for use according to any one of embodiments 2, 5, 7-55, or 71, or the use of the compound of Formula A (or a pharmaceutically acceptable salt and/or solvate thereof) according to any one of embodiments 3, 6, 7-55, or 71,
   wherein the treatment is administered about once every 8 weeks.
74. The method according to any one of embodiments 1, 4, 7-55, or 71, the compound of Formula A (or a pharmaceutically acceptable salt and/or solvate thereof) for use according to any one of embodiments 2, 5, 7-55, or 71, or the use of the compound of Formula A (or a pharmaceutically acceptable salt and/or solvate thereof) according to any one of embodiments 3, 6, 7-55, or 71,
   wherein the treatment is administered about once every 12 weeks.
75. The method according to any one of embodiments 1, 4, 7-55 or 71-74, the compound of Formula A (or a pharmaceutically acceptable salt and/or solvate thereof) for use according to any one of embodiments 2, 5, 7-55 or 71-74, or the use of the compound of Formula A (or a pharmaceutically acceptable salt and/or solvate thereof) according to any one of embodiments 3, 6, 7-55 or 71-74,
   wherein the treatment is administered about regularly for life.
76. A method for treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

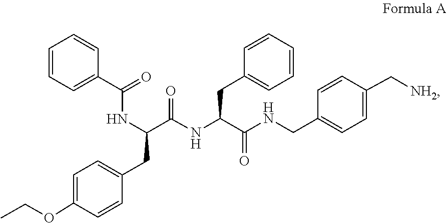 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of DME.
77. The compound of Formula A (or a pharmaceutically acceptable salt and/or solvate thereof) for use in treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

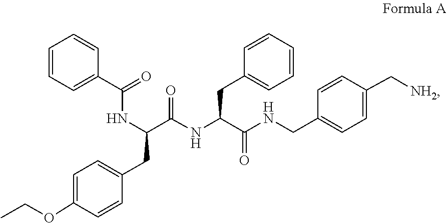 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of DME.
78. The use of the compound of Formula A (or a pharmaceutically acceptable salt and/or solvate thereof) in the manufacture of a medicament for the treatment of diabetic macular edema (DME), comprising: intravitreally administering a pharmaceutical composition, wherein the pharmaceutical composition is a solution comprising the compound of Formula A (or a pharmaceutically acceptable salt and/or solvate thereof), to a patient in need thereof,

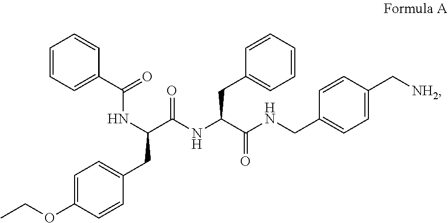 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of DME.
79. A method for treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

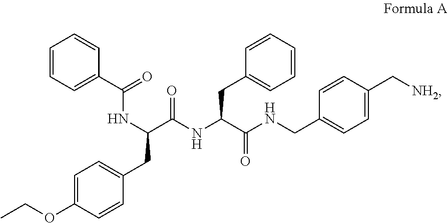 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of impaired visual acuity.
80. The compound of Formula A (or a pharmaceutically acceptable salt and/or solvate thereof) for use in treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

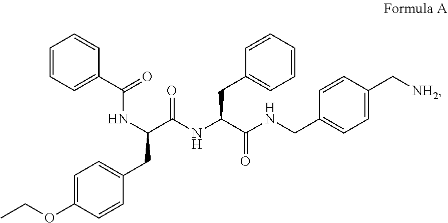 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of impaired visual acuity.
81. The use of the compound of Formula A (or a pharmaceutically acceptable salt and/or solvate thereof) in the manufacture of a medicament for the treatment of impaired visual acuity, comprising: intravitreally administering a pharmaceutical composition, wherein the pharmaceutical composition is a solution comprising the compound of Formula A (or a pharmaceutically acceptable salt and/or solvate thereof), to a patient in need thereof,

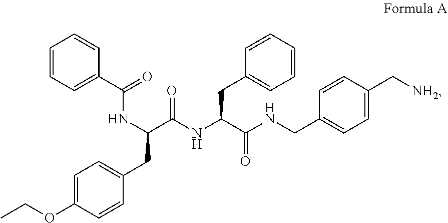 [SMILES: CCOc1ccc(C[C@@H](NC(=O)c2ccccc2)C(=O)N[C@@H](Cc2ccccc2)C(=O)NCc2ccc(CN)cc2)cc1]

wherein the patient is in the early stages of impaired visual acuity.
82. The method according to any one of embodiments 76 or 79, the compound of Formula A (or a pharmaceutically acceptable salt and/or solvate thereof) for use according to any one of embodiments 77 or 80, or the use of the compound of Formula A (or a pharmaceutically acceptable salt and/or solvate thereof) according to any one of embodiments 78 or 81,
   wherein a patient in the early stages of DME or impaired visual acuity is defined by having a baseline visual acuity score (BCVA), prior to administration of the compound of Formula A, of between 56 and 73 letters in at least one eye, measured using the standard Early Treatment Diabetic Retinopathy Study (ETDRS) chart.
83. The method according to any one of embodiments 76 or 79, the compound of Formula A (or a pharmaceutically acceptable salt and/or solvate thereof) for use according to any one of embodiments 77 or 80, or the use of the compound of Formula A (or a pharmaceutically acceptable salt and/or solvate thereof) according to any one of embodiments 78 or 81,
   wherein a patient in the early stages of DME or impaired visual acuity is defined by having a baseline visual acuity score (BCVA), prior to administration of the compound of Formula A, of between 56 and 73 letters in at least one eye, measured using the standard Early Treatment Diabetic Retinopathy Study (ETDRS) chart.
84. The method according to any one of embodiments 76, 79 or 82-83, the compound of Formula A (or a pharmaceutically acceptable salt and/or solvate thereof) for use according to any one of embodiments 77, 80 or 82-83, or the use of the compound of Formula A (or a pharmaceutically acceptable salt and/or solvate thereof) according to any one of embodiments 78, 81 or 82-83,
   wherein the concentration of the compound of Formula A (or a pharmaceutically acceptable salt and/or solvate thereof) when administered is between about 10 μg/mL and about 300 μg/mL based on the concentration of the free base of the compound of Formula A in solution.
85. The method according to any one of embodiments 76, 79 or 82-84, the compound of Formula A (or a pharmaceutically acceptable salt and/or solvate thereof) for use according to any one of embodiments 77, 80 or 82-84, or the use of the compound of Formula A (or a pharmaceutically acceptable salt and/or solvate thereof) according to any one of embodiments 78, 81 or 82-84,
   wherein the concentration of the compound of Formula A (or a pharmaceutically acceptable salt and/or solvate thereof) when administered is between about 10 μg/mL and about 250 μg/mL based on the concentration of the free base of the compound of Formula A in solution.
86. The method according to any one of embodiments 76, 79 or 82-85, the compound of Formula A (or a pharmaceutically acceptable salt and/or solvate thereof) for use according to any one of embodiments 77, 80 or 82-85, or the use of the compound of Formula A (or a pharmaceutically acceptable salt and/or solvate thereof) according to any one of embodiments 78, 81 or 82-85,
   wherein the concentration of the compound of Formula A (or a pharmaceutically acceptable salt and/or solvate thereof) when administered is between about 10 μg/mL and about 200 μg/mL based on the concentration of the free base of the compound of Formula A in solution.
87. The method according to any one of embodiments 76, 79 or 82-86, the compound of Formula A (or a pharmaceutically acceptable salt and/or solvate thereof) for use according to any one of embodiments 77, 80 or 82-86, or the use of the compound of Formula A (or a pharmaceutically acceptable salt and/or solvate thereof) according to any one of embodiments 78, 81 or 82-86,
   wherein the concentration of the compound of Formula A (or a pharmaceutically acceptable salt and/or solvate thereof) when administered is between about 20 μg/mL and about 200 μg/mL based on the concentration of the free base of the compound of Formula A in solution.
88. The method according to any one of embodiments 76, 79 or 82-87, the compound of Formula A (or a pharmaceutically acceptable salt and/or solvate thereof) for use according to any one of embodiments 77, 80 or 82-87, or the use of the compound of Formula A (or a pharmaceutically acceptable salt and/or solvate thereof) according to any one of embodiments 78, 81 or 82-87,
   wherein the concentration of the compound of Formula A (or a pharmaceutically acceptable salt and/or solvate thereof) when administered is between about 20 μg/mL and about 160 μg/mL based on the concentration of the free base of the compound of Formula A in solution.
89. The method according to any one of embodiments 76, 79 or 82-88, the compound of Formula A (or a pharmaceutically acceptable salt and/or solvate thereof) for use according to any one of embodiments 77, 80 or 82-88, or the use of the compound of Formula A (or a pharmaceutically acceptable salt and/or solvate thereof) according to any one of embodiments 78, 81 or 82-88,
   wherein the concentration of the compound of Formula A (or a pharmaceutically acceptable salt and/or solvate thereof) when administered is between about 20 μg/mL and about 120 μg/mL based on the concentration of the free base of the compound of Formula A in solution.
90. The method according to any one of embodiments 76, 79 or 82-89, the compound of Formula A (or a pharmaceutically acceptable salt and/or solvate thereof) for use according to any one of embodiments 77, 80 or 82-89, or the use of the compound of Formula A (or a pharmaceutically acceptable salt and/or solvate thereof) according to any one of embodiments 78, 81 or 82-89,
   wherein the concentration of the compound of formula A (or a pharmaceutically acceptable salt and/or solvate thereof) when administered is between about 30 μg/mL and 100 μg/mL based on the concentration of the free base of the compound of formula A in solution.
91. The method according to any one of embodiments 76, 79 or 82-90, the compound of Formula A (or a pharmaceutically acceptable salt and/or solvate thereof) for use according to any one of embodiments 77, 80 or 82-90, or the use of the compound of Formula A (or a pharmaceutically acceptable salt and/or solvate thereof) according to any one of embodiments 78, 81 or 82-90,
   wherein the concentration of the compound of formula A (or a pharmaceutically acceptable salt and/or solvate thereof) when administered is between about 60 μg/mL and 100 μg/mL based on the concentration of the free base of the compound of formula A in solution.
92. The method according to any one of embodiments 76, 79 or 82-90, the compound of Formula A (or a pharmaceutically acceptable salt and/or solvate thereof) for use according to any one of embodiments 77, 80 or 82-90, or the use of the compound of Formula A (or a pharmaceutically acceptable salt and/or solvate thereof) according to any one of embodiments 78, 81 or 82-90,
   wherein the concentration of the compound of Formula A (or a pharmaceutically acceptable salt and/or solvate thereof) when administered is about 30 μg/mL based on the concentration of the free base of the compound of Formula A in solution.
93. The method according to any one of embodiments 76, 79 or 82-91, the compound of Formula A (or a pharmaceutically acceptable salt and/or solvate thereof) for use according to any one of embodiments 77, 80 or 82-91, or the use of the compound of Formula A (or a pharmaceutically acceptable salt and/or solvate thereof) according to any one of embodiments 78, 81 or 82-91,
   wherein the concentration of the compound of Formula A (or a pharmaceutically acceptable salt and/or solvate thereof) when administered is about 60 μg/mL based on the concentration of the free base of the compound of Formula A in solution.
94. The method according to any one of embodiments 76, 79 or 82-91, the compound of Formula A (or a pharmaceutically acceptable salt and/or solvate thereof) for use according to any one of embodiments 77, 80 or 82-91, or the use of the compound of Formula A (or a pharmaceutically acceptable salt and/or solvate thereof) according to any one of embodiments 78, 81 or 82-91,
   wherein the concentration of the compound of Formula A (or a pharmaceutically acceptable salt and/or solvate thereof) when administered is about 100 μg/mL based on the concentration of the free base of the compound of Formula A in solution.
95. The method according to any one of embodiments 76, 79 or 82-89, the compound of Formula A (or a pharmaceutically acceptable salt and/or solvate thereof) for use according to any one of embodiments 77, 80 or 82-89, or the use of the compound of Formula A (or a pharmaceutically acceptable salt and/or solvate thereof) according to any one of embodiments 78, 81 or 82-89,
   wherein the concentration of the compound of Formula A (or a pharmaceutically acceptable salt and/or solvate thereof) when administered is about 120 μg/mL based on the concentration of the free base of the compound of Formula A in solution.
96. The method according to any one of embodiments 76, 79 or 82-87, the compound of Formula A (or a pharmaceutically acceptable salt and/or solvate thereof) for use according to any one of embodiments 77, 80 or 82-87, or the use of the compound of Formula A (or a pharmaceutically acceptable salt and/or solvate thereof) according to any one of embodiments 78, 81 or 82-87,
   wherein the concentration of the compound of Formula A (or a pharmaceutically acceptable salt and/or solvate thereof) when administered is about 200 μg/mL based on the concentration of the free base of the compound of Formula A in solution.
97. The method according to any one of embodiments 76, 79 or 82-96, the compound of Formula A (or a pharmaceutically acceptable salt and/or solvate thereof) for use according to any one of embodiments 77, 80 or 82-96, or the use of the compound of Formula A (or a pharmaceutically acceptable salt and/or solvate thereof) according to any one of embodiments 78, 81 or 82-96,
   wherein about 10 μL to about 100 μL of the solution is administered per intravitreal administration.
98. The method according to any one of embodiments 76, 79 or 82-97, the compound of Formula A (or a pharmaceutically acceptable salt and/or solvate thereof) for use according to any one of embodiments 77, 80 or 82-97, or the use of the compound of Formula A (or a pharmaceutically acceptable salt and/or solvate thereof) according to any one of embodiments 78, 81 or 82-97,
   wherein about 25 uL to about 100 uL of the solution is administered per intravitreal administration.
99. The method according to any one of embodiments 76, 79 or 82-98, the compound of Formula A (or a pharmaceutically acceptable salt and/or solvate thereof) for use according to any one of embodiments 77, 80 or 82-98, or the use of the compound of Formula A (or a pharmaceutically acceptable salt and/or solvate thereof) according to any one of embodiments 78, 81 or 82-98,
   wherein about 50 uL to about 100 uL of the solution is administered per intravitreal administration.
100. The method according to any one of embodiments 76, 79 or 82-99, the compound of Formula A (or a pharmaceutically acceptable salt and/or solvate thereof) for use according to any one of embodiments 77, 80 or 82-99, or the use of the compound of Formula A (or a pharmaceutically acceptable salt and/or solvate thereof) according to any one of embodiments 78, 81 or 82-99,
   wherein about 50 μL to about 60 μL of the solution is administered per intravitreal administration.
101. The method according to any one of embodiments 76, 79 or 82-99, the compound of Formula A (or a pharmaceutically acceptable salt and/or solvate thereof) for use according to any one of embodiments 77, 80 or 82-99, or the use of the compound of Formula A (or a pharmaceutically acceptable salt and/or solvate thereof) according to any one of embodiments 78, 81 or 82-99,
   wherein about 60 μL to about 70 μL of the solution is administered per intravitreal administration.
102. The method according to any one of embodiments 76, 79 or 82-99, the compound of Formula A (or a pharmaceutically acceptable salt and/or solvate thereof) for use according to any one of embodiments 77, 80 or 82-99, or the use of the compound of Formula A (or a pharmaceutically acceptable salt and/or solvate thereof) according to any one of embodiments 78, 81 or 82-99,
   wherein about 70 μL to about 80 μL of the solution is administered per intravitreal administration.
103. The method according to any one of embodiments 76, 79 or 82-99, the compound of Formula A (or a pharmaceutically acceptable salt and/or solvate thereof) for use according to any one of embodiments 77, 80 or 82-99, or the use of the compound of Formula A (or a pharmaceutically acceptable salt and/or solvate thereof) according to any one of embodiments 78, 81 or 82-99,
   wherein about 80 μL to about 90 μL of the solution is administered per intravitreal administration.
104. The method according to any one of embodiments 76, 79 or 82-99, the compound of Formula A (or a pharmaceutically acceptable salt and/or solvate thereof) for use according to any one of embodiments 77, 80 or 82-99, or the use of the compound of Formula A (or a pharmaceutically acceptable salt and/or solvate thereof) according to any one of embodiments 78, 81 or 82-99,
   wherein about 90 μL to about 100 μL of the solution is administered per intravitreal administration.
105. The method according to any one of embodiments 76, 79 or 82-100, the compound of Formula A (or a pharmaceutically acceptable salt and/or solvate thereof) for use according to any one of embodiments 77, 80 or 82-100, or the use of the compound of Formula A (or a pharmaceutically acceptable salt and/or solvate thereof) according to any one of embodiments 78, 81 or 82-100,
   wherein about 50 μL of the solution is administered per intravitreal administration.
106. The method according to any one of embodiments 76, 79 or 82-101, the compound of Formula A (or a pharmaceutically acceptable salt and/or solvate thereof) for use according to any one of embodiments 77, 80 or 82-101, or the use of the compound of Formula A (or a pharmaceutically acceptable salt and/or solvate thereof) according to any one of embodiments 78, 81 or 82-101,
   wherein about 60 μL of the solution is administered per intravitreal administration.
107. The method according to any one of embodiments 76, 79 or 82-99 or 101-102, the compound of Formula A (or a pharmaceutically acceptable salt and/or solvate thereof) for use according to any one of embodiments 77, 80 or 82-99 or 101-102, or the use of the compound of Formula A (or a pharmaceutically acceptable salt and/or solvate thereof) according to any one of embodiments 78, 81 or 82-99 or 101-102,
   wherein about 70 μL of the solution is administered per intravitreal administration.
108. The method according to any one of embodiments 76, 79 or 82-99 or 102-103, the compound of Formula A (or a pharmaceutically acceptable salt and/or solvate thereof) for use according to any one of embodiments 77, 80 or 82-99 or 102-103, or the use of the compound of Formula A (or a pharmaceutically acceptable salt and/or solvate thereof) according to any one of embodiments 78, 81 or 82-99 or 102-103,
   wherein about 80 μL of the solution is administered per intravitreal administration.
109. The method according to any one of embodiments 76, 79 or 82-99 or 103104, the compound of Formula A (or a pharmaceutically acceptable salt and/or solvate thereof) for use according to any one of embodiments 77, 80 or 82-99 or 103-104, or the use of the compound of Formula A (or a pharmaceutically acceptable salt and/or solvate thereof) according to any one of embodiments 78, 81 or 82-99 or 103-104,
   wherein about 90 μL of the solution is administered per intravitreal administration.
110. The method according to any one of embodiments 76, 79 or 82-99 or 104, the compound of Formula A (or a pharmaceutically acceptable salt and/or solvate thereof) for use according to any one of embodiments 77, 80 or 82-99 or 104, or the use of the compound of Formula A (or a pharmaceutically acceptable salt and/or solvate thereof) according to any one of embodiments 78, 81 or 82-99 or 104,
   wherein about 100 μL of the solution is administered per intravitreal administration.
111. The method according to any one of embodiments 76, 79 or 82-110, the compound of Formula A (or a pharmaceutically acceptable salt and/or solvate thereof) for use according to any one of embodiments 77, 80 or 82-110, or the use of the compound of Formula A (or a pharmaceutically acceptable salt and/or solvate thereof) according to any one of embodiments 78, 81 or 82-110,
   wherein the solution further comprises at least one non-ionic tonicity agent.
112. The method according to embodiment 111, the compound of Formula A (or a pharmaceutically acceptable salt and/or solvate thereof) for use according to embodiment 111, or the use of the compound of Formula A (or a pharmaceutically acceptable salt and/or solvate thereof) according to embodiment 111,
   wherein the at least one non-ionic tonicity agent is trehalose.
113. The method according to embodiment 112, the compound of Formula A (or a pharmaceutically acceptable salt and/or solvate thereof) for use according to embodiment 112, or the use of the compound of Formula A (or a pharmaceutically acceptable salt and/or solvate thereof) according to embodiment 112,
   wherein the trehalose is provided as trehalose dihydrate.
114. The method according to any one of embodiments 76, 79 or 82-113, the compound of Formula A (or a pharmaceutically acceptable salt and/or solvate thereof) for use according to any one of embodiments 77, 80 or 82-113, or the use of the compound of Formula A (or a pharmaceutically acceptable salt and/or solvate thereof) according to any one of embodiments 78, 81 or 82-113,
   wherein the solution further comprises histidine.
115. The method according to any one of embodiments 76, 79 or 82-114, the compound of Formula A (or a pharmaceutically acceptable salt and/or solvate thereof) for use according to any one of embodiments 77, 80 or 82-114, or the use of the compound of Formula A (or a pharmaceutically acceptable salt and/or solvate thereof) according to any one of embodiments 78, 81 or 82-114,
   wherein the pharmaceutical composition comprises an aqueous solution of the compound of formula A (or a pharmaceutically acceptable salt and/or solvate thereof), histidine and trehalose dihydrate.
116. The method according to any one of embodiments 76, 79 or 82-115, the compound of Formula A (or a pharmaceutically acceptable salt and/or solvate thereof) for use according to any one of embodiments 77, 80 or 82-115, or the use of the compound of Formula A (or a pharmaceutically acceptable salt and/or solvate thereof) according to any one of embodiments 78, 81 or 82-115,
   wherein the pharmaceutical composition has a pH from about 2 to about 10, preferably from about 5 to about 7.5, preferably from about 5.3 to about 6, and preferably from about 5.4 to about 5.8.
117. The method according to any one of embodiments 76, 79 or 82-116, the compound of Formula A (or a pharmaceutically acceptable salt and/or solvate thereof) for use according to any one of embodiments 77, 80 or 82-116, or the use of the compound of Formula A (or a pharmaceutically acceptable salt and/or solvate thereof) according to any one of embodiments 78, 81 or 82-116,
   wherein the pharmaceutical composition has a pH of about 5.5.
118. The method according to any one of embodiments 76, 79 or 82-117, the compound of Formula A (or a pharmaceutically acceptable salt and/or solvate thereof) for use according to any one of embodiments 77, 80 or 82-117, or the use of the compound of Formula A (or a pharmaceutically acceptable salt and/or solvate thereof) according to any one of embodiments 78, 81 or 82-117,
   wherein the treatment is a monotherapy for DME or impaired visual acuity.
119. The method according to any one of embodiments 76, 79 or 82-118, the compound of Formula A (or a pharmaceutically acceptable salt and/or solvate thereof) for use according to any one of embodiments 77, 80 or 82-118, or the use of the compound of Formula A (or a pharmaceutically acceptable salt and/or solvate thereof) according to any one of embodiments 78, 81 or 82-118, wherein the patient has previously had anti-VEGF (vascular endothelial growth factor) treatment.
120. The method according to embodiment 119, the compound of Formula A (or a pharmaceutically acceptable salt and/or solvate thereof) for use according to embodiment 119, or the use of the compound of Formula A (or a pharmaceutically acceptable salt and/or solvate thereof) according to embodiments 119,
   wherein the anti-VEGF treatment is selected from aflibercept (Eylea®), bevacizumab, ranibizumab, and pegaptanib.
121. The method according to any one of embodiments 119-120, the compound of Formula A (or a pharmaceutically acceptable salt and/or solvate thereof) for use according to any one of embodiments 119-120, or the use of the compound of Formula A (or a pharmaceutically acceptable salt and/or solvate thereof) according to any one of embodiments 119-120,
   wherein the anti-VEGF is aflibercept (Eylea®).
122. The method according to any one of embodiments 119-121, the compound of Formula A (or a pharmaceutically acceptable salt and/or solvate thereof) for use according to any one of embodiments 119-121, or the use of the compound of Formula A (or a pharmaceutically acceptable salt and/or solvate thereof) according to any one of embodiments 119-121,
   wherein the patient received anti-VEGF treatment for no more than 36 months before commencing treatment with the compound of Formula A (or a pharmaceutically acceptable salt and/or solvate thereof).
123. The method according to any one of embodiments 119-122, the compound of Formula A (or a pharmaceutically acceptable salt and/or solvate thereof) for use according to any one of embodiments 119-122, or the use of the compound of Formula A (or a pharmaceutically acceptable salt and/or solvate thereof) according to any one of embodiments 119-122,
   wherein the patient received anti-VEGF treatment no less than 8 weeks before commencing treatment with the compound of formula A (or a pharmaceutically acceptable salt and/or solvate thereof).
124. The method according to any one of embodiments 76, 79 or 82-123, the compound of Formula A (or a pharmaceutically acceptable salt and/or solvate thereof) for use according to any one of embodiments 77, 80 or 82-123, or the use of the compound of Formula A (or a pharmaceutically acceptable salt and/or solvate thereof) according to any one of embodiments 78, 81 or 82-123,
   wherein the patient does not receive anti-VEGF treatment concurrent to administration of the compound of Formula A.
125. The method according to any one of embodiments 76, 79 or 82-124, the compound of Formula A (or a pharmaceutically acceptable salt and/or solvate thereof) for use according to any one of embodiments 77, 80 or 82-124, or the use of the compound of Formula A (or a pharmaceutically acceptable salt and/or solvate thereof) according to any one of embodiments 78, 81 or 82-124,
   wherein the treatment is administered over a time period of at least about 12 weeks.
126. The method according to any one of embodiments 76, 79 or 82-125, the compound of Formula A (or a pharmaceutically acceptable salt and/or solvate thereof) for use according to any one of embodiments 77, 80 or 82-125, or the use of the compound of Formula A (or a pharmaceutically acceptable salt and/or solvate thereof) according to any one of embodiments 78, 81 or 82-125,
   wherein the treatment is administered at a first dosing frequency over a first time period, followed by a second dosing frequency over a second time period, wherein the second dosing frequency is lower than the first dosing frequency.
127. The method according to embodiments 126, the compound of Formula A (or a pharmaceutically acceptable salt and/or solvate thereof) for use according to embodiments 126, or the use of the compound of Formula A (or a pharmaceutically acceptable salt and/or solvate thereof) according to embodiments 126,
   wherein the first time period is greater than about 8 weeks.
128. The method according to any one of embodiments 126-127, the compound of Formula A (or a pharmaceutically acceptable salt and/or solvate thereof) for use according to any one of embodiments 126-127, or the use of the compound of Formula A (or a pharmaceutically acceptable salt and/or solvate thereof) according to any one of embodiments 126-127,
   wherein the first time period is greater than about 12 weeks.
129. The method according to any one of embodiments 126-127, the compound of Formula A (or a pharmaceutically acceptable salt and/or solvate thereof) for use according to any one of embodiments 126-127, or the use of the compound of Formula A (or a pharmaceutically acceptable salt and/or solvate thereof) according to any one of embodiments 126-127,
   wherein the first time period is between about 10 weeks and about 12 weeks.
130. The method according to any one of embodiments 126-129, the compound of Formula A (or a pharmaceutically acceptable salt and/or solvate thereof) for use according to any one of embodiments 126-129, or the use of the compound of Formula A (or a pharmaceutically acceptable salt and/or solvate thereof) according to any one of embodiments 126-129,
   wherein the first time period is about 12 weeks.
131. The method according to any one of embodiments 126-130, the compound of Formula A (or a pharmaceutically acceptable salt and/or solvate thereof) for use according to any one of embodiments 126-130, or the use of the compound of Formula A (or a pharmaceutically acceptable salt and/or solvate thereof) according to any one of embodiments 126-130,
   wherein the first dosing frequency is between about once every three weeks and about once every five weeks.
132. The method according to embodiment 131, the compound of Formula A (or a pharmaceutically acceptable salt and/or solvate thereof) for use according to embodiment 131, or the use of the compound of Formula A (or a pharmaceutically acceptable salt and/or solvate thereof) according embodiment 131,
   wherein the first dosing frequency is about once every four weeks.
133. The method according to any one of embodiments 126-132, the compound of Formula A (or a pharmaceutically acceptable salt and/or solvate thereof) for use according to any one of embodiments 126-132, or the use of the compound of Formula A (or a pharmaceutically acceptable salt and/or solvate thereof) according to any one of embodiments 126-132,
   wherein the second time period is greater than about 8 weeks.
134. The method according to any one of embodiments 126-133, the compound of Formula A (or a pharmaceutically acceptable salt and/or solvate thereof) for use according to any one of embodiments 126-133, or the use of the compound of Formula A (or a pharmaceutically acceptable salt and/or solvate thereof) according to any one of embodiments 126-133,
   wherein the second time period is greater than about 12 weeks.
135. The method according to any one of embodiments 126-134, the compound of Formula A (or a pharmaceutically acceptable salt and/or solvate thereof) for use according to any one of embodiments 126-134, or the use of the compound of Formula A (or a pharmaceutically acceptable salt and/or solvate thereof) according to any one of embodiments 126-134,
   wherein the second time period is greater than about 16 weeks.
136. The method according to any one of embodiments 126-133, the compound of Formula A (or a pharmaceutically acceptable salt and/or solvate thereof) for use according to any one of embodiments 126-133, or the use of the compound of Formula A (or a pharmaceutically acceptable salt and/or solvate thereof) according to any one of embodiments 126-133,
   wherein the second time period is between about 8 weeks and about 12 weeks.
137. The method according to any one of embodiments 126-133, the compound of Formula A (or a pharmaceutically acceptable salt and/or solvate thereof) for use according to any one of embodiments 126-133, or the use of the compound of Formula A (or a pharmaceutically acceptable salt and/or solvate thereof) according to any one of embodiments 126-133,
   wherein the second time period is about 12 weeks.
138. The method according to any one of embodiments 126-137, the compound of Formula A (or a pharmaceutically acceptable salt and/or solvate thereof) for use according to any one of embodiments 126-137, or the use of the compound of Formula A (or a pharmaceutically acceptable salt and/or solvate thereof) according to any one of embodiments 126-137,
   wherein the second dosing frequency is lower than about once every six weeks.
139. The method according to any one of embodiments 126-138, the compound of Formula A (or a pharmaceutically acceptable salt and/or solvate thereof) for use according to any one of embodiments 126-138, or the use of the compound of Formula A (or a pharmaceutically acceptable salt and/or solvate thereof) according to any one of embodiments 126-138,
   wherein the second dosing frequency is lower than about once every eight weeks.
140. The method according to any one of embodiments 126-139, the compound of Formula A (or a pharmaceutically acceptable salt and/or solvate thereof) for use according to any one of embodiments 126-139, or the use of the compound of Formula A (or a pharmaceutically acceptable salt and/or solvate thereof) according to any one of embodiments 126-139,
   wherein the second dosing frequency is lower than about once every twelve weeks.
141. The method according to any one of embodiments 76, 79 or 82-125, the compound of Formula A (or a pharmaceutically acceptable salt and/or solvate thereof) for use according to any one of embodiments 77, 80 or 82-125, or the use of the compound of Formula A (or a pharmaceutically acceptable salt and/or solvate thereof) according to any one of embodiments 78, 81 or 82-125,
   wherein the treatment is administered between about once every 4 weeks and once every 12 weeks.
142. The method according to any one of embodiments 76, 79 or 82-125 or 141, the compound of Formula A (or a pharmaceutically acceptable salt and/or solvate thereof) for use according to any one of embodiments 77, 80 or 82-125 or 141, or the use of the compound of Formula A (or a pharmaceutically acceptable salt and/or solvate thereof) according to any one of embodiments 78, 81 or 82-125 or 141,
   wherein the treatment is administered about once every 4 weeks.
143. The method according to any one of embodiments 76, 79 or 82-125 or 141, the compound of Formula A (or a pharmaceutically acceptable salt and/or solvate thereof) for use according to any one of embodiments 77, 80 or 82-125 or 141, or the use of the compound of Formula A (or a pharmaceutically acceptable salt and/or solvate thereof) according to any one of embodiments 78, 81 or 82-125 or 141,
   wherein the treatment is administered about once every 8 weeks.
144. The method according to any one of embodiments 76, 79 or 82-125 or 141, the compound of Formula A (or a pharmaceutically acceptable salt and/or solvate thereof) for use according to any one of embodiments 77, 80 or 82-125 or 141, or the use of the compound of Formula A (or a pharmaceutically acceptable salt and/or solvate thereof) according to any one of embodiments 78, 81 or 82-125 or 141,
   wherein the treatment is administered about once every 12 weeks.
145. The method according to any one of embodiments 76, 79 or 82-125 or 141-144, the compound of Formula A (or a pharmaceutically acceptable salt and/or solvate thereof) for use according to any one of embodiments 77, 80 or 82-125 or 141-144, or the use of the compound of Formula A (or a pharmaceutically acceptable salt and/or solvate thereof) according to any one of embodiments 78, 81 or 82-125 or 141-144,
   wherein the treatment is administered regularly for life.
146. A method for treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

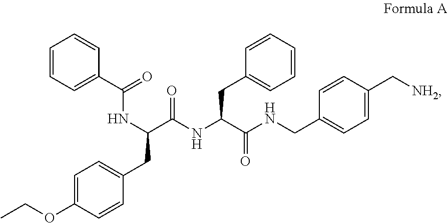 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.
147. The compound of Formula A (or a pharmaceutically acceptable salt and/or solvate thereof) for use in treating diabetic macular edema (DME) comprising: intravitreally administering a pharmaceutical composition, wherein the pharmaceutical composition is a solution comprising the compound of Formula A (or a pharmaceutically acceptable salt and/or solvate thereof), to a patient in need thereof,

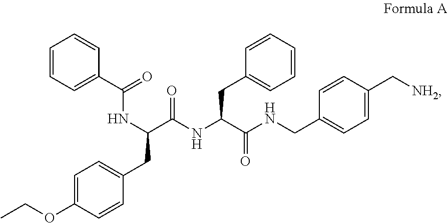 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.
148. The use of the compound of Formula A (or a pharmaceutically acceptable salt and/or solvate thereof) in the manufacture of a medicament for the treatment of diabetic macular edema (DME), comprising: intravitreally administering a pharmaceutical composition, wherein the pharmaceutical composition is a solution comprising the compound of Formula A (or a pharmaceutically acceptable salt and/or solvate thereof), to a patient in need thereof,

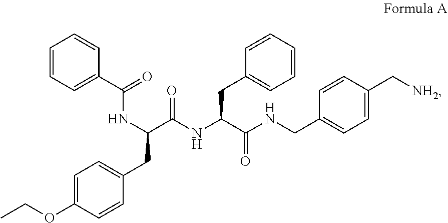 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.
149. A method for treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

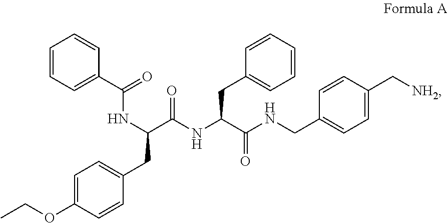 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.
150. The compound of Formula A (or a pharmaceutically acceptable salt and/or solvate thereof) for use in treating impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof,

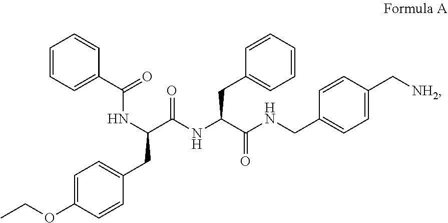 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.
151. The use of the compound of Formula A (or a pharmaceutically acceptable salt and/or solvate thereof) in the manufacture of a medicament for the treatment of impaired visual acuity, comprising: intravitreally administering a pharmaceutical composition, wherein the pharmaceutical composition is a solution comprising the compound of Formula A (or a pharmaceutically acceptable salt and/or solvate thereof), to a patient in need thereof,

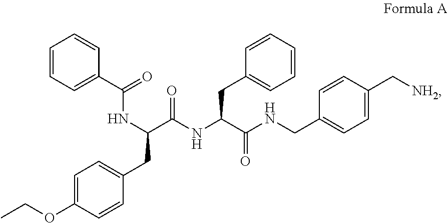 [SMILES: CCOc1ccc(C[C@@H](NC(=O)c2ccccc2)C(=O)N[C@@H](Cc2ccccc2)C(=O)NCc2ccc(CN)cc2)cc1]

wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.
152. The method according to any one of embodiments 146 or 149, the compound of Formula A (or a pharmaceutically acceptable salt and/or solvate thereof) for use according to any one of embodiments 147 or 150, or the use of the compound of Formula A (or a pharmaceutically acceptable salt and/or solvate thereof) according to any one of embodiments 148 or 151,
   wherein the treatment is administered at a first dosing frequency over a first time period wherein the concentration of the compound of formula A (or a pharmaceutically acceptable salt and/or solvate thereof) when administered in the first time period is between about 60 μg/mL and about 300 μg/mL based on the concentration of the free base of the compound of formula A in solution.
153. The method according to any one of embodiments 146, 149 or 152, the compound of Formula A (or a pharmaceutically acceptable salt and/or solvate thereof) for use according to any one of embodiments 147, 150 or 152, or the use of the compound of Formula A (or a pharmaceutically acceptable salt and/or solvate thereof) according to any one of embodiments 148, 151 or 152,
   wherein the treatment is administered at a first dosing frequency over a first time period wherein the concentration of the compound of formula A (or a pharmaceutically acceptable salt and/or solvate thereof) when administered in the first time period is between about 60 μg/mL and about 200 μg/mL based on the concentration of the free base of the compound of formula A in solution.
154. The method according to any one of embodiments 146, 149 or 152-153, the compound of Formula A (or a pharmaceutically acceptable salt and/or solvate thereof) for use according to any one of embodiments 147, 150 or 152-153, or the use of the compound of Formula A (or a pharmaceutically acceptable salt and/or solvate thereof) according to any one of embodiments 148, 151 or 152-153,
   wherein the treatment is administered at a first dosing frequency over a first time period wherein the concentration of the compound of formula A (or a pharmaceutically acceptable salt and/or solvate thereof) when administered in the first time period is between about 60 μg/mL and about 100 μg/mL based on the concentration of the free base of the compound of formula A in solution.
155. The method according to any one of embodiments 146, 149 or 152-154, the compound of Formula A (or a pharmaceutically acceptable salt and/or solvate thereof) for use according to any one of embodiments 147, 150 or 152-154, or the use of the compound of Formula A (or a pharmaceutically acceptable salt and/or solvate thereof) according to any one of embodiments 148, 151 or 152-154,
   wherein the treatment is administered at a first dosing frequency over a first time period wherein the concentration of the compound of formula A (or a pharmaceutically acceptable salt and/or solvate thereof) when administered in the first time period is about 60 μg/mL based on the concentration of the free base of the compound of Formula A in solution.
156. The method according to any one of embodiments 146, 149 or 152-154, the compound of Formula A (or a pharmaceutically acceptable salt and/or solvate thereof) for use according to any one of embodiments 147, 150 or 152-154, or the use of the compound of Formula A (or a pharmaceutically acceptable salt and/or solvate thereof) according to any one of embodiments 148,151 or 152-154,
   wherein the treatment is administered at a first dosing frequency over a first time period wherein the concentration of the compound of formula A (or a pharmaceutically acceptable salt and/or solvate thereof) when administered in the first time period is about 100 μg/mL based on the concentration of the free base of the compound of Formula A in solution.
157. The method according to any one of embodiments 146, 149 or 152-153, the compound of Formula A (or a pharmaceutically acceptable salt and/or solvate thereof) for use according to any one of embodiments 147, 150 or 152-153, or the use of the compound of Formula A (or a pharmaceutically acceptable salt and/or solvate thereof) according to any one of embodiments 148, 151 or 152-153,
   wherein the treatment is administered at a first dosing frequency over a first time period wherein the concentration of the compound of formula A (or a pharmaceutically acceptable salt and/or solvate thereof) when administered in the first time period is about 120 μg/mL based on the concentration of the free base of the compound of Formula A in solution.
158. The method according to any one of embodiments 146, 149 or 152-153, the compound of Formula A (or a pharmaceutically acceptable salt and/or solvate thereof) for use according to any one of embodiments 147, 150 or 152-153, or the use of the compound of Formula A (or a pharmaceutically acceptable salt and/or solvate thereof) according to any one of embodiments 148, 151 or 152-153,
   wherein the treatment is administered at a first dosing frequency over a first time period wherein the concentration of the compound of formula A (or a pharmaceutically acceptable salt and/or solvate thereof) when administered in the first time period is about 200 μg/mL based on the concentration of the free base of the compound of Formula A in solution.
159. The method according to any one of embodiments 146, 149 or 152-158, the compound of Formula A (or a pharmaceutically acceptable salt and/or solvate thereof) for use according to any one of embodiments 147, 150 or 152-158, or the use of the compound of Formula A (or a pharmaceutically acceptable salt and/or solvate thereof) according to any one of embodiments 148, 151 or 152-158,
   wherein the concentration of the compound of Formula A (or a pharmaceutically acceptable salt and/or solvate thereof) when administered in the second time period is between about 10 μg/mL and about 300 μg/mL based on the concentration of the free base of the compound of Formula A in solution.
160. The method according to any one of embodiments 146, 149 or 152-159, the compound of Formula A (or a pharmaceutically acceptable salt and/or solvate thereof) for use according to any one of embodiments 147, 150 or 152-159, or the use of the compound of Formula A (or a pharmaceutically acceptable salt and/or solvate thereof) according to any one of embodiments 148, 151 or 152-159,
   wherein the concentration of the compound of Formula A (or a pharmaceutically acceptable salt and/or solvate thereof) when administered in the second time period is between about 10 μg/mL and about 250 μg/mL based on the concentration of the free base of the compound of Formula A in solution.
161. The method according to any one of embodiments 146, 149 or 152-160, the compound of Formula A (or a pharmaceutically acceptable salt and/or solvate thereof) for use according to any one of embodiments 147, 150 or 152-160, or the use of the compound of Formula A (or a pharmaceutically acceptable salt and/or solvate thereof) according to any one of embodiments 148, 151 or 152-160,
   wherein the concentration of the compound of Formula A (or a pharmaceutically acceptable salt and/or solvate thereof) when administered in the second time period is between about 10 μg/mL and about 200 μg/mL based on the concentration of the free base of the compound of Formula A in solution.
162. The method according to any one of embodiments 146, 149 or 152-161, the compound of Formula A (or a pharmaceutically acceptable salt and/or solvate thereof) for use according to any one of embodiments 147, 150 or 152-161, or the use of the compound of Formula A (or a pharmaceutically acceptable salt and/or solvate thereof) according to any one of embodiments 148, 151 or 152-161,
   wherein the concentration of the compound of Formula A (or a pharmaceutically acceptable salt and/or solvate thereof) when administered in the second time period is between about 20 μg/mL and about 200 μg/mL based on the concentration of the free base of the compound of Formula A in solution.
163. The method according to any one of embodiments 146, 149 or 152-162, the compound of Formula A (or a pharmaceutically acceptable salt and/or solvate thereof) for use according to any one of embodiments 147, 150 or 152-162, or the use of the compound of Formula A (or a pharmaceutically acceptable salt and/or solvate thereof) according to any one of embodiments 148, 151 or 152-162,
   wherein the concentration of the compound of Formula A (or a pharmaceutically acceptable salt and/or solvate thereof) when administered in the second time period is between about 20 μg/mL and about 160 μg/mL based on the concentration of the free base of the compound of Formula A in solution.
164. The method according to any one of embodiments 146, 149 or 152-163, the compound of Formula A (or a pharmaceutically acceptable salt and/or solvate thereof) for use according to any one of embodiments 147, 150 or 152-163, or the use of the compound of Formula A (or a pharmaceutically acceptable salt and/or solvate thereof) according to any one of embodiments 148, 151 or 152-163,
   wherein the concentration of the compound of Formula A (or a pharmaceutically acceptable salt and/or solvate thereof) when administered in the second time period is between about 20 μg/mL and about 120 μg/mL based on the concentration of the free base of the compound of Formula A in solution.
165. The method according to any one of embodiments 146, 149 or 152-164, the compound of Formula A (or a pharmaceutically acceptable salt and/or solvate thereof) for use according to any one of embodiments 147, 150 or 152-164, or the use of the compound of Formula A (or a pharmaceutically acceptable salt and/or solvate thereof) according to any one of embodiments 148, 151 or 152-164,
   wherein the concentration of the compound of formula A (or a pharmaceutically acceptable salt and/or solvate thereof) when administered in the second time period is between about 30 μg/mL and 100 μg/mL based on the concentration of the free base of the compound of formula A in solution.
166. The method according to any one of embodiments 146, 149 or 152-165 the compound of Formula A (or a pharmaceutically acceptable salt and/or solvate thereof) for use according to any one of embodiments 147, 150 or 152-165, or the use of the compound of Formula A (or a pharmaceutically acceptable salt and/or solvate thereof) according to any one of embodiments 148, 151 or 152-165,
   wherein the concentration of the compound of formula A (or a pharmaceutically acceptable salt and/or solvate thereof) when administered in the second time period is between about 60 μg/mL and 100 μg/mL based on the concentration of the free base of the compound of formula A in solution.
167. The method according to any one of embodiments 146, 149 or 152-165, the compound of Formula A (or a pharmaceutically acceptable salt and/or solvate thereof) for use according to any one of embodiments 147, 150 or 152-165, or the use of the compound of Formula A (or a pharmaceutically acceptable salt and/or solvate thereof) according to any one of embodiments 148, 151 or 152-165,
   wherein the concentration of the compound of Formula A (or a pharmaceutically acceptable salt and/or solvate thereof) when administered in the second time period is about 30 μg/mL based on the concentration of the free base of the compound of Formula A in solution.
168. The method according to any one of embodiments 146, 149 or 152-166, the compound of Formula A (or a pharmaceutically acceptable salt and/or solvate thereof) for use according to any one of embodiments 147, 150 or 152-166, or the use of the compound of Formula A (or a pharmaceutically acceptable salt and/or solvate thereof) according to any one of embodiments 148, 151 or 152-166,
   wherein the concentration of the compound of Formula A (or a pharmaceutically acceptable salt and/or solvate thereof) when administered in the second time period is about 60 μg/mL based on the concentration of the free base of the compound of Formula A in solution.
169. The method according to any one of embodiments 146, 149 or 152-166, the compound of Formula A (or a pharmaceutically acceptable salt and/or solvate thereof) for use according to any one of embodiments 147, 150 or 152-166, or the use of the compound of Formula A (or a pharmaceutically acceptable salt and/or solvate thereof) according to any one of embodiments 148, 151 or 152-166,
   wherein the concentration of the compound of Formula A (or a pharmaceutically acceptable salt and/or solvate thereof) when administered in the second time period is about 100 μg/mL based on the concentration of the free base of the compound of Formula A in solution.
170. The method according to any one of embodiments 146, 149 or 152-164, the compound of Formula A (or a pharmaceutically acceptable salt and/or solvate thereof) for use according to any one of embodiments 147, 150 or 152-164, or the use of the compound of Formula A (or a pharmaceutically acceptable salt and/or solvate thereof) according to any one of embodiments 148, 151 or 152-164,
   wherein the concentration of the compound of Formula A (or a pharmaceutically acceptable salt and/or solvate thereof) when administered in the second time period is about 120 μg/mL based on the concentration of the free base of the compound of Formula A in solution.
171. The method according to any one of embodiments 146, 149 or 152-162, the compound of Formula A (or a pharmaceutically acceptable salt and/or solvate thereof) for use according to any one of embodiments 147, 150 or 152-162, or the use of the compound of Formula A (or a pharmaceutically acceptable salt and/or solvate thereof) according to any one of embodiments 148, 151 or 152-162,
   wherein the concentration of the compound of Formula A (or a pharmaceutically acceptable salt and/or solvate thereof) when administered in the second time period is about 200 μg/mL based on the concentration of the free base of the compound of Formula A in solution.
172. The method according to any one of embodiments 146, 149 or 152-171, the compound of Formula A (or a pharmaceutically acceptable salt and/or solvate thereof) for use according to any one of embodiments 147, 150 or 152-171, or the use of the compound of Formula A (or a pharmaceutically acceptable salt and/or solvate thereof) according to any one of embodiments 148, 151 or 152-171,
   wherein about 100 μL to about 100 μL of the solution is administered per intravitreal administration.
173. The method according to any one of embodiments 146, 149 or 152-172, the compound of Formula A (or a pharmaceutically acceptable salt and/or solvate thereof) for use according to any one of embodiments 147, 150 or 152-172, or the use of the compound of Formula A (or a pharmaceutically acceptable salt and/or solvate thereof) according to any one of embodiments 148, 151 or 152-172,
   wherein about 25 μL to about 100 μL of the solution is administered per intravitreal administration.
174. The method according to any one of embodiments 146, 149 or 152-173, the compound of Formula A (or a pharmaceutically acceptable salt and/or solvate thereof) for use according to any one of embodiments 147, 150 or 152-173, or the use of the compound of Formula A (or a pharmaceutically acceptable salt and/or solvate thereof) according to any one of embodiments 148, 151 or 152-173,
   wherein about 50 μL to about 100 μL of the solution is administered per intravitreal administration.
175. The method according to any one of embodiments 146, 149 or 152-174, the compound of Formula A (or a pharmaceutically acceptable salt and/or solvate thereof) for use according to any one of embodiments 147, 150 or 152-174, or the use of the compound of Formula A (or a pharmaceutically acceptable salt and/or solvate thereof) according to any one of embodiments 148, 151 or 152-174,
   wherein about 50 μL to about 60 μL of the solution is administered per intravitreal administration.
176. The method according to any one of embodiments 146, 149 or 152-174, the compound of Formula A (or a pharmaceutically acceptable salt and/or solvate thereof) for use according to any one of embodiments 147, 150 or 152-174, or the use of the compound of Formula A (or a pharmaceutically acceptable salt and/or solvate thereof) according to any one of embodiments 148, 151 or 152-174,
   wherein about 60 μL to about 70 μL of the solution is administered per intravitreal administration.
177. The method according to any one of embodiments 146, 149 or 152-174, the compound of Formula A (or a pharmaceutically acceptable salt and/or solvate thereof) for use according to any one of embodiments 147, 150 or 152-174, or the use of the compound of Formula A (or a pharmaceutically acceptable salt and/or solvate thereof) according to any one of embodiments 148, 151 or 152-174,
   wherein about 70 μL to about 80 μL of the solution is administered per intravitreal administration.
178. The method according to any one of embodiments 146, 149 or 152-174, the compound of Formula A (or a pharmaceutically acceptable salt and/or solvate thereof) for use according to any one of embodiments 147, 150 or 152-174, or the use of the compound of Formula A (or a pharmaceutically acceptable salt and/or solvate thereof) according to any one of embodiments 148, 151 or 152-174,
   wherein about 80 μL to about 90 μL of the solution is administered per intravitreal administration.
179. The method according to any one of embodiments 146, 149 or 152-174, the compound of Formula A (or a pharmaceutically acceptable salt and/or solvate thereof) for use according to any one of embodiments 147, 150 or 152-174, or the use of the compound of Formula A (or a pharmaceutically acceptable salt and/or solvate thereof) according to any one of embodiments 148, 151 or 152-174,
   wherein about 90 μL to about 100 μL of the solution is administered per intravitreal administration.
180. The method according to any one of embodiments 146, 149 or 152-175, the compound of Formula A (or a pharmaceutically acceptable salt and/or solvate thereof) for use according to any one of embodiments 147, 150 or 152-175, or the use of the compound of Formula A (or a pharmaceutically acceptable salt and/or solvate thereof) according to any one of embodiments 148, 151 or 152-175,
   wherein about 50 μL of the solution is administered per intravitreal administration.
181. The method according to any one of embodiments 146, 149 or 152-176, the compound of Formula A (or a pharmaceutically acceptable salt and/or solvate thereof) for use according to any one of embodiments 147, 150 or 152-176, or the use of the compound of Formula A (or a pharmaceutically acceptable salt and/or solvate thereof) according to any one of embodiments 148, 151 or 152-176,
   wherein about 60 μL of the solution is administered per intravitreal administration.
182. The method according to any one of embodiments 146, 149 or 152-174 or 176-177, the compound of Formula A (or a pharmaceutically acceptable salt and/or solvate thereof) for use according to any one of embodiments 147, 150 or 152-174 or 176-177, or the use of the compound of Formula A (or a pharmaceutically acceptable salt and/or solvate thereof) according to any one of embodiments 148, 151 or 152-174 or 176-177,
   wherein about 70 μL of the solution is administered per intravitreal administration.
183. The method according to any one of embodiments 146, 149 or 152-174 or 177-178, the compound of Formula A (or a pharmaceutically acceptable salt and/or solvate thereof) for use according to any one of embodiments 147, 150 or 152-174 or 177-178, or the use of the compound of Formula A (or a pharmaceutically acceptable salt and/or solvate thereof) according to any one of embodiments 148, 151 or 152-174 or 177-178,
   wherein about 80 μL of the solution is administered per intravitreal administration.
184. The method according to any one of embodiments 146, 149 or 152-174 or 178-179, the compound of Formula A (or a pharmaceutically acceptable salt and/or solvate thereof) for use according to any one of embodiments 147, 150 or 152-174 or 178-179, or the use of the compound of Formula A (or a pharmaceutically acceptable salt and/or solvate thereof) according to any one of embodiments 148, 151 or 152-174 or 178-179,
   wherein about 90 μL of the solution is administered per intravitreal administration.
185. The method according to any one of embodiments 146, 149 or 152-174 or 179, the compound of Formula A (or a pharmaceutically acceptable salt and/or solvate thereof) for use according to any one of embodiments 147, 150 or 152-174 or 179, or the use of the compound of Formula A (or a pharmaceutically acceptable salt and/or solvate thereof) according to any one of embodiments 148,151 or 152-174 or 179,
   wherein about 100 μL of the solution is administered per intravitreal administration.
186. The method according to any one of embodiments 146, 149 or 152-185, the compound of Formula A (or a pharmaceutically acceptable salt and/or solvate thereof) for use according to any one of embodiments 147, 150 or 152-185, or the use of the compound of Formula A (or a pharmaceutically acceptable salt and/or solvate thereof) according to any one of embodiments 148,151 or 152-185,
   wherein prior to administration of the compound of Formula A, the patient has a baseline visual acuity score (BCVA) of between 19 and 73 letters in at least one eye, measured using the standard Early Treatment Diabetic Retinopathy Study (ETDRS) chart.
187. The method according to any one of embodiments 146, 149 or 152-186, the compound of Formula A (or a pharmaceutically acceptable salt and/or solvate thereof) for use according to any one of embodiments 147, 150 or 152-186, or the use of the compound of Formula A (or a pharmaceutically acceptable salt and/or solvate thereof) according to any one of embodiments 148,151 or 152-186,
   wherein the patient is in the early stages of DME or impaired visual acuity.
188. The method according to embodiment 187, the compound of Formula A (or a pharmaceutically acceptable salt and/or solvate thereof) for use according to embodiment 187, or the use of the compound of Formula A (or a pharmaceutically acceptable salt and/or solvate thereof) according to embodiment 187,
   wherein a patient in the early stages of DME or impaired visual acuity is defined by having a baseline visual acuity score (BCVA), prior to administration of the compound of Formula A, of between 56 and 73 letters in at least one eye, measured using the standard Early Treatment Diabetic Retinopathy Study (ETDRS) chart.
189. The method according to any one of embodiments 146, 149 or 152-188, the compound of Formula A (or a pharmaceutically acceptable salt and/or solvate thereof) for use according to any one of embodiments 147, 150 or 152-188, or the use of the compound of Formula A (or a pharmaceutically acceptable salt and/or solvate thereof) according to any one of embodiments 148,151 or 152-188,
   wherein the first time period is greater than about 8 weeks.
190. The method according to any one of embodiments 146, 149 or 152-189, the compound of Formula A (or a pharmaceutically acceptable salt and/or solvate thereof) for use according to any one of embodiments 147, 150 or 152-189, or the use of the compound of Formula A (or a pharmaceutically acceptable salt and/or solvate thereof) according to any one of embodiments 148,151 or 152-189,
   wherein the first time period is greater than about 12 weeks.
191. The method according to any one of embodiments 146, 149 or 152-189, the compound of Formula A (or a pharmaceutically acceptable salt and/or solvate thereof) for use according to any one of embodiments 147, 150 or 152-189, or the use of the compound of Formula A (or a pharmaceutically acceptable salt and/or solvate thereof) according to any one of embodiments 148,151 or 152-189,
   wherein the first time period between about 10 weeks and about 12 weeks.
192. The method according to any one of embodiments 146, 149 or 152-189, the compound of Formula A (or a pharmaceutically acceptable salt and/or solvate thereof) for use according to any one of embodiments 147, 150 or 152-189, or the use of the compound of Formula A (or a pharmaceutically acceptable salt and/or solvate thereof) according to any one of embodiments 148,151 or 152-189,
   wherein the first time period is about 12 weeks.
193. The method according to any one of embodiments 146, 149 or 152-192, the compound of Formula A (or a pharmaceutically acceptable salt and/or solvate thereof) for use according to any one of embodiments 147, 150 or 152-192, or the use of the compound of Formula A (or a pharmaceutically acceptable salt and/or solvate thereof) according to any one of embodiments 148,151 or 152-192,
   wherein the first dosing frequency is between about once every three weeks and about once every five weeks.
194. The method according to any one of embodiments 146, 149 or 152-193, the compound of Formula A (or a pharmaceutically acceptable salt and/or solvate thereof) for use according to any one of embodiments 147, 150 or 152-193, or the use of the compound of Formula A (or a pharmaceutically acceptable salt and/or solvate thereof) according to any one of embodiments 148,151 or 152-193,
   wherein the second time period is greater than about 8 weeks.
195. The method according to any one of embodiments 146, 149 or 152-194, the compound of Formula A (or a pharmaceutically acceptable salt and/or solvate thereof) for use according to any one of embodiments 147, 150 or 152-194, or the use of the compound of Formula A (or a pharmaceutically acceptable salt and/or solvate thereof) according to any one of embodiments 148,151 or 152-194,
   wherein the second time period is greater than about 12 weeks.
196. The method according to any one of embodiments 146, 149 or 152-195, the compound of Formula A (or a pharmaceutically acceptable salt and/or solvate thereof) for use according to any one of embodiments 147, 150 or 152-195, or the use of the compound of Formula A (or a pharmaceutically acceptable salt and/or solvate thereof) according to any one of embodiments 148,151 or 152-195,
   wherein the second time period is greater than about 16 weeks
197. The method according to any one of embodiments 146, 149 or 152-194, the compound of Formula A (or a pharmaceutically acceptable salt and/or solvate thereof) for use according to any one of embodiments 147, 150 or 152-194, or the use of the compound of Formula A (or a pharmaceutically acceptable salt and/or solvate thereof) according to any one of embodiments 148,151 or 152-194,
   wherein the second time period is between about 8 weeks and about 12 weeks.
198. The method according to any one of embodiments 146, 149 or 152-194, the compound of Formula A (or a pharmaceutically acceptable salt and/or solvate thereof) for use according to any one of embodiments 147, 150 or 152-194, or the use of the compound of Formula A (or a pharmaceutically acceptable salt and/or solvate thereof) according to any one of embodiments 148,151 or 152-194,
   wherein the second time period is about 12 weeks.
199. The method according to any one of embodiments 146, 149 or 152-198, the compound of Formula A (or a pharmaceutically acceptable salt and/or solvate thereof) for use according to any one of embodiments 147, 150 or 152-198, or the use of the compound of Formula A (or a pharmaceutically acceptable salt and/or solvate thereof) according to any one of embodiments 148,151 or 152-198,
   wherein the second dosing frequency is lower than about once every six weeks.
200. The method according to any one of embodiments 146, 149 or 152-199, the compound of Formula A (or a pharmaceutically acceptable salt and/or solvate thereof) for use according to any one of embodiments 147, 150 or 152-199, or the use of the compound of Formula A (or a pharmaceutically acceptable salt and/or solvate thereof) according to any one of embodiments 148,151 or 152-199,
   wherein the second dosing frequency is lower than about once every eight weeks.
201. The method according to any one of embodiments 146, 149 or 152-200, the compound of Formula A (or a pharmaceutically acceptable salt and/or solvate thereof) for use according to any one of embodiments 147, 150 or 152-200, or the use of the compound of Formula A (or a pharmaceutically acceptable salt and/or solvate thereof) according to any one of embodiments 148,151 or 152-200,
   wherein the second dosing frequency is lower than about once every twelve weeks.
202. The method according to any one of embodiments 146, 149 or 152-201, the compound of Formula A (or a pharmaceutically acceptable salt and/or solvate thereof) for use according to any one of embodiments 147, 150 or 152-201, or the use of the compound of Formula A (or a pharmaceutically acceptable salt and/or solvate thereof) according to any one of embodiments 148,151 or 152-201,
   wherein the solution further comprises at least one non-ionic tonicity agent.
203. The method according to embodiment 202, the compound of Formula A (or a pharmaceutically acceptable salt and/or solvate thereof) for use according to embodiment 202, or the use of the compound of Formula A (or a pharmaceutically acceptable salt and/or solvate thereof) according to embodiment 202,
   wherein the at least one non-ionic tonicity agent is trehalose.
204. The method according to embodiment 203, the compound of Formula A (or a pharmaceutically acceptable salt and/or solvate thereof) for use according to embodiment 203, or the use of the compound of Formula A (or a pharmaceutically acceptable salt and/or solvate thereof) according to embodiment 203,
   wherein the trehalose is provided as trehalose dihydrate.
205. The method according to any one of embodiments 146, 149 or 152-204, the compound of Formula A (or a pharmaceutically acceptable salt and/or solvate thereof) for use according to any one of embodiments 147, 150 or 152-204, or the use of the compound of Formula A (or a pharmaceutically acceptable salt and/or solvate thereof) according to any one of embodiments 148,151 or 152-204,
   wherein the solution further comprises histidine.
206. The method according to any one of embodiments 146, 149 or 152-205, the compound of Formula A (or a pharmaceutically acceptable salt and/or solvate thereof) for use according to any one of embodiments 147, 150 or 152-205, or the use of the compound of Formula A (or a pharmaceutically acceptable salt and/or solvate thereof) according to any one of embodiments 148,151 or 152-205,
   wherein the pharmaceutical composition comprises an aqueous solution of the compound of formula A (or a pharmaceutically acceptable salt and/or solvate thereof), histidine and trehalose dihydrate.
207. The method according to any one of embodiments 146, 149 or 152-206, the compound of Formula A (or a pharmaceutically acceptable salt and/or solvate thereof) for use according to any one of embodiments 147, 150 or 152-206, or the use of the compound of Formula A (or a pharmaceutically acceptable salt and/or solvate thereof) according to any one of embodiments 148,151 or 152-206,
   wherein the pharmaceutical composition has a pH from about 2 to about 10, preferably from about 5 to about 7.5, preferably from about 5.3 to about 6, and preferably from about 5.4 to about 5.8.
208. The method according to any one of embodiments 146, 149 or 152-207, the compound of Formula A (or a pharmaceutically acceptable salt and/or solvate thereof) for use according to any one of embodiments 147, 150 or 152-207, or the use of the compound of Formula A (or a pharmaceutically acceptable salt and/or solvate thereof) according to any one of embodiments 148,151 or 152-207,
   wherein the pharmaceutical composition has a pH of about 5.5.
209. The method according to any one of embodiments 146, 149 or 152-208, the compound of Formula A (or a pharmaceutically acceptable salt and/or solvate thereof) for use according to any one of embodiments 147, 150 or 152-208, or the use of the compound of Formula A (or a pharmaceutically acceptable salt and/or solvate thereof) according to any one of embodiments 148,151 or 152-208,
   wherein the treatment is a monotherapy for DME or impaired visual acuity.
210. The method according to any one of embodiments 146, 149 or 152-209, the compound of Formula A (or a pharmaceutically acceptable salt and/or solvate thereof) for use according to any one of embodiments 147, 150 or 152-209, or the use of the compound of Formula A (or a pharmaceutically acceptable salt and/or solvate thereof) according to any one of embodiments 148,151 or 152-209, wherein the patient has previously had anti-VEGF (vascular endothelial growth factor) treatment.
211. The method according to embodiment 210, the compound of Formula A (or a pharmaceutically acceptable salt and/or solvate thereof) for use according to embodiment 210, or the use of the compound of Formula A (or a pharmaceutically acceptable salt and/or solvate thereof) according to embodiment 210,
   wherein the anti-VEGF treatment is selected from aflibercept (Eylea®), bevacizumab, ranibizumab, and pegaptanib.
212. The method according to any one of embodiments 210-211, the compound of Formula A (or a pharmaceutically acceptable salt and/or solvate thereof) for use according to any one of embodiments 210-211, or the use of the compound of Formula A (or a pharmaceutically acceptable salt and/or solvate thereof) according to any one of embodiments 210-211,
   wherein the anti-VEGF is aflibercept (Eylea®).
213. The method according to any one of embodiments 210-212, the compound of Formula A (or a pharmaceutically acceptable salt and/or solvate thereof) for use according to any one of embodiments 210-212, or the use of the compound of Formula A (or a pharmaceutically acceptable salt and/or solvate thereof) according to any one of embodiments 210-212,
   wherein the patient received anti-VEGF treatment for no more than 36 months before commencing treatment with the compound of Formula A (or a pharmaceutically acceptable salt and/or solvate thereof).
214. The method according to any one of embodiments 210-213, the compound of Formula A (or a pharmaceutically acceptable salt and/or solvate thereof) for use according to any one of embodiments 210-213, or the use of the compound of Formula A (or a pharmaceutically acceptable salt and/or solvate thereof) according to any one of embodiments 210-213,
   wherein the patient received anti-VEGF treatment no less than 8 weeks before commencing treatment with the compound of formula A (or a pharmaceutically acceptable salt and/or solvate thereof).
215. The method according to any one of embodiments 146, 149 or 152-215, the compound of Formula A (or a pharmaceutically acceptable salt and/or solvate thereof) for use according to any one of embodiments 147, 150 or 152-215, or the use of the compound of Formula A (or a pharmaceutically acceptable salt and/or solvate thereof) according to any one of embodiments 148,151 or 152-215,
   wherein the patient does not receive anti-VEGF treatment concurrent to administration of the compound of Formula A.
216. The method according to any one of embodiments 1, 4, 7-75, 119-123, or 210-214; or any one of embodiments 124-145 when dependent on any one of embodiments 119-123; or embodiment 215 when dependent on any one of embodiments 119-123;
   the compound of Formula A (or a pharmaceutically acceptable salt and/or solvate thereof) for use according to any one of embodiments 2, 5, 7-75, 119-123, or 210-214; or any one of embodiments 124-145 when dependent on any one of embodiments 119-123; or embodiment 215 when dependent on any one of embodiments 119-123; or the use of the compound of Formula A (or a pharmaceutically acceptable salt and/or solvate thereof) according to any one of embodiments 3, 6, 7-75, 119-123, or 210-214; or any one of embodiments 124-145 when dependent on any one of embodiments 119-123; or embodiment 215 when dependent on any one of embodiments 119-123;
   wherein the previous anti-VEGF treatment was for treating impaired visual acuity or DME.
217. The method according to any one of embodiments 1, 4, 7-75, 76, 79, 82-145, 146, 149 or 152-216, the compound of Formula A (or a pharmaceutically acceptable salt and/or solvate thereof) for use according to any one of embodiments 2, 5, 7-75, 77, 80, 82-145, 147, 150 or 152-216, or the use of the compound of Formula A (or a pharmaceutically acceptable salt and/or solvate thereof) according to any one of embodiments 3, 6, 7-75, 78, 81, 82-145, 148, 151 or 152-216,
   wherein the treatment with the solution comprising the compound of Formula A (or a pharmaceutically acceptable salt and/or solvate thereof) slows the progression of impaired visual acuity or DME.
218. The method according to any one of embodiments 1, 4 or 7-53, any one of embodiments 55-75 when not dependent on embodiment 54, any one of embodiments 76, 79 or 82-123, any one of embodiments 125-145 when not dependent on embodiment 124, any one of embodiments 146, 149 or 152-214, or any one of embodiments 216 or 217 when not dependent on any of embodiments 54, 124 or 215, or
   the compound of Formula A (or a pharmaceutically acceptable salt and/or solvate thereof) for use according to any one of embodiments 2, 5 or 7-53, any one of embodiments 55-75 when not dependent on embodiment 54, any one of embodiments 77, 80 or 82-123, any one of embodiments 125-145 when not dependent on embodiment 124, any one of embodiments 147, 150 or 152-214, or any one of embodiments 216 or 217 when not dependent on any of embodiments 54, 124 or 215, or
   the use of the compound of Formula A (or a pharmaceutically acceptable salt and/or solvate thereof) according to any one of embodiments 3, 6 or 7-53, any one of embodiments 55-75 when not dependent on embodiment 54, any one of embodiments 78, 81 or 82-123, any one of embodiments 125-145 when not dependent on embodiment 124, any one of embodiments 148, 151 or 152-214, or any one of embodiments 216 or 217 when not dependent on any of embodiments 54, 124 or 215,
   wherein the patient receives anti-VEGF treatment in combination with the administration of the compound of Formula A (or a pharmaceutically acceptable salt and/or solvate thereof).
219. The method according to embodiment 218, the compound of Formula A (or a pharmaceutically acceptable salt and/or solvate thereof) for use according to embodiment 218, or the use of the compound of Formula A (or a pharmaceutically acceptable salt and/or solvate thereof) according to embodiment 218,
   wherein the anti-VEGF treatment received in combination is administered in the same pharmaceutical composition as the compound of Formula A (or a pharmaceutically acceptable salt and/or solvate thereof).
220. The method according to embodiment 218, the compound of Formula A (or a pharmaceutically acceptable salt and/or solvate thereof) for use according to embodiment 218, or the use of the compound of Formula A (or a pharmaceutically acceptable salt and/or solvate thereof) according to embodiment 218,
   wherein the anti-VEGF treatment received in combination is administered in a different pharmaceutical composition to the compound of Formula A (or a pharmaceutically acceptable salt and/or solvate thereof).
221. The method according to embodiment 220, the compound of Formula A (or a pharmaceutically acceptable salt and/or solvate thereof) for use according to embodiment 220, or the use of the compound of Formula A (or a pharmaceutically acceptable salt and/or solvate thereof) according to embodiment 220,
   wherein the different pharmaceutical compositions is administered separately, sequentially or simultaneously.
222. The method according to any one of embodiments 218-221, the compound of Formula A (or a pharmaceutically acceptable salt and/or solvate thereof) for use according to any one of embodiments 218-221, or the use of the compound of Formula A (or a pharmaceutically acceptable salt and/or solvate thereof) according to any one of embodiments 218-221,
   wherein the anti-VEGF treatment received in combination is selected from aflibercept (Eylea®), bevacizumab, ranibizumab, and pegaptanib.

### BRIEF DESCRIPTION OF DRAWINGS

FIG. 1 is a graphical representation of the change in BCVA letters versus sham over time for 3 μg of compound of Formula A and 6 μg of compound of Formula A; and

FIG. 2 is a graphical representation of the change in BCVA letters versus sham over time for early stage compared with all subjects on the dose of 6 μg of compound of Formula A.

### MODES FOR CARRYING OUT THE INVENTION

The invention is further illustrated by the following examples. It will be appreciated that the examples are for illustrative purposes only and are not intended to limit the invention as described above. Modification of detail may be made without departing from the scope of the invention. In the following examples, the following abbreviations and definitions are used:

| | |
|---|---|
| AE | Adverse event |
| ANCOVA | Analysis of covariance |
| Aq | Aqueous solution |
| ASNV | Anterior segment neovascularization |
| BCVA | Best corrected visual acuity |
| BMI | Body mass index |
| BP | Blood pressure |
| C1-INH | C1-esterase Inhibitor |
| ciDME | Center-involving diabetic macular edema |
| CIRC | Central Image Reading Center |
| CST | Central subfield thickness |
| DBP | Diastolic blood pressure |
| DRSS | Diabetic Retinopathy Severity Scale |
| DM | Diabetes Mellitus |
| DME | Diabetic macular edema |
| DRL | Drug Reference List |
| ED | Early discontinuation |
| ETDRS | Early Treatment Diabetic Retinopathy Study |
| FAS | Full analysis set |
| FE | Fellow eye |
| GCP | Good Clinical Practice |
| HgA1c | Glycosylated haemoglobin |
| HMWK | High-molecular-weight kininogen |
| Hrs | Hours |
| IB | Investigator's Brochure |
| ICF | Informed consent form |
| IEC | Independent Ethics Committee |
| IOP | Intraocular pressure |
| IPA | iso-propanol |
| IRB | Institutional review board |
| IRT | Interactive response technology |
| ITT | Intention-to-treat |
| IVT | Intravitreal |
| LOCF | Last observation carried forward |
| Me | Methyl |
| MeCN | Acetonitrile |
| MedDRA | Medical Dictionary for Regulatory Activities |
| MeOH | Methanol |
| Min | Minutes |
| NOAEL | No-observed adverse effect level |
| NSAID | Nonsteroidal anti-inflammatory drug |
| OCT | Optical coherence tomography |
| PDR | Proliferative diabetic retinopathy |
| Ph | Phenyl |
| PKal | Plasma kallikrein |
| PPS | Per protocol set |
| PR | Pulse rate |
| QS | Quantum satis (sufficient quantity) |
| RRT | Relative retention time |
| rt | room temperature |
| RVP | Retinal vascular permeability |
| SAE | Serious adverse event |
| SAF | Safety set |
| SAP | Statistical analysis plan |
| SBP | Systolic blood pressure |
| SD-OCT | Spectral-domain optical coherence tomography |
| SD | Standard deviation |
| SE | Study eye |
| SUSAR | Serious unexpected suspected adverse reaction |
| SWFI | Sterile water for injection |
| TEAE | Treatment-emergent adverse event |
| US | United States |
| VA | Visual acuity |
| VEGF | Vascular endothelial growth factor |
| WHO | World Health Organisation |

Osmolality was determined using a calibrated osmometer in compliance with USP<785> (freezing point depression). (See United States Pharmacopeia (USP) 37, NF 32).

Particulate matter in the pharmaceutical compositions was measured using the microscopic particle count test described in USP <789> (Particulate matter in ophthalmic solutions) (See United States Pharmacopeia (USP) 37, NF 32).

### Synthetic Examples

The compound of Formula A may be prepared according to the method described in Evans et al. (“Benzylamine derivatives as inhibitors of plasma kallikrein” WO2013/005045). N—[(R)-1-[(S)-1-(4-Aminomethyl-benzylcarbamoyl)-2-phenyl-ethylcarbamoyl]-2-(4-ethoxy-phenyl)-ethyl]-benzamide hydrochloride, the hydrochloride salt of the compound of Formula A, can be manufactured using methods disclosed in WO2014/006414. The structure of the compound of Formula A is shown below:

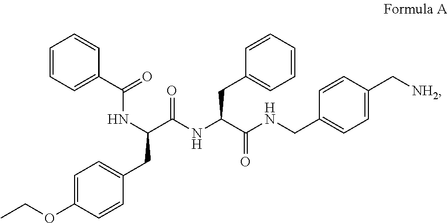 [SMILES: CCOc1ccc(C[C@@H](NC(=O)c2ccccc2)C(=O)N[C@@H](Cc2ccccc2)C(=O)NCc2ccc(CN)cc2)cc1]

### Solid Forms and Concentrations

Concentrations and dose levels defined in the examples below are based on the amount of free base of the compound of Formula A.

As outlined below, the compound of Formula A is prepared as a solution formulation. Therefore, any solid form of the compound of Formula A may be used in preparing the solution formulation.

It would readily understood that the invention is not limited to the use of specific solid forms and any other solid form could also be used to prepare solution formulations of the compound of Formula A.

Preparative Compositions of 10, 30, 100 and 300 μg/mL Solution Formulations of the Compound of Formula a

A 9.8% w/w trehalose and 2 mM histidine buffer solution is prepared by dissolving L-histidine (1.09 g) and trehalose dihydrate (356.7 g) in SWFI (3270 g) with agitation. The buffer pH is adjusted using 1.0N HCl solution as needed and diluted to 3640 g with SWFI to yield the buffer solution. Compound of Formula A (0.340 g) is dissolved in the trehalose-histidine buffer (2800 g) solution with high energy rotor stator mixing at 40° C. for sufficient time to provide a visibly clear, colorless solution, approximately 15-30 min. The pH of the solution is adjusted as needed with 1.0N HCl solution. HPLC is used to determine concentration of the compound of Formula A in the solution and the solution is diluted as needed with the trehalose-histidine buffer solution. The resulting 100 μg/mL solution formulation of the compound of Formula A is sterile filtered through two PVDF sterile filtration modules in series into a sterile, depyrogenated pyrex glass container.

10, 30 and 300 μg/mL solution formulations of the compound of Formula A were prepared analogously with a common buffer and with the amount of the compound of Formula A being varied. For example, 0.104 g of the compound of Formula A was used to prepare the 30 μg/mL solution and 0.0363 g of the compound of Formula A was used to prepare the 10 μg/mL solution formulations.

Table 1 below provides analytical and characterization data for the 10, 30 and 100 μg/mL solution formulations of the compound of Formula A.

TABLE 1
Analytical and characterization data for the 10, 30, 100 and
300 μg/mL solution formulations of the compound of Formula A

| | | 10 μg/mL | 30 μg/mL | 100 μg/mL | 300 μg/mL |
|---|---|---|---|---|---|
| Appearance* | | C, C, L, | C, C, L, | C, C, L, | C, C, L, |
| | | FVP | FVP | FVP | FVP |
| Assay | | 108 | 106 | 103 | 107 |
| (% LC)** | | | | | |
| Purity | | 99.9 | 99.9 | 100 | 100 |
| (area %) | | | | | |
| Impurities*** | | RRT 0.64- | RRT 0.57- | ND | ND |
| | | 0.17% | 0.11% | | |
| PH | | 5.8 | 5.5 | 5.5 | 5.6 |
| Osmolality | | 304 | 302 | 303 | 307 |
| (mOsmol/Kg) | | | | | |
| | ≥10 μm | 0.1 | 0.1 | 0.1 | 0.4 |
| | ≥25 μm | 0.0 | 0.1 | 0.0 | 0.15 |
| | ≥50 μm | 0.0 | 0.0 | 0.0 | 0.0 |

| | | | | |
|---|---|---|---|---|
| Bacterial | <0.0500 | <0.0500 | <0.0500 | <0.0500 |
| endotoxin | | | | |
| (EU/mL) | | | | |
| Sterility | Sterile | Sterile | Sterile | Sterile |

*C, C, L, FVP = Clear, Colorless, Liquid, Free from Visible Particles
**% LC = % Label Claim
***ND = not detected

The 10, 30, 100 and 300 μg/mL solution formulations of the compound of Formula A are stable when filled into 2 mL clear type 1 glass vials sealed with chlorobutyl rubber stoppers, as shown by the data in Table 2

TABLE 2
Stability data for the 10, 30, 100 and 300 μg/mL solution formulations of the compound of Formula A

| | | 10 μg/mL | | 30 μg/mL | | 100 μg/mL | | 300 μg/mL | |
|---|---|---|---|---|---|---|---|---|---|
| | | | 36 | | 36 | | 36 | | 36 |
| | | | months | | months | | months | | months |
| | | | at 25° C. | | at 25° C. | | at 25° C. | | at 25° C. |
| | | | and 60% | | and 60% | | and 60% | | and 60% |
| | | Initial | RH⁺ | Initial | RH⁺ | Initial | RH⁺ | Initial | RH⁺ |
| Appearance* | | C, C, | C, C, | C, C, | C, C, | C, C, | C, C, | C, C, | C, C, |
| | | L, FVP | L, FVP | L, FVP | L, FVP | L, FVP | L, FVP | L, FVP | L, FVP |
| Assay (% LC)** | | 108 | 109 | 106 | 103 | 103 | 101 | 107 | 106 |
| Purity (area %) | | 99.9 | 99.7 | 99.9 | 99.9 | 100 | 99.8 | 100 | 99.9 |
| Impurities | | | | | | | | | |
| RRT 0.32 | | — | — | — | 0.03% | — | — | — | — |
| RRT 0.56-0.58 | | — | 0.10% | 0.11% | 0.04% | — | 0.04% | — | — |
| RRT 0.59-0.60 | | — | 0.06% | — | — | — | — | — | — |
| RRT 0.63-0.65 | | 0.17% | 0.05% | — | — | — | — | — | — |
| RRT 0.69 | | — | 0.05% | — | 0.07% | — | 0.06% | — | 0.06% |
| RRT 0.83 | | — | — | — | 0.03% | — | — | — | — |
| RRT 1.34 | | — | 0.04% | — | — | — | 0.06% | — | 0.04% |
| RRT 1.39 | | — | — | — | — | — | 0.04% | — | 0.03% |
| pH | | 5.8 | 5.9 | 5.5 | 5.6 | 5.5 | 5.6 | 5.6 | 5.7 |
| Osmolality | | 304 | 307 | 302 | 307 | 303 | 305 | 307 | 308 |
| (mOsmol/Kg) | | | | | | | | | |
| Particulate | ≥10 μm | 0.1 | 0.25 | 0.1 | 0.25 | 0.1 | 0.2 | 0.4 | 0.6 |
| matter/mL | ≥25 μm | 0.0 | 0.2 | 0.1 | 0.2 | 0.0 | 0.2 | 0.15 | 0.55 |
| | ≥50 μm | 0.0 | 0.1 | 0.0 | 0.15 | 0.0 | 0.15 | 0.0 | 0.35 |

| | | | | | | | | |
|---|---|---|---|---|---|---|---|---|
| Bacterial | <0.0500 | <0.0500 | <0.0500 | <0.0500 | <0.0500 | <0.0500 | <0.0500 | <0.0500 |
| endotoxin | | | | | | | | |
| (EU/mL) | | | | | | | | |
| Sterility | Sterile | Sterile | Sterile | Sterile | Sterile | Sterile | Sterile | Sterile |

*C, C, L, FVP = Clear, Colorless, Liquid, Free from Visible Particles
**% LC = % Label Claim
⁺RH = relative humidity

### Background Example 2

Preparative Compositions of 30, 60 and 200 μg/mL Solution Formulations of the Compound of Formula A

A 9.8% w/w trehalose and 2 mM histidine buffer solution is prepared by dissolving L-histidine monohydrochloride monohydrate (1.33 g), trehalose dihydrate (407.7 g) and L-histidine (0.26 g) in SWFI (3536 g) with agitation. Additional SWFI is added to bring the weight to 4160 g and the mixture agitated. N—[(R)-1-[(S)-1-(4-aminomethyl-benzylcarbamoyl)-2-phenyl-ethylcarbamoyl]-2-(4-ethoxy-phenyl)-ethyl]-benzamide hydrochloride (the hydrochloride salt of the compound of Formula A) (0.066 g) is dissolved in the trehalose-histidine buffer (2080 g) solution with high energy rotor stator mixing at 50° C. for sufficient time to provide a visibly clear, colorless solution, approximately 15-30 min. The pH of the solution is adjusted as needed with 1.0N HCl solution. HPLC is used to determine concentration of the compound of Formula A in the solution and the solution is diluted as needed with the trehalose-histidine buffer solution. The resulting 30 μg/mL solution formulation of the compound of Formula A is sterile filtered through two PVDF sterile filtration modules in series into a sterile, depyrogenated pyrex glass container.

60 μg/mL solution formulation of the compound of Formula A was prepared analogously with a common buffer and with 0.131 g of the compound of Formula A being used instead. 200 μg/mL solution formulation of the compound of Formula A was prepared analogously with a common buffer and with 0.436 g of the compound of Formula A being used instead.

Table 3 below provides analytical and characterization data for the 30, 60 and 200 μg/mL μg/mL solution formulations of the compound of Formula A.

TABLE 3
Analytical and characterization data for the 30, 60 and 200
μg/mL solution formulations of the compound of Formula A

| | | 30 μg/mL | 60 μg/mL | 200 μg/mL |
|---|---|---|---|---|
| Appearance * | | C, C, L, FVP | C, C, L, FVP | C, C, L, FVP |
| Assay (% LC) ** | | 95 | 95 | 195 |
| Purity (area %) | | 99.9 | 99.8 | 100.0 |
| Impurities *** | | RRT 0.57: 0.13% | RRT 0.57: 0.22% | ND |
| pH | | 5.4 | 5.5 | 5.7 |
| Osmolality (mOsmol/kg) | | 307 | 304 | Not tested |
| Particulate | ≥10 μm | 0.1 | 0.15 | Not tested |
| matter/mL | ≥25 μm | 0.1 | 0.3 | Not tested |
| | ≥50 μm | 0.05 | 0.15 | Not tested |

| | | | |
|---|---|---|---|
| Bacterial endotoxin (EU/mL) | <0.0500 | <0.0500 | Not tested |
| Sterility | Sterile | Sterile | Not tested |

* C, C, L, FVP = Clear, Colorless, Liquid, Free from Visible Particles
** % LC = % Label Claim
*** ND = not detected

TABLE 4
Stability data for the 30, 60 and 200 μg/mL
solution formulations of the compound of Formula A

| | | 30 μg/mL | | 60 μg/mL | | 200 μg/mL | |
|---|---|---|---|---|---|---|---|
| | | | 24 months | | 24 months | | 24 months |
| | | | at 25° | | at 25° | | at 25° |
| | | | C. and | | C. and | | C. and |
| | | Initial | 60% RH⁺ | Initial | 60% RH⁺ | Initial | 60% RH⁺ |
| Appearance* | | C, C, | C, C, | C, C, | C, C, | C, C, | C, C, |
| | | L, FVP | L, FVP | L, FVP | L, FVP | L, FVP | L, FVP |
| Assay (% LC)** | | 96 | 94 | 99 | 96 | 98 | 96 |
| Purity (area %) | | 100.0 | 99.9 | 100.0 | 99.9 | 100.0 | 99.8 |
| Impurities | | | | | | | |
| RRT 0.56-0.58 | | — | — | 0.06 | — | — | 0.02 |
| RRT 0.69-0.70 | | 0.04 | 0.07 | — | 0.08 | — | 0.05 |
| RRT 0.94 | | — | — | — | — | — | 0.02 |
| RRT 1.12 | | — | — | — | — | — | 0.03 |
| RRT 1.14 | | — | — | — | — | — | 0.03 |
| RRT 1.33 | | — | — | — | — | — | 0.11 |
| pH | | 5.5 | 5.5 | 5.4 | 5.5 | 5.7 | 5.7 |
| Osmolality | | 308 | 310 | 306 | 311 | Not tested | Not tested |
| (mOsmol/Kg) | | | | | | | |
| Particulate | ≥10 μm | 0.25 | 0.25 | 0.1 | 0.3 | Not tested | Not tested |
| matter/mL | ≥25 μm | 0.15 | 0.15 | 0.1 | 0.25 | Not tested | Not tested |
| | ≥50 μm | 0.1 | 0.1 | 0.05 | 0.15 | Not tested | Not tested |

| | | | | | | |
|---|---|---|---|---|---|---|
| Bacterial | <0.0500 | <0.0500 | <0.0500 | <0.0500 | Not tested | Not tested |
| endotoxin | | | | | | |
| (EU/mL) | | | | | | |
| Sterility | Sterile | Sterile | Sterile | Sterile | Not tested | Not tested |

*C, C, L, FVP = Clear, Colorless, Liquid, Free from Visible Particles
**% LC = % Label Claim
⁺RH = relative humidity

### Study 1:

Study—an Open Label, Single Ascending Dose Study to Investigate the Safety and Tolerability of the Compound of Formula A Administered by Intravitreal Injection in Subjects with Center Involved Diabetic Macular Edema and Reduced Vision

The primary objective of the trial was to evaluate the local and systemic safety and tolerability of single ascending doses administered via intravitreal injection of the compound of Formula A in adult male and female subjects with central involved diabetic macular edema.

The secondary objectives were:
To evaluate the plasma profile of the compound of Formula A following intravitreal injection in adult male and female subjects with central involved diabetic macular edema.
To evaluate the pharmacodynamic effect of intravitreal injection of the compound of Formula A in adult male and female subjects with central involved diabetic macular edema.

### Methodology:

Part 1 of the study had a single ascending dose design with up to 4 groups, with 3 subjects per group.

Following the signing of informed consent, subjects attended the clinic where screening assessments were recorded (Clinic Visit 1). When deemed eligible, subjects attended the clinic where baseline measurements were recorded (recorded as Day 0, Clinic Visit 2). Following confirmation of eligibility and the taking of baseline ophthalmic measurements, a single intravitreal injection of the compound of Formula A, was administered to the study eye as per Diabetic Retinopathy Clinical Research Network (DRCR.net) protocol.

Each subject returned on Day 1, 7, 14, 28 and 56 for safety and ophthalmic assessment. Pharmacokinetic assessment was also taken. In addition, the subject was contacted by the Study Site on Day 3 (+1-1 day) by telephone to inquire about the subject's visual wellbeing and to ask about any adverse events.

An independent Data and Safety Monitoring Committee (DSMC) oversaw the conduct of the study.

Once the highest safe and tolerated (or practical) dose had been established, the study proceeded to Part 2 in which 5 additional subjects were treated with the established highest safe and tolerated (or practical) dose according to the same protocol with the same procedures performed as described for subjects enrolled into Part 1.

### Main Criteria for Eligibility:

### Inclusion Criteria

1. Male or female adult subjects 18 years of age and older
2. Confirmed diagnosis of Type I or Type II diabetes mellitus
3. Best corrected visual acuity, using Early Treatment Diabetic Retinopathy Study (ETDRS) electronic visual acuity (EVA) testing, of between 20/40 and 20/400 (Snellen equivalent) in the study eye
4. Fellow eye acuity 20/80 or better measured as above with no expectation of requirement for anti-vascular endothelial growth factor (anti-VEGF) treatment in fellow eye within 2 months of study drug administration
5. Presence of central involved DME in the study eye defined as Heidelberg Spectralis Optical Coherence Tomography (OCT) Central Subfield Thickness (CST) 305 μm in women and 320 μm in men in the study eye
6. Subjects who fulfil one of the following criteria:
   a. Subjects who have not previously received an anti-VEGF treatment and who, in the view of the Investigator, can have initiation of anti-VEGF treatment in the study eye deferred for at least 2 months following the date of anticipated study drug administration
   b. Subjects who are receiving regular anti-VEGF intravitreal injections who:
      i. Have received at least 3 intravitreal injections of an anti-VEGF treatment within the last 5 months (study drug administration will be at least 6 weeks after the most recent intravitreal administration of anti-VEGF) and
      ii. In the view of the Investigator, can have continuation of anti-VEGF treatment in the study eye deferred for at least 2 months following the date of anticipated study drug administration
   c. Subjects who have received anti-VEGF in the past (>3 months prior to study inclusion) but are not actively receiving treatment and who in the view of the Investigator, can have resumption of anti-VEGF or alternative treatment in the study eye deferred for at least 2 months following anticipated study drug administration
7. Subjects, who in the view of the Investigator, are not expected to require panretinal laser photocoagulation or intravitreal steroids or intraocular surgery in the study eye for at least 2 months following anticipated study drug administration
8. No prior treatment with panretinal photocoagulation in the study eye within the previous 3 months (prior focal/grid macular photocoagulation is allowed)
9. No prior treatment with intravitreal steroid in the study eye within the previous 3 months
10. No prior treatment with systemic corticosteroids or systemic anti-VEGF therapy within the previous 3 months
11. No prior vitrectomy in the study eye
12. No prior intraocular surgery in the study eye within the previous 3 months
13. For women, post-menopausal, surgically sterile, or agreeable to using highly effective contraception (highly effective means of contraception include use of 2 of the following: hormonal contraceptives (oral, implant, transdermal patch, or injection) at a stable dose for at least 3 months prior to Screening, barrier (condom with spermicide, diaphragm with spermicide, IUD) until 2 months after last dose of study medication)
14. No ocular disease in the study eye that in the opinion of the Investigator would impact the progression of DME or response to treatments for DME, e.g., extensive macular scarring, active inflammation, ocular or periocular infection, retinal detachment, aphakia, vitreomacular traction or substantial center-involving epiretinal membrane etc.
15. Ability and willingness, in the opinion of the Investigator, to comply with study procedures, follow up visits and obtain usable OCT scans including not expecting to relocate outside the study covered area during the course of the study
16. Electrocardiogram (ECG) recording without signs of clinically relevant pathology as determined by an appropriately qualified physician, in particular QTcF (Fridericia's correction) less than 450 ms for men and 470 ms for women (there will be central ECG reading)
17. Values for hematology and biochemistry tests of blood and urine showing no clinically relevant deviation as judged by an appropriately qualified physician.
18. Study participant voluntarily agrees to participate in this study and signs the Institutional Review Board (IRB) approved informed consent prior to performing any procedure

### Exclusion Criteria

1. Females who are pregnant or lactating, or expecting to become pregnant during the course of the study
2. Poorly controlled diabetes mellitus as defined by having initiated intensive insulin treatment (a pump or multiple daily injections) within prior 4 months or planning to do so in the next 2 months, or 2 or more episodes of diabetic ketoacidosis requiring hospitalization within the preceding 6 months
3. Uncontrolled hypertension defined as a blood pressure >180/110 mm Hg
4. Significant co-existing disease (e.g., marked hepatic impairment, end stage renal disease (defined as a current or imminent requirement for dialysis) or symptomatic cardiac failure) that may impact on the ability of the study participant to participate in the study and/or may impact on the interpretation of the study
5. Participation in an investigational intervention clinical study within 2 months prior to study inclusion other than a non-invasive methodology or observational follow up trial in which no drugs were given
6. History of alcohol and/or drug abuse in the last 2 years
7. Men not willing to use appropriate birth control methods such as surgical sterilization or barrier contraception or men with partners of child-bearing potential not willing to use appropriate birth control methods, such as surgical sterilization, hormonal birth control (partner), an intrauterine device (partner) or double barrier method for the entire study period and for 2 months after the last dose of study drug product
8. Media clarity or pupillary dilation inadequate to obtain reasonable quality OCT and/or fundus image
9. Subjects employed by the Sponsor or in any relationship of dependence with the Sponsor and/or Investigator

### Test Product, Dose and Mode of Administration

The compound of Formula A for intravitreal injection.

Single 100 μL intravitreal injection of:
1 μg-10 μg/mL of the compound of Formula A
3 μg-30 μg/mL of the compound of Formula A
100 μg-100 μg/mL of the compound of Formula A

Criteria for evaluation

### Safety

Best Corrected Visual Acuity as measured by ETDRS EVA
Intraocular pressure
Color vision
Multifocal Electroretinogram (mfERG)
Humphrey Visual Field 24-2 (HVF 24-2)
Treatment-Emergent Adverse Events (TEAEs)
Changes on ophthalmic examination
Clinical laboratory test results
Vital signs
ECG findings

### Summary of Safety Results

Although it was not an eligibility requirement for this study, all the subjects had previously received anti-VEGF treatment in the study eye and the majority had received additional treatments including photocoagulation and intravitreal steroids.

Overall, 10 of the 14 subjects (71%) reported at least one adverse event. Severe events (1 patient) and study drug-related events (2 patients) were, however, uncommon. There were no deaths or other serious adverse events, nor did any event lead to withdrawal from the study. The majority of adverse events were consistent with those that would be expected from intravitreal injection. The most notable adverse event in the study was an episode of acute ocular pain immediately following injection secondary to a clinical diagnosis of acute increase in intraocular pressure. This diagnosis was supported by the rapid improvement in symptoms following anterior paracentesis.

There was no apparent deterioration in either mean visual acuity or mean retinal thickness. In contrast, trends of improvement in both mean visual acuity and mean retinal thickness occurred post-injection. Review of adverse events, laboratory results, ECG and physical examinations, revealed no evidence of any adverse systemic effect.

There was no evidence of any adverse effect associated with systemic exposure.

### Pharmacokinetic Results

Plasma concentrations of the compound of Formula A were quantifiable (greater than the lower limit of quantification (LLOQ), 0.25 μg/mL) in at least one sample in all subjects. Plasma concentrations of the compound of Formula A ranged from <0.25 μg/mL to 1.63 μg/mL and were quantifiable for up to 4 hours post intravitreal injection of 1 μg/eye of compound of Formula A. For the 3 μg/eye dose of the compound of Formula A, plasma concentrations of the compound of Formula A ranged from <0.25 μg/mL to 2.35 μg/mL and were quantifiable up to 24 hours post dosing. Plasma concentrations of the compound of Formula A ranged from <0.25 μg/mL to 11.3 μg/mL and were quantifiable up to 24 hours post dosing of the 10 μg/eye of compound of Formula A.

Overall, higher plasma levels of the compound of Formula A were recorded in subjects receiving the higher doses. However, it was noted that the recorded plasma levels were exceptionally low, and the ability to quantify at these levels was testament to the sensitivity of the assay. Given the known required levels for in vitro pharmacological activity of plasma kallikrein inhibition, it was considered improbable that intravitreal injection would ever result in pharmacologically meaningful systemic exposures.

### Pharmacodynamic Results

Following a single injection of the compound of Formula A as an average across all dose groups, there was a small but steady mean improvement in visual acuity at each follow up visit follow up to Day 84 (12 out of 14 subjects completed up to day 84) when all subjects were included regardless of dose. Visual acuity improved by 0.7, 1.0, 1.9, 2.8 and 4.1 letters compared with baseline at Days 7, 14, 28, 56 and 84 respectively. The mean improvement at Day 84 was greater for the 10 μg dose (5.5 letters improvement) compared with 1 μg (3.3 letters improvement) and 3 μg (2.0 letters improvement) which, although there was a small sample size, is possibly suggestive of a dose-dependent effect.

### CONCLUSION

Intravitreal injection of the compound of Formula A was well tolerated. The small number of adverse events were consistent with the route of administration rather than any observed specific drug effects. From an efficacy perspective, there was a small improvement in mean visual acuity and reduction in mean retinal thickness across the whole study population, but the small numbers and the absence of a control group preclude formal interpretation.

The results were sufficiently encouraging to warrant further investigation of the compound of Formula A in the treatment of DME in an appropriately powered, repeat-dose controlled clinical study.

Study 2—Study in Human Subjects with Center-Involving Diabetic Macular Edema (ciDME) Who have had Prior Anti-Vascular Endothelial Growth Factor (VEGF) Treatment

### Aims:

To investigate monthly dosing of intravitreal (IVT) injection of the compound of Formula A in subjects with ciDME who have had prior anti-VEGF treatment. In particular, to evaluate any effect on the efficacy in the treatment, prevention, or prevention of worsening of ciDME in subjects who have had prior anti-VEGF treatment.

To evaluate the local and systemic safety and tolerability of monthly dosing of the injections of the compound of Formula A in subjects with ciDME who had had prior anti-VEGF treatment.

### Methods:

This study was a randomized, sham-controlled, double-masked, 3-arm study into efficacy, safety and tolerability of monthly intravitreal injections of the compound of Formula A as a monotherapy in adult subjects with ciDME. The subjects had all had prior anti-VEGF treatment.

129 adult subjects were chosen using the following inclusion criteria:
1. Male or female adult subjects 18 years of age and older.
2. Confirmed diagnosis of Type I or Type II diabetes mellitus (DM). Any of the following were sufficient:
   a. Current regular use of insulin for the treatment of diabetes
   b. Current regular use of oral anti-hyperglycemia agents for the treatment of diabetes
   c. Documented diabetes by American Diabetes Association and/or World Health Organization (VVHO) criteria.
3. Best Corrected Visual Acuity (BCVA), using Standard Early Treatment Diabetic Retinopathy Study (ETDRS) chart, of ≥19 letters (˜20/400) and ≤73 letters (˜20/40) in the study eye and ≥34 letters (˜20/200 or better) in the fellow eye at Screening and Day 1.
4. Presence of ciDME in the study eye defined as Heidelberg Spectralis Optical Coherence Tomography (SD-OCT) CST ≥305 μm in women and ≥320 μm in men in the study eye (as assessed at Screening by the Investigator and Central Image Reading Center (CIRC) and on Day 1 by the Investigator).
5. Subjects' first anti-VEGF injection in the study eye occurred 36 months prior to Day 1.
6. Subjects received at least 3 anti-VEGF injections in the study eye within a 6-month period within the 36 months prior to Day 1.
7. Subject's' last anti-VEGF injection in the study eye was 8 weeks prior to Day 1.
8. Subjects, who in the view of the Investigator, were able to defer treatment in the study eye for at least 6 months following Day 1.
9. Values for blood and urine safety labs at the Screening visit showed no clinically significant deviation as determined by the Investigator.
10. Women who were post-menopausal for at least 1 year, surgically sterile for at least 3 months prior to Day 1, or who were agreeable to using highly effective contraception (adequate contraceptive measures include stable use of oral contraceptives or other prescription pharmaceutical contraceptives for two or more menstrual cycles prior to screening; intrauterine device (IUD); bilateral tubal ligation; vasectomy; condom plus contraceptive sponge, foam, or jelly or diaphragm plus contraceptive sponge, foam, or jelly), or abstinence.
11. Sexually active men who were not vasectomized and had sexual partners of childbearing potential were agreeable to using highly effective contraception.
12. Provide signed informed consent and were willing and capable of complying with clinic visits and study procedures.

Subjects were excluded from the trial if they met any of the following criteria:
1. Females who were pregnant or lactating, or expecting to become pregnant during the course of the study.
2. Evidence of ocular pathology (e.g. visually significant cataract) that impacted subject's vision in the study eye from any cause other than DME, in the opinion of the Investigator.
3. Evidence/presence of amblyopia, vitreomacular traction, epiretinal membrane, foveal atrophy, or foveal ischemia, or any other condition in the macula that was thought to impair the subject's vision (other than DME) in the opinion of Investigator.
4. Prior treatment with panretinal photocoagulation or focal grid macular photocoagulation in the study eye within the previous 3 months prior to Day 1.
5. Prior treatment with IVT steroid in the study eye (in the 3 months prior to Day 1 for triamcinolone, 6 months prior to Day 1 for Ozurdex and at any time for Iluvien).
6. Prior treatment with topical NSAIDs or topical steroids in the study eye within 1 month prior to Day 1.
7. Prior treatment with Jetrea® (ocriplasmin) injection in the study eye within the previous 3 months prior to Day 1.
8. Prior treatment with systemic corticosteroids or systemic anti-VEGF therapy within 3 months prior to Day 1.
9. Prior vitrectomy in the study eye.
10. Prior intraocular surgery in the study eye except for cataract surgery. Cataract surgery within the previous 6 months of Day 1 in the study eye was excluded.
11. Intraocular pressure (IOP) at Screening or Day 1 of >22 mmHg in the study eye or use of >2 antiglaucoma agents (combination agents count as 2 agents) in the study eye.
12. Evidence of infectious dacrocystitis, significant blepharitis, active conjunctivitis, infectious keratitis, or scleritis in either eye, or any other condition that might affect the safety of the IVT injection in the opinion of Investigator.
13. Evidence of active intraocular inflammation in the study eye.
14. Current active proliferative diabetic retinopathy (PDR), active anterior segment neovascularization (ASNV), active retinal neovascularization, or the presence of vitreous haemorrhage in the study eye. (Note, quiescent PDR is not exclusionary).
15. Any concurrent ocular condition in the study eye which, in the opinion of the Investigator, could interfere with the evaluation of efficacy or safety.
16. Poorly controlled DM defined as glycosylated hemoglobin [HgA1c]≥12.0% or having initiated intensive insulin treatment (a pump or multiple daily injections) within prior 4 months or planning to do so in the next 2 months, or two (2) or more episodes of diabetic ketoacidosis requiring hospitalization within the preceding 6 months.
17. Uncontrolled hypertension at Screening or Day 1 defined as systolic ≥180 mmHg or diastolic ≥110 mmHg.
18. Significant co-existing disease such as marked hepatic impairment, end stage renal disease (defined as a current or imminent requirement for dialysis), symptomatic cardiac failure, or significant pulmonary dysfunction that may place the subject at higher risk for treatment complications, failure of follow-up, and/or may impact the outcome of the data interpretation of the study, in the opinion of the Investigator.
19. History of other disease (e.g., unstable psychiatric illness), metabolic dysfunction, physical examination finding, or clinical laboratory finding giving reasonable suspicion of a disease or condition that contraindicated the use of an investigational product, might affect interpretation of the results of the study, or rendered the subject at high risk for treatment complications or lack of follow-up, in the opinion of Investigator.
20. History of alcohol and/or drug abuse in the last 2 years.
21. Participation in an interventional investigational clinical study within 3 months or within 5 half-lives of the last dosing of investigational drug (whichever is longer) prior to Screening.
22. Inadequate media clarity or pupillary dilation that did not allow acquisition of an adequate quality OCT and/or fundus image.

The study eye was defined as the eye that meets all of the inclusion and none of the exclusion criteria. If both eyes qualified, the eye with the worse BCVA ETDRS at Day 1 was used as the study eye. If both eyes had the same BCVA ETDRS at Day 1, the eye with the highest CST on spectral-domain optical coherence tomography (SD-OCT) on Day 1, as assessed by the Investigator, was used as the study eye. If both eyes qualified and neither was preferred based on the inclusion/exclusion criteria and had the same BCVA ETDRS and CST on Day 1, either eye was chosen as the study eye. In this instance, the Investigator selected the eye that, in their opinion, was most likely to respond to treatment as the study eye. The maximum duration of the study for each randomized subject was up to 28 weeks (including up to 4 weeks for screening, 12 weeks treatment period, and 12 weeks follow-up).

The study was conducted on an out-patient basis.

The subjects had the following baseline demographics:

TABLE 5
Baseline demographics of trial subjects

| | | | 3 μg of compound | | 6 μg of compound | | | |
|---|---|---|---|---|---|---|---|---|
| | Sham | | of Formula A | | of Formula A | | Total | |
| Variable | n = 44 | | n = 44 | | n = 41 | | n = 129 | |
| Age | | | | | | | | |
| mean (SD) | 64.6 | (8.6) | 61.1 | (10.7) | 63.2 | (9.6) | 63.0 | (9.7) |
| Range | 42-85 | | 33-83 | | 37-81 | | 33-85 | |
| Gender | | | | | | | | |
| female/male | 23/21 | (52.3/47.7) | 20/24 | (45.5/54.5) | 15/26 | (36.6/63.4) | 58/71 | (45.0/55.0) |
| n (%) | | | | | | | | |
| Race | | | | | | | | |
| white n (%) | 37 | (84.1) | 37 | (84.1) | 37 | (90.2) | 111 | (86.0) |
| black n (%) | 3 | (6.8) | 5 | (11.4) | 4 | (9.8) | 12 | (9.3) |
| other n (%) | 4 | (9.1) | 2 | (4.6) | 0 | (0) | 6 | (4.7) |
| BMI | | | | | | | | |
| mean (SD) | 31.6 | (7.0) | 33.0 | (7.7) | 31.7 | (5.4) | 32.1 | (6.8) |
| Range | 18.2-46.6 | | 22.7-58.7 | | 19.9-43.9 | | 18.2-58.7 | |
| HbA1c | | | | | | | | |
| mean (SD) | 7.5 | (1.2) | 7.6 | (1.3) | 8.0 | (1.5) | | |

| | | | |
|---|---|---|---|
| Range | 5.5-10.8 | 5.2-11.8 | 5.9-11.3 |

The study schedule of events was as follows:
1. Screening Phase:

The screening period was up to 4 weeks prior to study Day 1. All subjects signed an Informed Consent Form (ICF) prior to any study related procedures being performed. Subjects were 18 years of age or older, at the time of screening, and had a diagnosis of ciDME with prior anti-VEGF treatment.

A medical and ocular history was taken for each subject.

Each subject's DME disease history was recorded at the screening visit. The following was documented:
Date of first diagnosis of DME in the study eye
Date and details of the first anti-VEGF injection
Dates and details of the last three anti-VEGF injections
   BCVA score or Snellen equivalent of each VA assessment in the study eye beginning with the VA assessment immediately prior to the last three anti-VEGF injections
CST from each OCT assessment in the study eye beginning with the OCT assessment immediately prior to the last three anti-VEGF injections
Estimate or actual total number of anti-VEGF injections
Date of last IVT steroid injection (if any)
Estimate or actual total number of IVT steroid injections (if any)

The Investigator's assessment of the subject's response to anti-VEGF treatment after the last 3 IVT injections compared to baseline was recorded using the following scales (baseline is defined as the status of edema and vision immediately prior to the first of the 3 injections):
Edema:
   Satisfactory response—Absence of intraretinal/subretinal fluid;
   Partial response 1—Significant reduction of intraretinal/subretinal fluid;
   Partial response 2—Little or some reduction (˜20%) of intraretinal/subretinal fluid;
   No response—No reduction or worsening of intraretinal/subretinal fluid.
Vision:
   No change in or worsening of BCVA
   Gain of 1-4 letters
   Gain of 5 to 9 letters
   Gain 10 to 14 letters
   Gain of ≥15 letters

Of the study population, the patient subjects had the following distribution of times since their first anti-VEGF treatment:
<6 months-16%
Between 6 months and 1 year-29%
Between 1 and 2 years-35%
Between 2 and 3 years-20%

Previous and concomitant medication was also documented.

The following vital signs were assessed at rest (5 minutes in a supine position). The same equipment for each vital sign evaluation was used on given patient for all study visits. Vital signs were conducted prior to study drug administration and approximately 30 minutes post-study drug administration on applicable visits.
Blood pressure (SBP and DBP; mmHg);
Pulse rate (beats per minute);
Body temperature (° C.);
Respiration rate (breaths per minute).

Physical examinations were performed. The physical examination were symptom directed and include the following body systems: general appearance, skin, lymphatic, head and neck, ears, nose and throat, chest and lungs, cardiovascular, abdomen, extremities, musculoskeletal and neuromuscular.

Laboratory assessments were also conducted.

Ophthalmic findings were performed. All ophthalmic assessments were conducted on both eyes except fundus photography which was taken in both eyes at the Screening visit and only in the study eye at subsequent visits.
2. Treatment Phase:

On the day of first study drug administration (Day 1), the subject's eligibility was reconfirmed and baseline assessments were performed.

The subjects had the following baseline DME disease characteristics:

TABLE 6
Baseline DME disease characteristics of trial subjects

| | | | 3 μg of | | 6 μg of | | | |
|---|---|---|---|---|---|---|---|---|
| | | | compound of | | compound of | | | |
| | Sham | | Formula A | | Formula A | | Total | |
| Variable | n = 44 | | n = 44 | | n = 41 | | n = 129 | |
| BCVA (letters) | | | | | | | | |
| mean (SD) | 60.7 | (7.4) | 58.8 | (12.5) | 58.0 | (12.8) | 59.2 | (11.1) |
| range | 42-71 | | 23-72 | | 28-73 | | 23-73 | |
| BCVA ≤55 | | | | | | | | |
| n (%) | 11 | (25.0) | 13 | (29.5) | 11 | (26.8) | 35 | (27.1) |
| CST (μm) | | | | | | | | |
| mean (SD) | 500 | (131) | 540 | (166) | 512 | (134) | 517 | (145) |
| range | 326-815 | | 322-1,097 | | 307-804 | | 307-1,097 | |
| CST ≥450 μm | | | | | | | | |
| n (%) | 27 | (61.4) | 27 | (61.4) | 26 | (63.4) | 80 | (62.0) |
| Number of prior | | | | | | | | |
| anti-VEGF | | | | | | | | |
| injections | | | | | | | | |
| (study eye) | | | | | | | | |
| mean (SD) | 8.0 | (4.0) | 7.4 | (4.1) | 5.9 | (3.0) | 7.1 | (3.8) |
| range | 3-20 | | 3-16 | | 3-14 | | 3-20 | |
| | n = 42 | | n = 41 | | n = 38 | | n = 121 | |
| Duration of | | | | | | | | |
| DME (yrs) | | | | | | | | |
| mean (SD) | 1.4 | (1.3) | 1.0 | (0.9) | 1.0 | (1.2) | 1.1 | (1.1) |

| | | | | |
|---|---|---|---|---|
| range | 0-8 | 0-3 | 0-5 | 0-8 |

As demonstrated in Table 6 above, all subjects that took part in the study had anti-VEGF treatment prior to commencing the study. The minimum number of previous anti-VEGF injections for any patient was 3, and most patients had significantly more than 3 prior anti-VEGF injections.

The 129 eligible subjects were randomized approximately 1:1:1 into three arms:
1. Arm 1, N=44:
   Received injections of 3 μg/eye (100 μL injection volume at a concentration of 30 μg/mL) of the compound of formula A;
2. Arm 2, N=41:
   Received injections of 6 μg/eye (100 μL injection volume at a concentration of 60 μg/mL) of the compound of formula A; and
3. Arm 3, N=44:
   Received a sham procedure;
      during a 12-week, double-masked treatment period (a total of 4 doses given at approximately monthly intervals).

Subjects visited the study clinic on Day 1 and Weeks 4, 8, and 12 during the Treatment Phase for study drug administration or sham procedure, safety, and ophthalmic assessments.

The ophthalmic assessments were carried out in approximately the following order at the subjects' visits to the study clinic on Day 1, and Weeks 4, 8 and 12:
BCVA (must be performed prior to all other ophthalmic procedures)
Slit lamp biomicroscopy
Pre-Injection IOP/IOP at visits with no study drug administration (must be done prior to dilation)
Dilated indirect ophthalmoscopy
Fundus photography
SD-OCT
Intravitreal injection
Post-Injection IOP

The subject remained in the clinic after study drug administration or sham procedure until all post-dose procedures and observations were completed and the Investigator confirmed that the subject may be discharged. Investigators scheduled visits at Week 4, 8, and 12 to provide 28 days between visits. The visit window for these visits is −3 days to +7 days.

Approximately twenty-four (24) hours after each study drug administration or sham procedure on Day 1 and Weeks 4, 8, and 12, subjects were contacted by telephone to evaluate any reported AEs and changes in concomitant medications. In the event of any reported ocular or systemic AEs that were considered by the Investigator to be a possible cause for concern, the subjects returned to the clinic for assessment as soon as possible.
3. Follow up Phase:

All subjects visited the clinic at Weeks 16, 20, and 24 after the last study drug administration or sham procedure for safety and ophthalmic assessments. The visit window for Weeks 16, 20, and 24 is ±7 days.

### Early Discontinuation:

If any subject discontinued the trial early, every effort was made to complete the Week 24/early discontinuation (ED) evaluations as soon as possible and, whenever possible, prior to starting any new medication or treatment. All attempts were made to not discontinue the subject unless necessary.

### Rescue Treatment:

Rescue intervention (e.g., anti-VEGF, focal/grid macular laser photocoagulation, IVT steroids) was administered due to worsening DME (i.e., attributable to worsening DME and not another cause) if either of the following occurred and, where possible, after consultation with the Medical Monitor:
During Treatment Phase
   Best corrected visual acuity (BCVA) deteriorated 3 lines (15 letters) or more from baseline
   Central Subfield Thickness (CST) worsened >100 μm from baseline
During Follow up Phase (i.e., after week 16)
   BCVA deteriorated 3 lines (15 letters) or more from highest BCVA during treatment phase or baseline
   CST worsened >100 μm from lowest CST during treatment phase or baseline.

If a study subject met the criteria for rescue intervention and received a rescue treatment in the study eye, the study subject will be discontinued from further participation in the study.

### Assessment of Results

The assessment measured change from baseline in BCVA letter count in the study eye at Week 16. Change from baseline in BCVA letter count is calculated as Week 16 BCVA letter count minus Day 1 BCVA letter count such that a negative difference indicates a worsening in vision. In addition, treatment comparisons between each dose (Arms 1 and 2) of the injection of the compound of Formula A and the sham are calculated as the injection of the compound of Formula A minus sham.

### Study Drug

### Identity

For clinical trial use, the compound of Formula A was formulated as an injection, in accordance with the above outlined preparative methods. Any of the preparative methods outlined above in Background Examples 1 and 2 is suitable for preparing the injectable formulation of the compound of Formula A.

The injection of the compound of Formula A was supplied in two dose strengths, 60 μg/mL and 30 μg/mL free base equivalent of the compound of Formula A.

### Administration

Injection of the compound of Formula A or sham procedure was administered to the study eye on Day 1 and Weeks 4, 8, and 12 during the Treatment Phase. At each scheduled visit, the date and time of study medication administration was recorded.

The injecting physician was not the Investigator as they remained masked throughout the study. In order to avoid breaking the mask, real and sham injections were performed by study personnel who were not masked and not otherwise involved in the study (note that post-injection IOP evaluations were performed by study personnel who were unmasked). For sham injections, the subjects were prepared exactly as for a real injection (i.e., including but not limited to: insertion of lid speculum, application of povidone-iodine and subconjunctival injection of an anesthetic) following which an empty syringe with no needle was pressed against the eye to mimic the pressure of an injection.

The formulation of the compound of Formula A was supplied in three dose strengths, sham, 30 μg/mL and 60 μg/mL free base equivalent solutions. The formulation of the compound of Formula A was presented in a 2 mL Type 1 clear glass serum vial sealed with a rubber stopper and white flip off seal. Each vial was packaged in a five (5) unit container carton. The packaged kits were refrigerated (2 to 8° C.). The formulation of the compound of Formula A was administered as an intravitreal injection in a final volume of 100 μL for the 3 μg (30 μg/mL) dose, and 100 μL for the 6 μg (60 μg/mL) dose.

On the day of the first injection visit, the kit carton was pulled from the refrigerator and inspected to ensure the tamper evident seal is intact. The kit carton was not used if the tamper evident seal had been compromised. The tamper evident seal was broken, the kit carton was opened, and the vial containing the compound of Formula A, 1 of 5, was removed. The vial was warmed to room temperature for a minimum of 15 minutes. The removable panel of the vial label was peeled and affixed to the subject documentation. The investigational product vial was used for patient dosing within the same working day after removal from refrigerated storage. For subsequent dosing vials were sequentially removed from the kit and use was recorded by affixing vial specific removable panel label to subject documentation.

The flip-off seal on the vial was removed and the top was wiped with an alcohol pad. Using the provided sterile, single-use 25-gauge needle attached to a sterile, single-use 1 CC tuberculin syringe, sufficient volume (to ensure a final injectable volume of 100 μL remains in the syringe following needle exchange and removal of trapped air as described) of the formulation of the compound of Formula A was withdrawn into the syringe by inserting the needle through the rubber stopper into the vial. Once the formulation of the compound of Formula A was drawn into the syringe, the 25-gauge needle that was used to draw the drug up was replaced with a sterile, single use 30-gauge needle to perform the injection. Trapped air/bubbles and excess volume were removed to medical waste so that 100 μL of the formulation of the compound of Formula A remained in the syringe. Sterility of the needle tips was maintained as well as vial surface during preparation to ensure that there was no contamination of the drug as it was withdrawn from the vial. Unnecessary and repeated removal was avoided, as well as replacement of the needle over cap, as it decreased needle sharpness.

The dose was administered immediately upon preparation:
1. The injecting physician and a second person confirmed which eye is the study eye that received the intravitreal injection and confirmed that the patient was being dosed according to their randomization assignment. The study eye was marked with a sticker or marking pen.
2. The study eye was draped at the injecting physician's discretion, but this was not required as part of the study procedure.
3. 1-2 drops of a topical anesthetic was instilled into the study eye.
4. Povidone iodine was applied to the study eye and lashes and skin around the eye.
5. A sterile eyelid speculum was placed to stabilize the eyelids.
6. A subconjunctival anesthetic injection was administered to the study eye. This was mandatory and required for both the injection of the compound of Formula A and the sham treatment arms in order to maintain the mask in the study.
7. For the injection of the compound of Formula A arms only: the needle of syringe was inserted into the study eye 3.5-4 mm posterior to the limbus. The drug was slowly injected to gently distribute into the vitreous cavity with the needle pointing toward the optic nerve. The needle was carefully removed from the eye.
8. For sham arm only: The hub of a needleless syringe was applied to the conjunctiva and pressed gently to mimic the force of an actual injection.

Post-injection procedures—for all arms:
1. The lid speculum was removed, avoiding any excess pressure on the eye.
2. Immediately after injection, the injecting physician confirmed that the central retinal artery was perfused (even if pulsating) or checked vision to confirm that there was some perception of vision (even hand motion or light perception) in the study eye.
3. Participant's intraocular pressure (IOP) was measured within 60 minutes post IP injection by unmasked staff.
4. Subjects were contacted by telephone approximately 24 hours after each injection to evaluate AEs and changes in concomitant medications.

### Packaging, Labelling and Storage

All packaging and labelling operations were performed according to Good Manufacturing Practice for Medicinal Products and the relevant regulatory requirements.

The drug product was provided in a 2 mL Type 1 glass serum vial sealed with a rubber stopper and flip off seal. Each vial was for a single use and filled with 2 mL of the injection product of the compound of Formula A.

Supplies for control subjects took the form of boxes identical to those holding the vials of the compound of Formula A but these boxes contained empty vials. All boxes remained closed except while being accessed by the nominated unmasked personnel administering the real and sham injections.

The Investigator ensured that the drug product was stored in appropriate conditions in a secure, substantially constructed refrigerator with controlled access. Drug product was stored at 2 to 8° C. temperature except on dosing date where it could be at room temperature for up to 1 day. Upon completion of dosing, the used drug product could be destroyed with routine medical waste at the clinical site.

### Concomitant Medications/Therapy

The concomitant use of the following medications was not be allowed in the study:
Anti-VEGF administered systemically;
Any treatment in the study eye or given intravitreally in the study eye for DME other than the study medication. Note, the fellow (non-study) eye may be treated for DME at the Investigator's discretion;
Steroid administered systemically, intravitreally in the study eye, or topically in the study eye;
NSAIDs administered topically in the study eye;
Any Jetrea® (ocriplasmin) intravitreal injection in the study eye.
Any ophthalmic medication that in the opinion of the Investigator might have influenced the interpretation of the safety and/or efficacy parameters in this study.

Details of all medications (other than those intended to treat the study subjects' DME), therapies and supplements administered within 3 months prior to Screening Visit until the end of the study was recorded

With the exception of ocular medications intended to treat DME, prior medications are defined as those medications taken within 3 months prior to Screening Visit; concomitant medications are defined as those medications ongoing at or started after Day 1.

### Measurement Methods or Assessment:

The efficacy variable of interest in the study was: BCVA in letters as measured by ETDRS.

ETDRS is the Early Treatment Diabetic Retinopathy Study, measured using the Early Treatment Diabetic Retinopathy Study (ETDRS) chart

These further ophthalmic assessments (with which the skilled person would be familiar) were also used:
CST in μm as measured by Spectral Domain OCT
Retinopathy severity as measured by the DRSS and graded from fundus photography
Slit Lamp Biomicroscopy:

The eyelids, cornea, conjunctiva, anterior chamber, iris/pupil and lens were evaluated. Findings were graded as normal, abnormal non-clinically significant, or abnormal clinically significant. Slit lamp biomicroscopy was performed on both eyes and was conducted prior to study drug administration on applicable visits.
Intraocular Pressure (IOP):

IOP was assessed in both eyes at all study visits. IOP was assessed with either applanation tonometry or tonopen; the method used was consistent throughout the study. IOP was assessed at both pre- and post-injection at visits with study drug administrations. Pre-injection IOP was performed prior to dilation. Post-injection IOP was assessed within 60 minutes after study drug or sham administration and was assessed by someone who was unmasked.
Dilated Indirect Ophthalmoscopy:

The vitreous, macula, choroid, optic nerve, and retina of both eyes were assessed. Findings were graded as normal, abnormal non-clinically significant, or abnormal clinically significant. The dilated indirect ophthalmoscopy was performed on both eyes and was conducted prior to study drug administration on applicable visits.

Safety variables of interest in this study are:
AEs;
Ophthalmic and physical findings;
Laboratory test results (clinical chemistry, hematology, and urinalysis);
Vital signs (SBP, DBP, PR, and respiratory rate).

### Results:

TABLE 7
Efficacy assessment - measurement of BCVA at week 16

| | | | 3 μg of | | 6 μg of | |
|---|---|---|---|---|---|---|
| | | | compound of | | compound of | |
| | Sham | | Formula A | | Formula A | |
| BCVA (letters) | n = 44 | | n = 44 | | n = 41 | |
| Any loss from baseline | | | | | | |
| n (%) | 24 | (54.5) | 22 | (50.0) | 13 | (32.5) |
| p-value (trend) | | | | | 0.0496 | |
| Diff. v. sham | | | −4.5 | (0.6695) | −22.0 | (0.0421) |
| (p-value) | | | | | | |
| ≥5 letter loss from baseline | | | | | | |
| n (%) | 12 | (27.3) | 11 | (25.0) | 8 | (20.0) |
| p-value (trend) | | | | | 0.4503 | |
| Diff. v. sham (p-value) | | | −2.3 | (0.8083) | −7.3 | (0.4344) |
| ≥10 letter loss from baseline | | | | | | |
| n (%) | 9 | (20.5) | 4 | (9.1) | 2 | (5.0) |
| p-value (trend) | | | | | 0.0280 | |
| Diff. v. sham (p-value) | | | −11.4 | (0.228) | −15.5 | (0.0516) |
| ≥15 letter loss from baseline | | | | | | |
| n (%) | 5 | (11.4) | 2 | (4.5) | 0 | (0) |
| p-value (trend) | | | | | 0.0279 | |
| Diff. v. sham (p-value) | | | −6.8 | (0.4336) | −11.4 | (0.0565) |

TABLE 8
Change in BCVA letters versus sham over time for
3 μg of compound of Formula A and 6 μg of
compound of Formula A (positive value is improvement)

| BCVA improvement | 3 μg of compound | 6 μg of compound |
|---|---|---|
| (letters) | of Formula A | of Formula A |
| 4 weeks - mean | 0.6 | 0.9 |
| 8 weeks - mean | 1.2 | 1.1 |
| 12 weeks - mean | 3.8 | 2.9 |
| 16 weeks - mean | 1.5 | 2.6 |
| 20 weeks - mean | −1.2 | 2.0 |
| 24 weeks - mean | −0.7 | 2.1 |

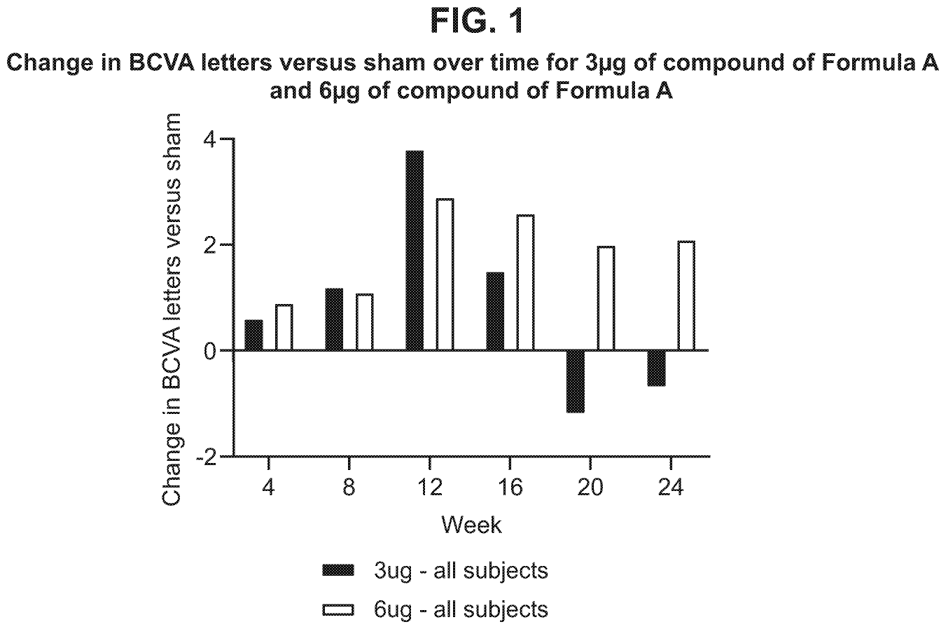

These results are also shown graphically in FIG. 1.

TABLE 9
Change in BCVA letters versus sham over time
for early stage compared with all subjects
on the dose of 6 μg of compound of Formula A

| | 6 μg of compound of | |
|---|---|---|
| | Formula A - early stage | |
| BCVA improvement | subjects (baseline | 6 μg of compound of |
| (letters) | BCVA >55 letters) | Formula A - all subjects |
| 4 weeks - mean | 2.3 | 0.9 |
| 8 weeks - mean | 1.8 | 1.1 |
| 12 weeks - mean | 4.7 | 2.9 |
| 16 weeks - mean | 4.9 | 2.6 |
| 20 weeks - mean | 3.5 | 2.0 |
| 24 weeks - mean | 2.9 | 2.1 |

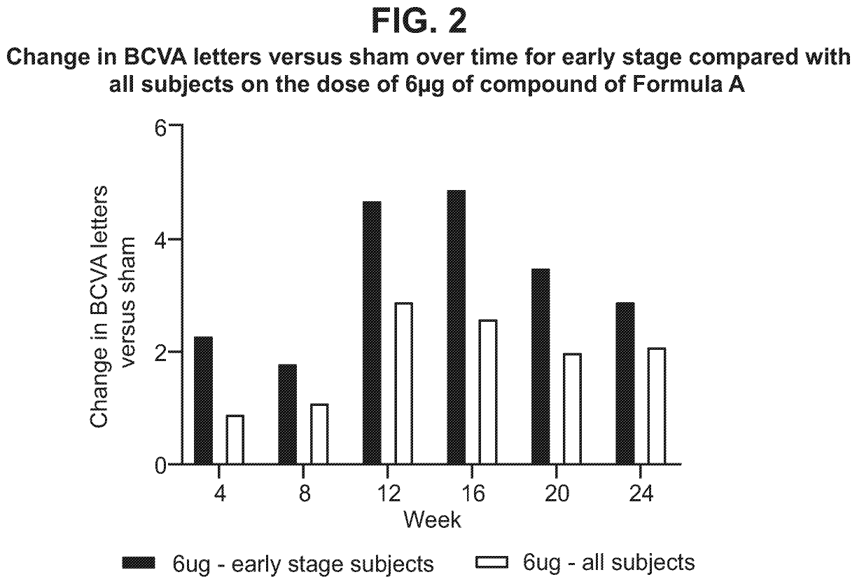

These results are also shown graphically in FIG. 2.

The majority of any reported adverse events (AEs) were mild. Two AEs led to discontinuation, one retinal neovascularization (6 μg arm) and one visual impairment (sham arm). All AEs except for the retinal neovascularization (6 μg arm) were considered unrelated to the treatment. Therefore, in >99% of the subjects, the treatment was safe and well-tolerated.

As demonstrated in Table 7, administration of the compound of Formula A to subjects that had previously had anti-VEGF treatment resulted in a slowing of the progression of their DME or impaired visual acuity. 22 patients in the group that were administered doses of 3 μg of the compound of Formula A showed any loss in BCVA (letters) from baseline compared to 24 patients that had the sham procedure. Even more markedly, only 13 patients in the group that were administered doses of 6 μg of the compound of Formula A showed any loss in BCVA (letters) from baseline compared to 24 patients that had the sham procedure. Further, 5 patients that had the sham procedure lost ≥15 letters from baseline, whereas only 2 patients in the 3 μg of the compound of Formula A group, and 0 patients in the 6 μg of the compound of Formula A group lost ≥15 letters from baseline.

The results in Table 8 demonstrate that, for the patients that were administered doses of 6 μg of the compound of Formula A that the improvement in BCVA (letters) score, compared to the sham treatment, was maintained following the 16 week treatment period, through to the 24 week time period. It appears that this effect would be seen for doses greater than those for the group of patients that were administered the 3 μg doses of the compound of Formula A. These data point towards the efficacy of a higher dose treatment, i.e. the 6 μg of the compound of Formula A, as well as higher doses.

The results in Table 9 demonstrate that the population of patients that had a baseline BCVA score of greater than 55 letters (i.e. ≥56 letters), who may be referred to as the patients in the early stages of their DME or poor visual acuity, on average, and at every measurement taken (both during the treatment procedure and in the follow up phase) had a consistently better average improvement in BCVA score than the overall average population score. Therefore, the treatment represents an efficacious treatment particularly for those patients that were in the early stages of DME or poor visual acuity.

It will be understood that the invention has been described by way of example only and modifications may be made whilst remaining within the scope and spirit of the invention.

1. The compound of Formula A (or a pharmaceutically acceptable salt and/or solvate thereof) for use in treating diabetic macular edema (DME) or impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof, 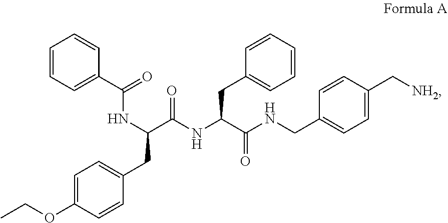 [SMILES: CCOc1ccc(C[C@@H](NC(=O)c2ccccc2)C(=O)N[C@@H](Cc2ccccc2)C(=O)NCc2ccc(CN)cc2)cc1]
wherein the patient has previously had anti-VEGF (vascular endothelial growth factor) treatment.

2. The compound of Formula A (or a pharmaceutically acceptable salt and/or solvate thereof) for use according to claim 1,
wherein the pharmaceutical composition is an aqueous solution comprising the compound of Formula A (or a pharmaceutically acceptable salt and/or solvate thereof).

3. The compound of Formula A (or a pharmaceutically acceptable salt and/or solvate thereof) for use according to claim 1,
wherein intravitreal administration comprises intravitreal injection.

4. The compound of Formula A (or a pharmaceutically acceptable salt and/or solvate thereof) for use according to claim 1,
wherein the pharmaceutical composition is intravitreally administered into at least one of the patient's eye; optionally wherein the pharmaceutical composition is intravitreally administered into both of the patient's eyes.

5. The compound of Formula A (or a pharmaceutically acceptable salt and/or solvate thereof) for use according to claim 1,
wherein the solution further comprises at least one non-ionic tonicity agent and/or histidine; preferably wherein the at least one non-ionic tonicity agent is trehalose;
preferably wherein the trehalose is provided as trehalose dihydrate.

6. (canceled)

7. The compound of Formula A (or a pharmaceutically acceptable salt and/or solvate thereof) for use according to claim 1,
wherein the pharmaceutical composition comprises an aqueous solution of the compound of formula A (or a pharmaceutically acceptable salt and/or solvate thereof), histidine and trehalose dihydrate.

8. The compound of Formula A (or a pharmaceutically acceptable salt and/or solvate thereof) for use according to claim 1,
wherein the pharmaceutical composition has a pH from about 2 to about 10, preferably from about 5 to about 7.5, preferably from about 5.3 to about 6, and preferably from about 5.4 to about 5.8; preferably wherein the pharmaceutical composition has a pH of about 5.5.

9. The compound of Formula A (or a pharmaceutically acceptable salt and/or solvate thereof) for use according to claim 1,
wherein the concentration of the compound of Formula A (or a pharmaceutically acceptable salt and/or solvate thereof) when administered is between about 10 μg/mL and about 200 μg/mL based on the concentration of the free base of the compound of Formula A in solution;
optionally wherein the concentration of the compound of Formula A (or a pharmaceutically acceptable salt and/or solvate thereof) when administered is between about 20 μg/mL and about 200 μg/mL based on the concentration of the free base of the compound of Formula A in solution;
optionally wherein the concentration of the compound of Formula A (or a pharmaceutically acceptable salt and/or solvate thereof) when administered is between about 20 μg/mL and about 160 μg/mL based on the concentration of the free base of the compound of Formula A in solution;
optionally wherein the concentration of the compound of Formula A (or a pharmaceutically acceptable salt and/or solvate thereof) when administered is between about 20 μg/mL and about 120 μg/mL based on the concentration of the free base of the compound of Formula A in solution;
optionally wherein the concentration of the compound of Formula A (or a pharmaceutically acceptable salt and/or solvate thereof) when administered is between about 20 μg/mL and about 100 μg/mL based on the concentration of the free base of the compound of Formula A in solution.

10. The compound of Formula A (or a pharmaceutically acceptable salt and/or solvate thereof) for use according to claim 1,
wherein the concentration of the compound of Formula A (or a pharmaceutically acceptable salt and/or solvate thereof) when administered is between about 30 μg/mL and about 100 μg/mL based on the concentration of the free base of the compound of Formula A in solution;
optionally wherein the concentration of the compound of Formula A (or a pharmaceutically acceptable salt and/or solvate thereof) when administered is between about 60 μg/mL and about 100 μg/mL based on the concentration of the free base of the compound of Formula A in solution; or
wherein the concentration of the compound of Formula A (or a pharmaceutically acceptable salt and/or solvate thereof) when administered is about 30 μg/mL based on the concentration of the free base of the compound of Formula A in solution; or,
wherein the concentration of the compound of Formula A (or a pharmaceutically acceptable salt and/or solvate thereof) when administered is about 60 μg/mL based on the concentration of the free base of the compound of Formula A in solution; or,
wherein the concentration of the compound of Formula A (or a pharmaceutically acceptable salt and/or solvate thereof) when administered is about 100 μg/mL based on the concentration of the free base of the compound of Formula A in solution; or
wherein the concentration of the compound of Formula A (or a pharmaceutically acceptable salt and/or solvate thereof) when administered is about 120 μg/mL based on the concentration of the free base of the compound of Formula A in solution; or,
wherein the concentration of the compound of Formula A (or a pharmaceutically acceptable salt and/or solvate thereof) when administered is about 200 μg/mL based on the concentration of the free base of the compound of Formula A in solution.

11-12. (canceled)

13. The compound of Formula A (or a pharmaceutically acceptable salt and/or solvate thereof) for use according to claim 1,
wherein about 10 μL to about 100 μL of the solution is administered per intravitreal administration;
optionally wherein about 50 μL to about 100 μL of the solution is administered per intravitreal administration; or
wherein about 100 μL of the solution is administered per intravitreal administration; or
wherein about 50 μL of the solution is administered per intravitreal administration.

14. (canceled)

15. The compound of Formula A (or a pharmaceutically acceptable salt and/or solvate thereof) for use according to claim 1,
wherein prior to administration of the compound of Formula A, the patient has a baseline visual acuity score (BCVA) of between 19 and 73 letters in at least one eye, measured using the standard Early Treatment Diabetic Retinopathy Study (ETDRS) chart.

16. The compound of Formula A (or a pharmaceutically acceptable salt and/or solvate thereof) for use according to claim 1,
wherein the patient is in the early stages of DME;
optionally wherein a patient in the early stages of DME is defined by having a baseline visual acuity score (BCVA), prior to administration of the compound of Formula A, of between 56 and 73 letters in at least one eye, measured using the standard Early Treatment Diabetic Retinopathy Study (ETDRS) chart.

17. The compound of Formula A (or a pharmaceutically acceptable salt and/or solvate thereof) for use according to claim 1,
wherein the treatment is a monotherapy for DME.

18. The compound of Formula A (or a pharmaceutically acceptable salt and/or solvate thereof) for use according to claim 1,
wherein the anti-VEGF treatment is selected from aflibercept (Eylea®), bevacizumab, ranibizumab, and pegaptanib;
preferably wherein the anti-VEGF is aflibercept (Eylea®).

19. The compound of Formula A (or a pharmaceutically acceptable salt and/or solvate thereof) for use according to claim 1,
wherein the patient received anti-VEGF treatment for no more than 36 months before commencing treatment with the compound of Formula A (or a pharmaceutically acceptable salt and/or solvate thereof); and/or
wherein the patient received anti-VEGF treatment no less than 8 weeks before commencing treatment with the compound of formula A (or a pharmaceutically acceptable salt and/or solvate thereof); and/or
wherein the patient does not receive anti-VEGF treatment concurrent to administration of the compound of Formula A.

20. (canceled)

21. The compound of Formula A (or a pharmaceutically acceptable salt and/or solvate thereof) for use according to claim 1,
wherein the treatment is administered over a time period of at least about 12 weeks; or
wherein the treatment is administered between about once every 4 weeks and about once every 12 weeks; or
wherein the treatment is administered about once every 4 weeks.

22. The compound of Formula A (or a pharmaceutically acceptable salt and/or solvate thereof) for use according to claim 1,
wherein the treatment is administered at a first dosing frequency over a first time period, followed by a second dosing frequency over a second time period, wherein the second dosing frequency is lower than the first dosing frequency.

23. The compound of Formula A (or a pharmaceutically acceptable salt and/or solvate thereof) for use according to claim 22,
wherein the first time period is greater than about 8 weeks;
optionally wherein the first time period is greater than about 12 weeks; and/or
wherein the first dosing frequency is between about once every three weeks and about once every five weeks.

24. (canceled)

25. The compound of Formula A (or a pharmaceutically acceptable salt and/or solvate thereof) for use according to claim 22,
wherein the second time period is greater than about 8 weeks; or,
wherein the second time period is between about 8 weeks and about 12 weeks; or,
wherein the second time period is about 12 weeks; and/or
wherein the second dosing frequency is lower than about once every six weeks.

26-28. (canceled)

29. The compound of Formula A (or a pharmaceutically acceptable salt and/or solvate thereof) for use in treating diabetic macular edema (DME) or impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof, 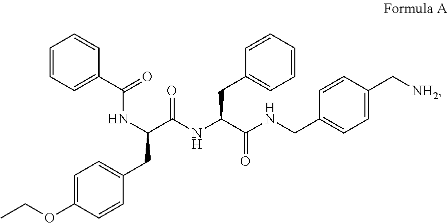 [SMILES: CCOc1ccc(C[C@@H](NC(=O)c2ccccc2)C(=O)N[C@@H](Cc2ccccc2)C(=O)NCc2ccc(CN)cc2)cc1]
wherein the patient is in the early stages of DME or impaired visual acuity.

30. (canceled)

31. The compound of Formula A (or a pharmaceutically acceptable salt and/or solvate thereof) for use according to claim 29,
wherein a patient in the early stages of DME or impaired visual acuity is defined by having a baseline visual acuity score (BCVA), prior to administration of the compound of Formula A, of between 56 and 73 letters in at least one eye, measured using the standard Early Treatment Diabetic Retinopathy Study (ETDRS) chart.

32. The compound of Formula A (or a pharmaceutically acceptable salt and/or solvate thereof) for use according to claim 29,
wherein the concentration of the compound of formula A (or a pharmaceutically acceptable salt and/or solvate thereof) when administered is between about 30 μg/mL and 100 μg/mL based on the concentration of the free base of the compound of formula A in solution; or,
wherein the concentration of the compound of formula A (or a pharmaceutically acceptable salt and/or solvate thereof) when administered is between about 60 μg/mL and 100 μg/mL based on the concentration of the free base of the compound of formula A in solution; or
wherein the concentration of the compound of formula A (or a pharmaceutically acceptable salt and/or solvate thereof) when administered is about 60 μg/mL based on the concentration of the free base of the compound of formula A in solution.

33. (canceled)

34. The compound of Formula A (or a pharmaceutically acceptable salt and/or solvate thereof) for use in treating diabetic macular edema (DME) or impaired visual acuity comprising: intravitreally administering a pharmaceutical composition, wherein the pharmaceutical composition is a solution comprising the compound of Formula A (or a pharmaceutically acceptable salt and/or solvate thereof), to a patient in need thereof, 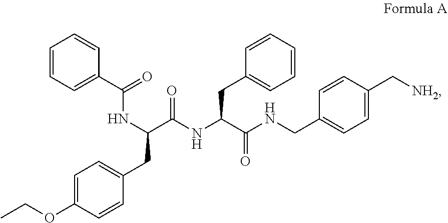 [SMILES: CCOc1ccc(C[C@@H](NC(=O)c2ccccc2)C(=O)N[C@@H](Cc2ccccc2)C(=O)NCc2ccc(CN)cc2)cc1]
wherein the treatment is administered at a first dosing frequency over a first time period wherein the concentration of the compound of formula A (or a pharmaceutically acceptable salt and/or solvate thereof) when administered is greater than about 30 μg/mL based on the concentration of the free base of the compound of formula A in solution, followed by a second dosing frequency over a second time period, wherein the second dosing frequency is lower than the first dosing frequency.

35. (canceled)

36. The compound of Formula A (or a pharmaceutically acceptable salt and/or solvate thereof) for use according to claim 34,
wherein the treatment is administered at a first dosing frequency over a first time period;
wherein the concentration of the compound of formula A (or a pharmaceutically acceptable salt and/or solvate thereof) when administered is between about 60 μg/mL and about 100 μg/mL based on the concentration of the free base of the compound of formula A in solution.

37. The compound of Formula A (or a pharmaceutically acceptable salt and/or solvate thereof) for use according to claim 34,
wherein the first time period is greater than about 8 weeks; or,
wherein the first time period is greater than about 12 weeks; and/or
wherein the second time period is greater than about 8 weeks; or
wherein the second time period is between about 8 weeks and about 12 weeks; or,
wherein the second time period is about 12 weeks.

38. The compound of Formula A (or a pharmaceutically acceptable salt and/or solvate thereof) for use according to claim 34,
wherein the first dosing frequency is between about once every three weeks and about once every five weeks; and/or
wherein the second dosing frequency is lower than about once every six weeks.

39-40. (canceled)

41. The compound of Formula A (or a pharmaceutically acceptable salt and/or solvate thereof) for use according to claim 1,
wherein the previous anti-VEGF treatment was for treating impaired visual acuity or DME.

42. The compound of Formula A (or a pharmaceutically acceptable salt and/or solvate thereof) for use according to claim 1,
wherein the treatment with the solution comprising the compound of Formula A (or a pharmaceutically acceptable salt and/or solvate thereof) slows the progression of impaired visual acuity or DME.
